(19) United States
(12) Patent Application Publication
(10) Pub. No.: US 2025/0041292 A1
(43) Pub. Date: Feb. 6, 2025

(54) METHODS FOR TREATING PRIMARY IMMUNODEFICIENCY

(71) Applicant: X4 Pharmaceuticals, Inc., Boston, MA (US)

(72) Inventors: Art Taveras, Boston, MA (US); Katarina Zmajkovicova, Bratislava (SK); Chi Nguyen, Vienna (AT)

(21) Appl. No.: 18/717,360

(22) PCT Filed: Dec. 9, 2022
(86) PCT No.: PCT/US2022/052386

§ 371 Date: Jun. 6, 2024

Related U.S. Application Data
(60) Provisional application No. 63/265,259, filed on Dec. 10, 2021.
(60) Provisional application No. 63/265,261, filed on Dec. 10, 2021.
(60) Provisional application No. 63/265,263, filed on Dec. 10, 2021.
(60) Provisional application No. 63/265,264, filed on Dec. 10, 2021.
(60) Provisional application No. 63/265,265, filed on Dec. 10, 2021.
(60) Provisional application No. 63/265,185, filed on Dec. 9, 2021.

(51) Int. Cl. A61K 31/496; A61K 31/444; A61K 31/4709; A61P 37/06

(52) U.S. Cl. CPC A61K 31/496; A61K 31/444; A61K 31/4709; A61P 37/06

(57) ABSTRACT

The present invention relates to methods of treating patients with primary immunodeficiency diseases and disorders, using a CXCR4 inhibitor.

### CROSS-REFERENCE TO RELATED APPLICATIONS

This application claims the benefit of U.S. Provisional Application Nos. 63/265,185, filed Dec. 9, 2021; 63/265,259, filed Dec. 10, 2021; 63/265,261, filed Dec. 10, 2021; 63/265,263, filed Dec. 10, 2021; 63/265,264, filed Dec. 10, 2021; and 63/265,265, filed Dec. 10, 2021; the entirety of each of which is hereby incorporated by reference.

### FIELD OF THE INVENTION

The present invention relates to methods for treating primary immunodeficiency diseases and disorders using a compound that inhibits CXC Receptor type 4 (CXCR4).

### BACKGROUND OF THE INVENTION

Peripheral leukocyte deficiency is a common feature of multiple diseases and may render affected individuals susceptible to infections, both common and opportunistic. The CXCR4 chemokine receptor regulates the trafficking of leukocytes among the bone marrow, blood, and lymphatic system (Al Ustwani O, et al. Br J Haematol. 2014; 164:15-23). It is known to be involved with some immunodeficiencies such as WHIM syndrome, but not all human primary immunodeficiency diseases (PIDs) involve a mutation or alteration in CXCR4 function. PIDs comprise 330 distinct disorders with 320 different gene defects listed. Long considered as rare diseases, recent studies tend to show that they are more common than generally thought, if only by their rapidly increasing number. The International Union of Immunological Societies (IUIS) PID expert committee proposed a PID classification since 1999, which facilitates clinical research and comparative studies worldwide; it is updated every other year to include new disorders or disease-causing genes.

For example, neutropenia is a condition characterized by an abnormally low concentration of neutrophils circulating in the blood, and defined by an absolute neutrophil count (ANC) below 1500 cells/μL. Severe neutropenia (ANC<500 cells/μL) is a risk factor for susceptibility to bacterial infection. Neutrophils make up the majority of circulating white blood cells and play an important role in the body's defenses against bacterial or fungal pathogenic infections and in shaping the host response to infection. In addition, neutrophils participate in immune system homeostasis. Neutropenia can be divided into congenital (i.e., present at birth) and acquired. Additionally, neutropenia can be “acute” (transient, or temporary, often as a response to specific events that deplete the body of neutrophils, such as radiation or chemotherapy), or “chronic” (a long-term or long-lasting effect that may be due to the presence of genetic abnormalities).

Acute or transient neutropenia can be caused by infectious agents, such as the typhoid-causing bacterium Salmonella enterica; and cytomegalovirus, as well as chemical agents, including propylthiouracil; levamisole; penicillamine; clozapine; valproic acid; and cancer chemotherapy. Chronic neutropenia can be caused by genetic abnormalities (congenital neutropenia). Mutations in ELANE are the most common cause of congenital neutropenia. Other examples of genes that can be responsible for genetic causes of neutropenia include HAX1, G6PC3, WAS, SBDS, and others. In addition, some enzyme deficiencies can be associated with neutropenia such as glycogen storage disease lb. Other causes of neutropenia include mitochondrial diseases, such as Pearson syndrome. Some autoimmune diseases, such as systemic lupus erythematosus (“SLE” or “lupus”) may be associated with neutropenia. Aplastic anemia, due to bone marrow failure, is associated with thrombocytopenia, anemia and neutropenia; Evans syndrome is characterized by autoimmune hemolytic anemia (AIHA) and immune thrombocytopenia (ITP) and/or immune neutropenia; and Felty's syndrome is characterized by rheumatoid arthritis, splenomegaly and neutropenia. Chronic neutropenia may also be the result of nutritional deficiencies, such as abnormally low levels of copper or Vitamin B12; or chronic infections, such as with human immunodeficiency virus (HIV), the agent that causes AIDS.

Neutropenia may be asymptomatic and often is only diagnosed fortuitously. Today, the standard treatment for severe neutropenia is administration of granulocyte colony-stimulating factor (G-CSF). Historically, neutropenia has been treated in a host of manners, including splenectomy, corticosteroids, androgens, and immunosuppressive and immune-modulating therapies. Currently, however, these treatments are generally not recommended except in cases where treatment with G-CSF is not effective. Dale et al. (2017) Curr. Opin. Hematol. 24:46-53; Sicre de Fontbrune et al. (2015) Blood 126:1643-1650. Other treatments for neutropenia can include bone marrow transportation and/or treatment with cord blood stem cells.

There exists a need for improved treatments capable of reversing peripheral leukocyte deficiency, neutropenia, and other immune cell deficiencies. The present invention addresses this need and provides other related advantages.

### SUMMARY OF THE INVENTION

In one aspect, the present invention provides a method for treating a primary immunodeficiency (PID) in a subject in need thereof, comprising administering to the subject an effective amount of a CXCR4 inhibitor or a pharmaceutically acceptable salt or composition thereof.

In some embodiments, the CXCR4 inhibitor is selected from mavorixafor,

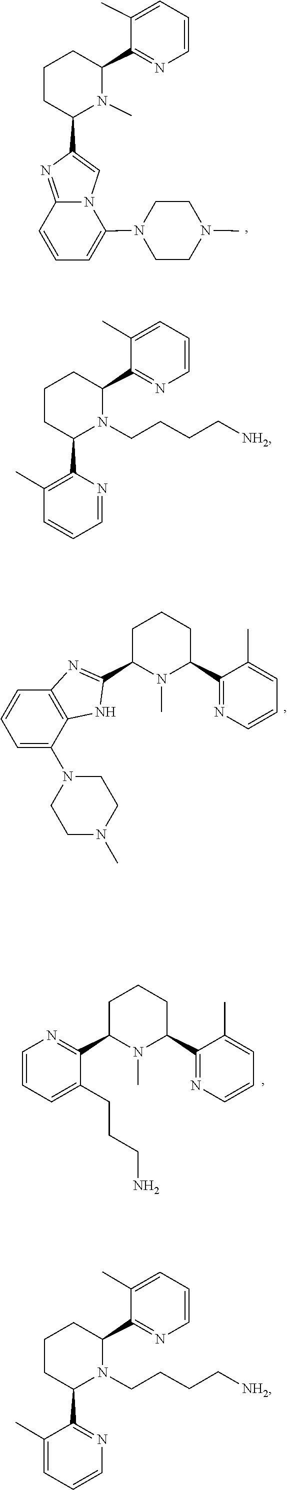

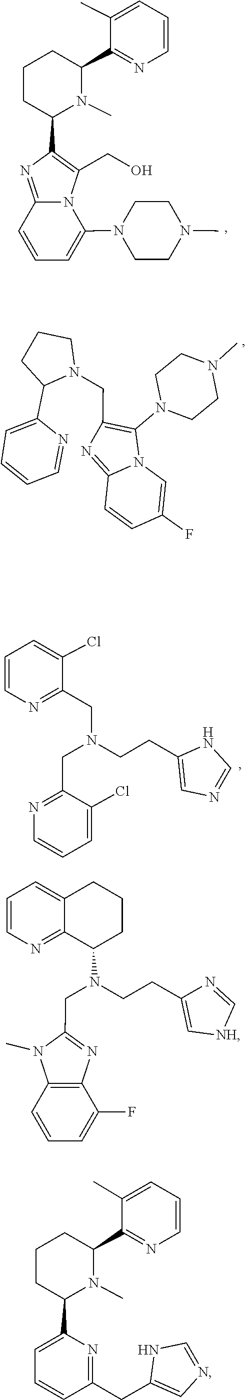

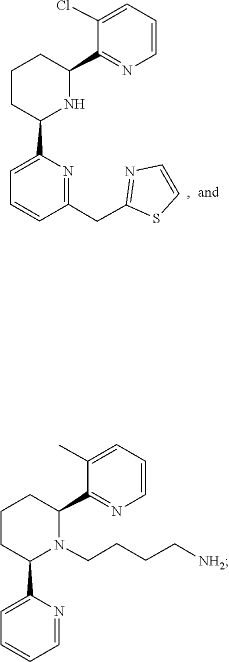 [SMILES: Clc1cccnc1[C@@H]1CCC[C@H](c2cccc(Cc3nccs3)n2)N1 ; Cc1cccnc1[C@@H]1CCC[C@H](c2ccccn2)N1CCCCN]

or a pharmaceutically acceptable salt or composition thereof.

In some embodiments, the CXCR4 inhibitor is plerixafor or a pharmaceutically acceptable salt thereof.

In some embodiments, the CXCR4 inhibitor is mavorixafor or a pharmaceutically acceptable salt or composition thereof.

In some embodiments, the CXCR4 inhibitor is:

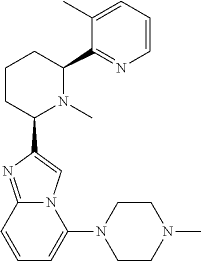 [SMILES: Cc1cccnc1[C@@H]1CCC[C@H](c2cn3c(N4CCN(C)CC4)cccc3n2)N1C]

or a pharmaceutically acceptable salt thereof.

In some embodiments, the CXCR4 inhibitor is selected from the following:

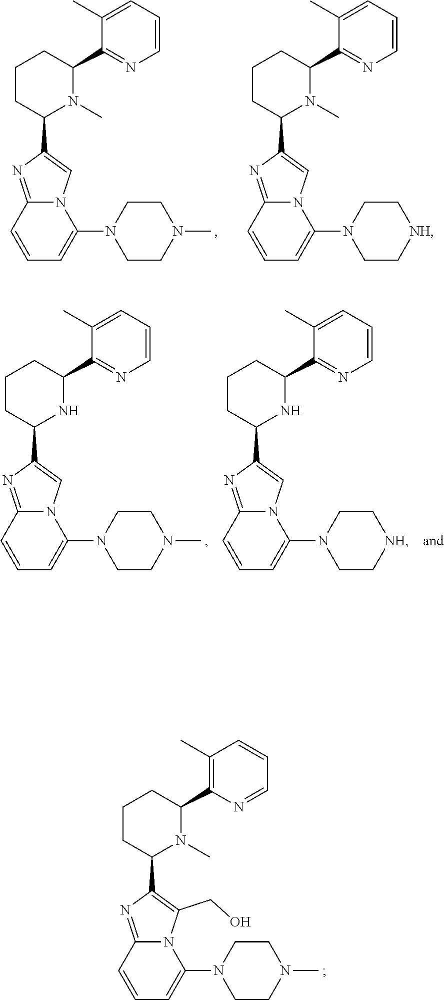

or a pharmaceutically acceptable salt thereof.

In some embodiments, the PID is a disease or disorder associated with innate immunity defects.

In some embodiments, the disease or disorder associated with innate immunity defects is selected from predisposition to invasive bacterial infections comprising meningitis, sepsis, osteomyelitis, and/or abscesses; IRAK4 deficiency comprising skin infections and upper respiratory tract infections; IRAK-1 deficiency comprising X-linked MECP2 deficiency-related syndrome; TIRAP, comprising staphylococcal disease during childhood; and isolated congenital asplenia, comprising bacteremia, no-spleen, hemolysis, nephritis, and inflammation.

In some embodiments, the disease or disorder associated with innate immunity defects is TRAK4 deficiency and wherein the patient has a mutation in IRAK4 gene; wherein the disease or disorder associated with innate immunity defects is IRAK-1 deficiency and wherein the patient has a mutation in IRAK1 gene; wherein the disease or disorder associated with innate immunity defects is TIRAP deficiency and wherein the patient has a mutation in TIRAP gene; wherein the disease or disorder associated with innate immunity defects is isolated congenital asplenia and wherein the patient has a mutation in RPSA gene; or wherein the disease or disorder associated with innate immunity defects is isolated congenital asplenia and wherein the patient has a mutation in HMOX gene.

In some embodiments, the disease or disorder associated with innate immunity defects is selected from predisposition to parasitic and fungal infections; mucocutaneous candidiasis, comprising chronic mucocutaneous candidiasis without ectodermal dysplasia; treating STAT1 (GOF), comprising fungal infections, bacterial infections, viral infections, HSV, autoimmunity, thyroiditis, diabetes, cytopenias, and enteropathy; IL-17F deficiency, comprising folliculitis; IL-17RA deficiency, comprising folliculitis, susceptibility to S. aureus and susceptibility to skin infections; IL-17RC deficiency; ACT1 deficiency, comprising blepharitis, folliculitis and macroglossia; CARD9 deficiency, comprising predisposition to invasive fungal diseases, predisposition to invasive candidiasis infection, and deep dermatophytosis; and trypanosomiasis.

In some embodiments, the disease or disorder associated with innate immunity defects is STAT1 (GOF) and wherein the patient has a mutation in STAT1 gene; the disease or disorder associated with innate immunity defects is IL-17F deficiency and wherein the patient has a mutation in IL17F gene; the disease or disorder associated with innate immunity defects is IL-17RA deficiency and wherein the patient has a mutation in IL17RA gene; the disease or disorder associated with innate immunity defects is IL-17RC deficiency and wherein the patient has a mutation in IL17RC gene; the disease or disorder associated with innate immunity defects is ACT1 deficiency and wherein the patient has a mutation in ACT1 gene; the disease or disorder associated with innate immunity defects is CARD9 deficiency and wherein the patient has a mutation in CARD9 gene; or wherein the disease or disorder associated with innate immunity defects is trypanosomiasis and wherein the patient has a mutation in APOL1 gene.

In some embodiments, the disease or disorder associated with innate immunity defects is selected from osteopetrosis, comprising hypocalcemia, neurologic features and severe growth retardation; hidradenitis suppurativa, comprising acne and hyperpigmentation; acute liver failure due to NBAS deficiency, comprising fever induced liver failure; acute necrotizing encephalopathy, comprising fever induced acute encephalopathy; and IRF4 haploinsufficiency, comprising Whipple's disease.

In some embodiments, the disease or disorder associated with innate immunity defects is osteopetrosis and wherein the patient has mutations in one or more genes selected from TNFRSF11A, PLEKHM1, and TCIRG1; wherein the disease or disorder associated with innate immunity defects is hidradenitis suppurativa and wherein the patient has mutations in one or both of PSENEN and NCSTN genes; or wherein the disease or disorder associated with innate immunity defects is acute liver failure due to NBAS deficiency and wherein the patient has a mutation in NBAS gene; wherein the disease or disorder associated with innate immunity defects is acute necrotizing encephalopathy and wherein the patient has a mutation in RANBP2 gene; or wherein the disease or disorder associated with innate immunity defects is IRF4 haploinsufficiency and wherein the patient has a mutation in IRF4 gene.

In some embodiments, the disease or disorder associated with innate immunity defects is selected from severe phenotypes of Mendelian susceptibility to mycobacterial disease, complete IFNGR1 deficiency, moderate phenotypes of Mendelian susceptibility to mycobacterial disease, IL-12 and IL-23 receptor b1 chain deficiency, IL-12Rb2 deficiency, IL-23R deficiency, STAT1 (LOF), partial IFNγR1, partial IFNγR2, AD IFNGR1, partial SPPL2a deficiency, partial Tyk2 deficiency, macrophage gp91 phox deficiency, IRF8 deficiency, IFG15 deficiency, RORγT deficiency, JAK1 (LOF), epidermodysplasia verruciformis (HPV), partial EVER1 deficiency, partial EVER2 deficiency, partial CIB1 deficiency, WHIM, predisposition to severe viral infection, STAT1 deficiency, STAT2 deficiency, IRF7 deficiency, IRF9 deficiency, IFNAR1 deficiency, IFNAR2 deficiency, CD16 deficiency, MDAS deficiency, RNA polymerase III deficiency, IL-18BP deficiency and herpes simplex encephalitis.

In some embodiments, the patient has mutations in one or more genes selected from IFNGR1, IFNGR2, IL12RB1, IL12RB2, IL23R, STAT1, IFNGR1, IFNGR2, SPPL2A, TYK2, CYBB, IRF8X, ISGJS, RORC, JAK1, TMC6, TMC8, CIB1, CXCR4, STAT2, IRF7, IRF9, IFNAR1, IFNAR2, FCGR3A, IFIH1, POLR3A, POLR3C, POLR3F, IL18BP, UNC93B1, TRAF3, TICAM1, TBK1 and IRF3.

In some embodiments, the PID is a disease or disorder associated with functional defects of phagocytes.

In some embodiments, the disease or disorder associated with functional defects of phagocytes is selected from Leukocyte Adhesion Deficiency Type 1, Leukocyte Adhesion Deficiency Type 2, Leukocyte Adhesion Deficiency Type 3, pulmonary alveolar proteinosis, chronic granulomatous disease, Rac-2 deficiency and G6PD deficiency Class 1.

In some embodiments, the patient has mutations in one or more genes selected from, ITGB2, SLC35C1, FERMT3, CSF2RA, CSF2RB, NCF1, CYBA, NCF4, NCF2, CYBC1, RAC2 and G6PD.

In some embodiments, the disease or disorder associated with primary immunodeficiency is selected from DiGeorge Syndrome, CHARGE syndrome, Chromosome 10p13-p14 deletion syndrome, Jacobsen syndrome, FOXN1 haploinsufficiency, cartilage hair hypoplasia, Schimke Syndrome, MOPD1 deficiency, MYSM1 deficiency, Facial dysmorphism, Immunodeficiency, Livedo, and Short stature (FILS) Syndrome and Mannose-Binding Lectin (MBL) deficiency.

In some embodiments, the patient has mutations in one or more genes selected from, TBX1, CHD7, SEMA3E, 10p13-p14DS, 11q23DEL, FOXN, RMRP, SMARCAL1, RNU4ATAC, MYSM1, POLE, MASP2 and FCN3.

In some embodiments, the PID is a common variable immune deficiency (CVID) or a disease or disorder associated with CVID.

In some embodiments, the CVID or disease or disorder associated with CVID is selected from recurrent infections, polyclonal lymphoproliferation, autoimmune cytopenias, granulomatous disease, severe bacterial infections, PTEN deficiency, activated p1106 syndrome, ARHGEF1 deficiency, SEC61A1 deficiency, RAC2 deficiency, SH3KBP1 deficiency, CD20 deficiency, TACI deficiency, BAFF receptor deficiency, TWEAK deficiency, IRF2BP2 deficiency, CD19 deficiency, CD81 deficiency, CD21 deficiency, TRNT1 deficiency, NFKB1 deficiency, NKFB2 deficiency, IKAROS deficiency, ATP6AP1 gene, ATP6AP1 deficiency, and MOGS deficiency.

In some embodiments, the patient has mutations in one or more genes selected from PI3KCD (GOF), PIK3R1, PTEN, ARHGEF1, SH3KBP1, SEC61A1, RAC2, CD20, TAC1, TNFRSF13C, TWEAK, IRF2BP2, CD19, CD81, CD21, TRNT1, NFKB1, NFKB2, IKZF1, ATP6AP1, and MOGS.

In some embodiments, the PID is a disease or disorder associated with phagocyte deficiencies in a patient, such as congenital defects of phagocyte number, function, or both.

In some embodiments, the disease or disorder associated with phagocyte deficiencies (e.g., congenital defects of phagocyte number, function, or both) is selected from Shwachman-Diamond Sydrome, SRP54 deficiency, glycogen storage disease type 1i, Cohen syndrome, 3-Methylglutaconic aciduria, Barth Syndrome, Clericuzio syndrome, VPS45 deficiency, JAGN1 deficiency, WDR1 deficiency, SMARCD2 deficiency, specific granule deficiency, HYOU1 deficiency, P14/LAMTOR2 deficiency, Elastase deficiency, HAX1 deficiency, GFI 1 deficiency, G-CSF receptor deficiency and neutropenia with combined immune deficiency.

In some embodiments, the patient has mutations in one or more genes selected from DNAJC21, EFL1, SBDS, SRP54, G6PTI, COH1, CLPB, TAZ, C160RF57, VPS45, JAGN1, WDR1, SMARCD2, CEBPE, HYOU1, LAMTOR2, ELANE, HAX1, GFI1, CSF3R, and MKL1.

In some embodiments, the CXCR4 inhibitor is mavorixafor or a pharmaceutically acceptable salt thereof and the mavorixafor or pharmaceutically acceptable salt thereof is administered at a daily dose of from about 100 mg/day to about 600 mg/day; from about 200 mg/day to about 600 mg/day; from about 300 mg/day to about 500 mg/day; from about 350 mg/day to about 450 mg/day; or about 400 mg/day.

In some embodiments, the PID is selected from a common variable immune deficiency (CVID), a disease or disorder associated with CVID, or HAX1 deficiency.

In some embodiments, the CXCR4 inhibitor is mavorixafor or a pharmaceutically acceptable salt thereof and the mavorixafor or pharmaceutically acceptable salt thereof is administered at a daily dose of from about 100 mg/day to about 600 mg/day; from about 200 mg/day to about 600 mg/day; from about 300 mg/day to about 500 mg/day; from about 350 mg/day to about 450 mg/day; or about 400 mg/day.

### BRIEF DESCRIPTION OF THE FIGURES

FIG. 1A shows the CXCR4 expression levels in a human healthy donor (HD) vs. levels in human peripheral blood mononuclear cells (PBMCs) isolated from a patient with CVID due to pathogenic NFKB1 mutation (c.980dup p.Ala328Serfs*12). B cells from the CVID patient have increased CXCR4 expression compared to the healthy donor. FIG. 1B shows the degree of chemotaxis (% of input cells) of PBMCs from a CVID patient vs. PBMCs of a healthy donor in the presence of varying concentrations of CXCL12. B cells from the CVID patient have increased chemotaxis toward CXCL12 compared to healthy donor cells. FIG. 1C shows chemotaxis of PBMCs from a CVID patient vs. PBMCs from a healthy donor in the presence of varying concentrations of mavorixafor. Mavorixafor normalizes enhanced chemotaxis in CVID patient B cells.

### DETAILED DESCRIPTION OF CERTAIN EMBODIMENTS OF THE INVENTION

It has now been found that CXCR4 inhibitors such as mavorixafor (X4P-001) are useful for treating diseases and disorders related to immunodeficiencies, or diseases and disorders which present as imbalances in levels of one or more immune cells in a subject.

The CXCR4 inhibitor mavorixafor alone or in combination with other therapies is the first oral treatment to either acutely or chronically increase total peripheral WBCs 1.5- to 3-fold and WBC subsets across all disease populations examined, in both the presence (WHIM syndrome and WM) and absence (ccRCC and healthy volunteers) of CXCR4 gain-of-function mutation. Increases in WBC subsets occurred rapidly and were sustained during chronic treatment, with a larger treatment effect in patients with pre-existing cytopenia (WHIM syndrome) compared to patients without cytopenia at baseline (ccRCC and WM). Co-occurring reduction in infection burden was observed in the phase 2 trial in WHIM syndrome. Assessment of the beneficial effects of mavorixafor on total and WBC subsets is ongoing in a phase 3 trial of WHIM syndrome and a phase 1 trial of severe chronic neutropenia (SCN) that will assess the potential to correct cytopenias by elevating total WBC counts.

Accordingly, in one aspect, the present invention provides a method of correcting an imbalance of an immune cell population in a subject, comprising administering to the subject an effective amount of a CXCR4 inhibitor.

In some embodiments, the subject has a chronic immune cell imbalance. In some embodiments, the subject has an acute immune cell imbalance. In some embodiments, the immune cell imbalance is associated with a congenital primary immunodeficiency disease (PID). In some embodiments, the immune cell imbalance is associated with a disease state. In some embodiments, the disease state is cancer. In some embodiments, the cancer is renal cell carcinoma, clear cell renal cell carcinoma, papillary renal cancer, melanoma, pancreatic cancer, ovarian cancer, non-small cell lung cancer, Waldenstrom's macroglobulinemia (WM). In some embodiments, the cancer is a leukemia or lymphoma. In some embodiments, the PID is WHIM syndrome, chronic neutropenia or severe chronic neutropenia (SCN).

In some embodiments, the subject does not have a mutation in the subject's CXCR4 gene. In some embodiments, the subject does not have abnormal expression of CXCR4. In some embodiments, the subject's CXCR4 signaling is normal or within a normal range for a subject of the same sex and similar age and weight. In some embodiments, the subject's disease does not involve a mutation or gain-of-function in a CXCR4 gene. In some embodiments, the subject does not have a MYD88 or CXCR4 mutation. In some embodiments, the subject does not have WHIM syndrome, chronic neutropenia or severe chronic neutropenia (SCN).

The cells of the immune system can be categorized as lymphocytes (T-cells, B-cells and NK cells), neutrophils, and monocytes/macrophages. These are all types of white blood cells. The major proteins of the immune system are predominantly signaling proteins (often called cytokines), antibodies, and complement proteins.

In some embodiments, a method provided by the present invention corrects an imbalance in B-cells in the subject. B-cells (sometimes called B-lymphocytes) are specialized cells of the immune system whose major function is to produce antibodies (also called immunoglobulins or gamma-globulins). B-cells develop in the bone marrow from hematopoietic stem cells. As part of their maturation in the bone marrow, B-cells are trained or educated so that they do not produce antibodies to healthy tissues. When mature, B-cells can be found in the bone marrow, lymph nodes, spleen, some areas of the intestine, and the bloodstream.

In some embodiments, a method provided by the present invention corrects an imbalance in T-cells in the subject. T-cells (sometimes called T-lymphocytes and often named in lab reports as CD3 cells) directly attack cells infected with viruses, and they also act as regulators of the immune system. T-cells develop from hematopoietic stem cells in the bone marrow but complete their development in the thymus. The thymus is a specialized organ of the immune system in the chest. Within the thymus, immature lymphocytes develop into mature T-cells and T-cells with the potential to attack normal tissues are eliminated. The thymus is essential for this process, and T-cells cannot develop if the fetus does not have a thymus. Mature T-cells leave the thymus and populate other organs of the immune system, such as the spleen, lymph nodes, bone marrow and blood. Each T-cell reacts with a specific antigen, just as each antibody molecule reacts with a specific antigen.

T-cells have different abilities to recognize antigen and are varied in their function. There are “killer” or cytotoxic T-cells (often denoted in lab reports as CD8 T-cells), helper T-cells (often denoted in lab reports as CD4 T-cells), and regulatory T-cells. Each has a different role to play in the immune system. Killer, or cytotoxic, T-cells perform the actual destruction of infected cells. Killer T-cells protect the body from certain bacteria and viruses that have the ability to survive and even reproduce within the body's own cells. Killer T-cells also respond to foreign tissues in the body, such as a transplanted organ. The killer cell must migrate to the site of infection and directly bind to its target to ensure its destruction. Helper T-cells assist B-cells to produce antibodies and assist killer T-cells in their attack on foreign substances. Regulatory T-cells suppress or turn off other T-lymphocytes.

Natural killer (NK) cells are so named because they easily kill cells infected with viruses. They are said to be “natural killer” cells as they do not require the same thymic education that T-cells require. NK cells are derived from the bone marrow and are present in relatively low numbers in the bloodstream and in tissues. They are important in defending against viruses and possibly preventing cancer as well.

In some embodiments, a method provided by the present invention corrects an imbalance in neutrophils in the subject. Neutrophils or polymorphonuclear leukocytes (polys or PMN's) are the most numerous of all the types of white blood cells, making up about half or more of the total. They are also called granulocytes and appear on lab reports as part of a complete blood count (CBC with differential). They are found in the bloodstream and can migrate into sites of infection within a matter of minutes. These cells, like the other cells in the immune system, develop from hematopoietic stem cells in the bone marrow. Neutrophils increase in number in the bloodstream during infection and are in large part responsible for the elevated white blood cell count seen with some infections. They are capable of leaving the bloodstream and accumulating in tissues during the first few hours of an infection. Their major role is to ingest bacteria or fungi and kill them.

In some embodiments, a method provided by the present invention corrects an imbalance in monocytes (monocytopenia) in the subject. Monocytes are closely related to neutrophils and are found circulating in the bloodstream. They make up 5-10 percent of the white blood cells. They also line the walls of blood vessels in organs like the liver and spleen. Here they capture microorganisms in the blood as the microorganisms pass by. Monocytopenia is a reduction in blood monocyte count (ANC) to <500/mcL (<0.5×10⁹/L). Risk of certain infections is increased. It is diagnosed by complete blood count with differential. Typical treatment includes hematopoietic stem cell transplantation.

Macrophages are essential for killing fungi and certain bacteria. Macrophages live longer than neutrophils and are especially important for slow growing or chronic infections. Macrophages can be influenced by T-cells and often collaborate with T-cells in killing microorganisms.

In some embodiments, the subject has an imbalance of an immune cell population selected from T-cells, B-cells, NK cells, neutrophils, and monocytes. In some embodiments, the subject has leukopenia, neutropenia, or monocytopenia. In some embodiments, the subject exhibits a low total white blood cell (WBC) count.

In some embodiments, the patient has idiopathic neutropenia. In some embodiments, the patient has severe idiopathic neutropenia. In some embodiments, the patient has chronic neutropenia. In some embodiments, the patient has SCN, CIN, or AlN. In some embodiments, the patient has undergone genetic testing but no diagnosis of a genetic abnormality has been made. In some embodiments, the genetic testing was inconclusive. In some embodiments, the genetic testing revealed no known genetic abnormality, or a genetic abnormality not associated with neutropenia. In some embodiments, the patient has neutropenia not due to a genetic abnormality and due to one or more of an infectious, inflammatory, autoimmune, or malignant cause. In some embodiments, the malignant cause is a cancer.

In some embodiments, the patient has severe congenital neutropenia, suspected aplastic anemia, B-cell immunodeficiency, juvenile myelodysplastic syndrome (MDS), chronic myelomonocytic leukemia, a severe Epstein-Barr virus infection or Epstein-Barr-associated cancers, B-cell acute lymphoblastic leukemia, or unexplained bone marrow failure.

In some embodiments, the patient has undergone genetic testing and a genetic abnormality other than one associated with WHIM syndrome has been diagnosed. In some embodiments, the patient has a congenital neutropenia. In some embodiments, the patient has a genetic abnormality other than GSD1b, G6PC3 deficiency, GATA2 deficiency, and a genetically-defined condition without myeloid maturation arrest at the myelocyte/promyelocyte stage.

In some embodiments, the CXCR4 inhibitor is mavorixafor or a pharmaceutically acceptable salt thereof.

### CXCR4 Inhibitors

As described herein, a variety of CXCR4 inhibitors may be used in accordance with the present invention.

In some embodiments, the CXCR4 inhibitor is mavorixafor (X4P-001; AMD11070), or a pharmaceutically acceptable salt thereof.

In some embodiments, the CXCR4 inhibitor is one of those described in the following documents, or a pharmaceutically acceptable salt thereof: WO2017223229, WO2017223239, WO2017223243, WO2019126106, WO2020/264292, WO2003/022785, WO2003/055876, WO2004/106493, WO2004/091518, WO2004/093817, WO2006/049764, WO2005/090308, or WO2006/039250. Each of the foregoing documents is hereby incorporated by reference in its entirety.

In some embodiments, the CXCR4 inhibitor is AMD-12118, or a pharmaceutically acceptable salt thereof:

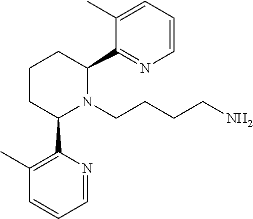 [SMILES: Cc1cccnc1[C@H]1CCC[C@@H](c2ncccc2C)N1CCCCN]

In some embodiments, the CXCR4 inhibitor is selected from one of the following, or a pharmaceutically acceptable salt thereof:

| STRUCTURE | MW | cLogP | PSA |
|---|---|---|---|
| 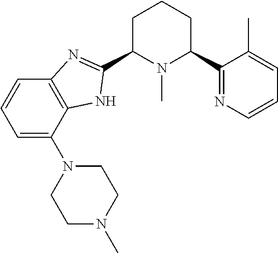 [SMILES: Cc1cccnc1[C@@H]1CCC[C@H](c2nc3cccc(N4CCN(C)CC4)c3[nH]2)N1C] | 404.6 | 3.3 | 46.5 |
| 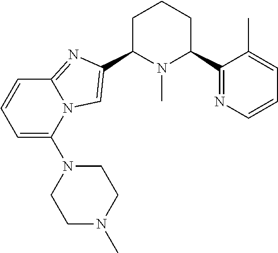 [SMILES: Cc1cccnc1[C@@H]1CCC[C@H](c2cn3c(N4CCN(C)CC4)cccc3n2)N1C] | 404.6 | 3.1 | 37.7 |
| 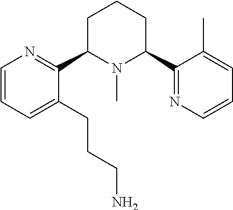 [SMILES: Cc1cccnc1[C@@H]1CCC[C@H](c2ncccc2CCCN)N1C] | 324.5 | 1.6 | 54.0 |
| 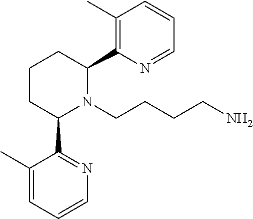 [SMILES: Cc1cccnc1[C@H]1CCC[C@@H](c2ncccc2C)N1CCCCN] | 338.5 | 2.1 | 54.0 |
| 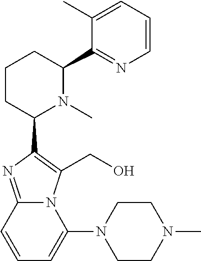 [SMILES: Cc1cccnc1[C@@H]1CCC[C@H](c2nc3cccc(N4CCN(C)CC4)n3c2CO)N1C] | 434.6 | 2.0 | 57.9 |
| 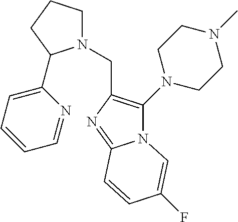 [SMILES: CN1CCN(c2c(CN3CCCC3c3ccccn3)nc3ccc(F)cn23)CC1] | 394.5 | 2.8 | 37.7 |
| 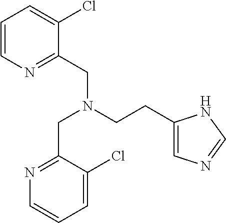 [SMILES: Clc1cccnc1CN(CCc1cnc[nH]1)Cc1ncccc1Cl] | 362.3 | 2.0 | 52.4 |
| 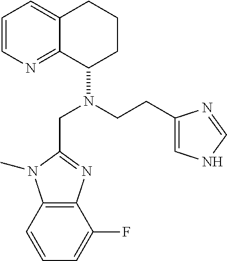 | 404.5 | 2.5 | 55.6 |
| 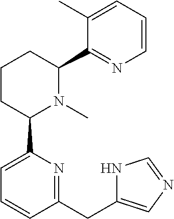 [SMILES: Cc1cccnc1[C@@H]1CCC[C@H](c2cccc(Cc3cnc[nH]3)n2)N1C] | 347.5 | 1.8 | 52.4 |
| 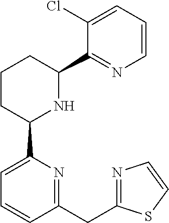 [SMILES: Clc1cccnc1[C@@H]1CCC[C@H](c2cccc(Cc3nccs3)n2)N1] | 370.9 | 2.3 | 49.1 |
| 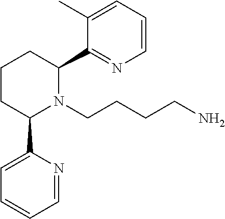 [SMILES: Cc1cccnc1[C@@H]1CCC[C@H](c2ccccn2)N1CCCCN] | 324.5 | 1.7 | 54.0 |

In some embodiments, the CXCR4 inhibitor is one of those described in Table 1, below. Each document listed in Table 1 is hereby incorporated by reference in its entirety.

TABLE 1
Exemplary CXCR4 Inhibitors

| MOLECULE/ | |
|---|---|
| COMPANY | STATUS/INFORMATION |
| Burixafor: TG-0054: Taigen Biotechnology, Inc. (2-{4-[6-amino-2- ({[(1,4r)-4-({[3- (cyclohexylamino) propyl] amino}methyl)cyclohexyl] methyl}amino)pyrimidin- 4-yl]piperazin-1- yl}ethyl)phosphonic acid | 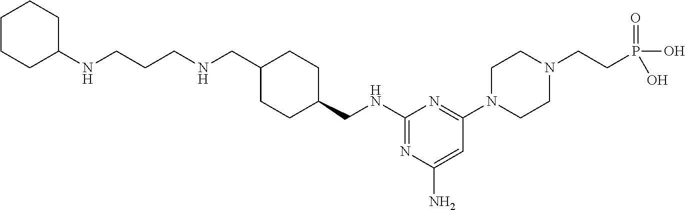 6/8/2014-ScinoPharm Taiwan, Ltd. (twse: 1789) specializing in the development and manufacture of active pharmaceutical ingredients, and TaiGen Biotechnology (4157.TW; F*TaiGen) jointly announced today the signing of a manufacturing contract for the clinical supply of the API of Burixafor. NCT00822341: Evaluating the Safety, Tolerability, Pharmacokinetics and Efficacy of TG-0054 With Single IV Doses Escalation in Healthy Volunteers |
| Bioline RX: BTK140 | Clinical trials HSC mobilization; Multiple Myeloma; T-ALL; |
| BL-8040 | Metastatic Pancreatic; Acute Myeloid Leukemia; Metastatic |
| Peptide [14-mer] | Pancreatic; Metastatic Gastric; Metastatic NSCLC. |
| | Beider et al. (2014) Mol Cancer Ther; 13(5); 1155-69. |
| | NCT02763384 BL-8040 and Nelarabine for Relapsed or |
| | Refractory T-Acute Lymphoblastic Leukemia/ Lymphoblastic |
| | Lymphoma: the addition of BL-8040 to nelarabine as a salvage |
| | therapy for patients with relapsed/refractory T-ALL/LBL will result |
| | in a higher CR rate than nelarabine alone without an increase in |
| | toxicity |
| | NCT01010880: Safety Study of a Chemokine Receptor (CXCR4) |
| | Antagonist in Multiple Myeloma Patients |
| Bristol Myers: | Ulocuplumab is a fully human IgG4 kappa anti-CXCR4 |
| Ulocuplumab; BMS- | monoclonal, being developed by Bristol-Myers Squibb as a potential |
| 936564 MDX-1338 | oncology therapeutic. Peptide sequences and structural details for |
| | this antibody are available from its IMGT/mAb-DB record. |
| | BLAST sequence analysis of the heavy and light chain peptide |
| | sequences of ulocuplumab, reveal identical matches with sequences |
| | claimed in patent US8450464. This identifies clone F7GL as the |
| | likely candidate for ulocuplumab. |
| | Kashyap et al. (2015) Oncotarget 7:2809-2822 |
| | NCT01359657 BMS-936564 Alone and in Combination With |
| | Lenalidomide (Revlimid)/Dexamethasone or Bortezomib |
| | (Velcade)/Dexamethasone in Relapsed/Refractory Multiple |
| | Myeloma |
| | NCT01120457: First in Human Study to Determine the Safety, |
| | Tolerability and Preliminary Efficacy of BMS-936564 in Subjects |
| | With Acute Myelogenous Leukemia and Selected B-cell Cancers |
| Proximagen Ltd: | Gravina et al. (2017) Journal of Hematology and Oncology 10: |
| PRX177561 | Brain-penetrating CXCR4 antagonist |
| Pfizer: PF-06747143 | Zhang et al. (2017) Scientific Reports; Targeting primary acute |
| | myeloid leukemia with a new CXCR4 antagonist IgG1 antibody |
| | (PF-06747143) Phase I trial |
| Polyphore: POL6326 | 5NCT01905475: CXCR4 Antagonism for Cell Mobilisation and |
| (balixafortide) | Healing in Acute Myocardial Infarction (CATCH-AMI) (CATCH- |
| | AMI) |
| | NCT01841476 Safety and Efficacy of POL6326 for Mobilization of |
| | Hematopoietic Stem Cells in Healthy Volunteers |
| CX549 (2S)-2-amino-5-[4-[[2- [[1-[3-[3- (cyclohexylamino) propylamino]propyl] triazol-4- yl]methylamino] quinazolin-4- yl]amino]piperidin-1- yl]-5-oxopentanoic acid | 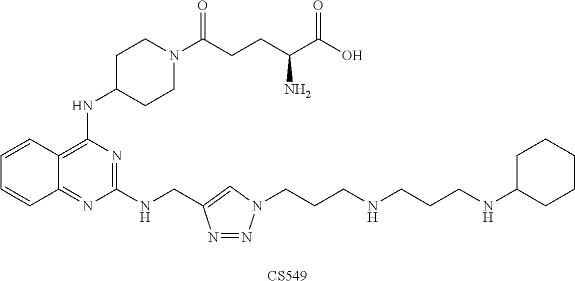 CX549 is a C-X-C chemokine receptor type 4 (CXCR4) antagonist [2], CXCR4 being the receptor for the chemokine CXCL12. CX549 is claimed as compound 63 in patent WO2016048861 |
| BPRCX807 | 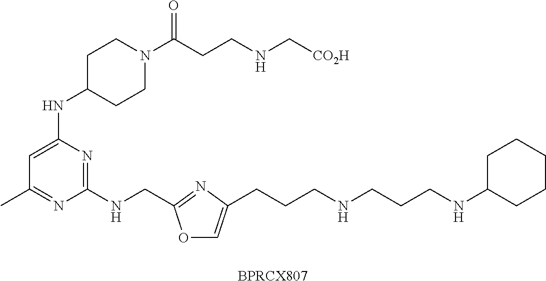 Song et al. (2021) PNAS 118:e2015433118 |
| LY2510924 Ei Lilly Cyclic Peptide | Cyclo[Phe-Tyr-Lys(iPr)-D-Arg-2-Nal-Gly-D-Glu]-Lys(iPr)-NH2 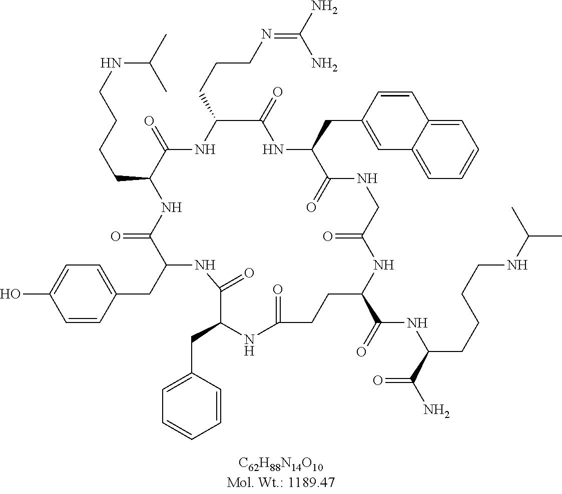 [SMILES: CC(C)NCCCC[C@H](NC(=O)[C@H]1CCC(=O)N[C@@H](Cc2ccccc2)C(=O)NC(Cc2ccc(O)cc2)C(=O)N[C@@H](CCCCNC(C)C)C(=O)N[C@H](CCCN=C(N)N)C(=O)N[C@@H](Cc2ccc3ccccc3c2)C(=O)NCC(=O)N1)C(N)=O] Peng et al (2015) Molecular Cellular Therapeutics; 14:480-490 NCT02737072: A Study of LY2510924 in Combination With Anti- PD-L1 Antibody, Durvalumab (MEDI4736) in Participants With Solid Tumors |
| TC14012 | Arg-Arg-Nal-Cys-Tyr-Cit-Lys-D-Cit-Pro-Tyr-Arg-Cit-Cys-Arg- |
| peptidomimetic | NH2 |
| | [RRACYXKXPYRXCR] |
| | Disulphide bond between cysteine residues at positions 4 and 14; |
| | amidation of C-terminal arginine residue; residue 3 is 3- |
| | napthylalanine (Nal); residues 6 and 8 are citruliline (Cit) and D- |
| | citruliline (D-Cit) (N5-aminocarbonylornithine) respectively |
| USL-311 Originally developed by Ligand; licensed to Usher- Smith And to Proximagen | 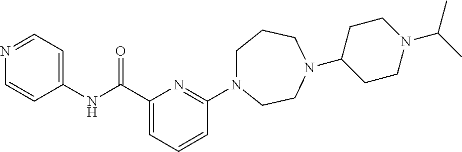 [SMILES: CC(C)N1CCC(N2CCCN(c3cccc(C(=O)Nc4ccncc4)n3)CC2)CC1] WO2018162924A1 NCT02765165: Phase 1/2 Study of USL311 Alone and in Combination With Lomustine in Subjects With Advanced Solid Tumors and Relapsed/Recurrent Glioblastoma Multiforme (GBM); USL311 as Single Agent and in Combination With Lomustine (CCNU) in Subjects With Advanced Solid Tumors |
| Adimab-Antibodies | US20100227774A1 |
| | Claim 1 claims an antibody that binds to loop 6 of human CXCR4. |
| FC131 | 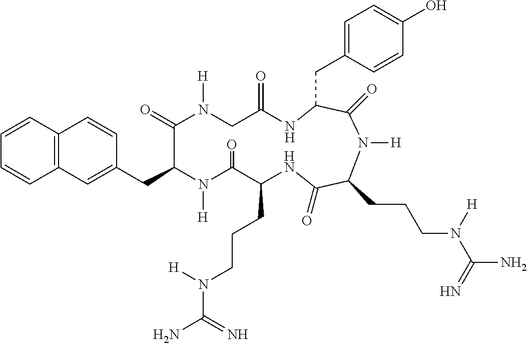 [SMILES: N=C(N)NCCC[C@@H]1NC(=O)[C@H](CCCNC(=N)N)NC(=O)[C@@H](Cc2ccc(O)cc2)NC(=O)CNC(=O)[C@H](Cc2ccc3ccccc3c2)NC1=O] |
| CTCE-9908 | a peptide antagonist |
| Chemokine | (amino acid sequence: KGVSLSYR-X-RYSLSVGK |
| Therapeutics | Faber et al. (2007) J. Biomedicine and Biotechnology 2007:Art. ID |
| Corporation | 26065 |
| GMI 1359 | WO 2016-089872 DISCLOSURE OF COMPOUNDS 9 and 16 |
| Glycomimetics | CXCR4i-E-Selectin antagonist |
| Heterobifunctional | Zhang et al. |
| Compounds | Steele, et al. CANCER RESEARCH (2015): 75(Supp) Abstract 425 |
| | Abstract 425: A small molecule glycomimetic antagonist of E- |
| | selectin and CXCR4 (GMI-1359) prevents pancreatic tumor |
| | metastasis and improves chemotherapy |
| REVIEWS | Tamamura et al. (2008)Perspectives in Medicinal Chemistry 2:1-9 |
| | Debnath et al. (2013) Theranostics 3:47-75 |
| | Li et al. (2018) Eur. J. Med Chem. 149:30-44 |

### Innate Immunity Defects—Susceptibility to Infections

Susceptibility to infections associated with innate immunity defects are treatable by the methods provided by the present invention.

In some embodiments, the present invention provides a method of treating predisposition to invasive bacterial infections (pyogenes), such as meningitis, sepsis, osteomyelitis, and/or abscesses, comprising administering to a subject in need thereof an effective amount of a CXCR4 inhibitor disclosed herein, or a pharmaceutically acceptable salt thereof.

In some embodiments, the present invention provides a method of treating IRAK4 deficiency, comprising administering to a subject in need thereof an effective amount of a CXCR4 inhibitor disclosed herein, or a pharmaceutically acceptable salt thereof. In some embodiments, the subject has a mutation in the IRAK4 gene. In some embodiments, the subject exhibits skin infections and/or upper respiratory tract infections. In some embodiments, the method corrects or treats skin infections and/or upper respiratory tract infections.

In some embodiments, the present invention provides a method of treating MyD88 deficiency, comprising administering to a subject in need thereof an effective amount of a CXCR4 inhibitor disclosed herein, or a pharmaceutically acceptable salt thereof. In some embodiments, the subject has a mutation in the MYD88 gene. In some embodiments, the subject exhibits skin infections and/or upper respiratory tract infections. In some embodiments, the method corrects or treats skin infections and/or upper respiratory tract infections.

In some embodiments, the subject does not have MyD88 deficiency and/or mutation in the MYD88X and/or CXCR4 genes, but the subject exhibits skin infections and/or upper respiratory tract infections. In some embodiments, the present invention provides a method, comprising administering to a subject in need thereof an effective amount of a CXCR4 inhibitor disclosed herein, or a pharmaceutically acceptable salt thereof, to correct or treat skin infections and/or upper respiratory tract infections, wherein the subject does not have mutation in the MYD88X and/or CXCR4 genes.

In some embodiments, the present invention provides a method of treating IRAK-1 deficiency, comprising administering to a subject in need thereof an effective amount of a CXCR4 inhibitor disclosed herein, or a pharmaceutically acceptable salt thereof. In some embodiments, the subject has a mutation in the IRAK1 gene. In some embodiments, the subject exhibits X-linked MECP2 deficiency-related syndrome. In some embodiments, the method corrects or treats X-linked MECP2 deficiency-related syndrome.

In some embodiments, the present invention provides a method of treating TIRAP deficiency or the effects of TIRAP mutations or dysfunction, comprising administering to a subject in need thereof an effective amount of a CXCR4 inhibitor disclosed herein, or a pharmaceutically acceptable salt thereof. In some embodiments, the subject has a mutation in the TIRAP gene. In some embodiments, the subject exhibits staphylococcal disease during childhood. In some embodiments, the method corrects or treats staphylococcal disease during childhood.

In some embodiments, the present invention provides a method of treating isolated congenital asplenia, comprising administering to a subject in need thereof an effective amount of a CXCR4 inhibitor disclosed herein, or a pharmaceutically acceptable salt thereof. In some embodiments, the subject has a mutation in the RPSA and/or HMOX genes. In some embodiments, the subject exhibits bacteremia (encapsulated bacteria), and/or no-spleen. In some embodiments, the method corrects or treats bacteremia (encapsulated bacteria), and/or no-spleen.

In some embodiments, the present invention provides a method of treating isolated congenital asplenia, comprising administering to a subject in need thereof an effective amount of a CXCR4 inhibitor disclosed herein, or a pharmaceutically acceptable salt thereof. In some embodiments, the subject has a mutation in the HMOX gene. In some embodiments, the subject exhibits hemolysis, nephritis, and/or inflammation. In some embodiments, the method corrects or treats hemolysis, nephritis, and/or inflammation.

In some embodiments, the present invention provides a method of treating predisposition to parasitic and fungal infections resulting from an innate immunity defect, comprising administering to a subject in need thereof an effective amount of a CXCR4 inhibitor disclosed herein, or a pharmaceutically acceptable salt thereof.

In some embodiments, the present invention provides a method of treating mucocutaneous candidiasis (CMC), comprising administering to a subject in need thereof an effective amount of a CXCR4 inhibitor disclosed herein, or a pharmaceutically acceptable salt thereof. In some embodiments, the subject exhibits chronic mucocutaneous candidiasis without ectodermal dysplasia. In some embodiments, the method corrects or treats chronic mucocutaneous candidiasis without ectodermal dysplasia.

In some embodiments, the present invention provides a method of treating STAT1 (GOF), comprising administering to a subject in need thereof an effective amount of a CXCR4 inhibitor disclosed herein, or a pharmaceutically acceptable salt thereof. In some embodiments, the subject has a mutation in the STAT1 gene. In some embodiments, the subject exhibits various fungal, bacterial and viral (such as HSV) infections, and/or cytopenias, and/or enteropathy. In some embodiments, the method corrects or treats various fungal, bacterial and viral (such as HSV) infections, and/or cytopenias), and/or enteropathy.

In some embodiments, the present invention provides a method of treating IL-17F deficiency or dysfunction, comprising administering to a subject in need thereof an effective amount of a CXCR4 inhibitor disclosed herein, or a pharmaceutically acceptable salt thereof. In some embodiments, the subject has a mutation in the IL17F gene. In some embodiments, the subject exhibits folliculitis. In some embodiments, the method corrects or treats folliculitis.

In some embodiments, the present invention provides a method of treating IL-17RA deficiency or dysfunction, comprising administering to a subject in need thereof an effective amount of a CXCR4 inhibitor disclosed herein, or a pharmaceutically acceptable salt thereof. In some embodiments, the subject has a mutation in the IL17RA gene. In some embodiments, the subject exhibits folliculitis, and/or susceptibility to S. aureus (skin infections). In some embodiments, the method corrects or treats folliculitis, and/or susceptibility to S. aureus (skin infections).

In some embodiments, the present invention provides a method of treating IL-17RC deficiency or dysfunction, comprising administering to a subject in need thereof an effective amount of a CXCR4 inhibitor disclosed herein, or a pharmaceutically acceptable salt thereof. In some embodiments, the subject has a mutation in the IL17RC gene. In some embodiments, the method corrects or treats symptoms associated with IL-17RC deficiency or dysfunction.

In some embodiments, the present invention provides a method of treating ACT1 deficiency, comprising administering to a subject in need thereof an effective amount of a CXCR4 inhibitor disclosed herein, or a pharmaceutically acceptable salt thereof. In some embodiments, the subject has a mutation in the ACT1 gene. In some embodiments, the subject exhibits blepharitis, folliculitis and/or macroglossia. In some embodiments, the method corrects or treats blepharitis, folliculitis and/or macroglossia.

In some embodiments, the present invention provides a method of treating CARD9 deficiency, comprising administering to a subject in need thereof an effective amount of a CXCR4 inhibitor disclosed herein, or a pharmaceutically acceptable salt thereof. In some embodiments, the subject has a mutation in the CARD9 gene. In some embodiments, the subject exhibits predisposition to invasive fungal diseases, such as, invasive candidiasis infection, and/or deep dermatophytoses. In some embodiments, the method corrects or treats predisposition to invasive fungal diseases, such as invasive candidiasis infection, and/or deep dermatophytoses.

In some embodiments, the present invention provides a method of treating trypanosomiasis, comprising administering to a subject in need thereof an effective amount of a CXCR4 inhibitor disclosed herein, or a pharmaceutically acceptable salt thereof. In some embodiments, the subject has a mutation in the APOL1 gene. In some embodiments, the subject exhibits trypanosomiasis. In some embodiments, the method corrects or treats trypanosomiasis.

In some embodiments, the present invention provides a method of treating osteopetrosis, comprising administering to a subject in need thereof an effective amount of a CXCR4 inhibitor disclosed herein, or a pharmaceutically acceptable salt thereof. In some embodiments, the subject has a mutation in the TNFRSF11A, PLEKHM1, and/or TCIRG1 genes. In some embodiments, the subject exhibits hypocalcemia. In some embodiments, the method corrects or treats hypocalcemia. In some embodiments, the subject has a mutation in the CLCN7, and/or OSTM1 genes. In some embodiments, the subject exhibits hypocalcemia, and/or neurologic features. In some embodiments, the method corrects or treats hypocalcemia, and/or neurologic features. In some embodiments, the subject has a mutation in the TNFSF11 gene. In some embodiments, the subject exhibits severe growth retardation. In some embodiments, the method corrects or treats severe growth retardation.

In some embodiments, the present invention provides a method of treating hidradenitis suppurativa, comprising administering to a subject in need thereof an effective amount of a CXCR4 inhibitor disclosed herein, or a pharmaceutically acceptable salt thereof. In some embodiments, the subject has a mutation in the PSENEN and/or NCSTN genes. In some embodiments, the subject exhibits acne. In some embodiments, the method corrects or treats acne. In some embodiments, the subject has a mutation in the PSEN genes. In some embodiments, the subject exhibits hyperpigmentation. In some embodiments, the method corrects or treats hyperpigmentation.

In some embodiments, the present invention provides a method of treating acute liver failure due to NBAS deficiency, comprising administering to a subject in need thereof an effective amount of a CXCR4 inhibitor disclosed herein, or a pharmaceutically acceptable salt thereof. In some embodiments, the subject has a mutation in the NBAS gene. In some embodiments, the subject exhibits fever induced liver failure. In some embodiments, the method corrects or treats fever induced liver failure.

In some embodiments, the present invention provides a method of treating acute necrotizing encephalopathy, comprising administering to a subject in need thereof an effective amount of a CXCR4 inhibitor disclosed herein, or a pharmaceutically acceptable salt thereof. In some embodiments, the subject has a mutation in the RANBP2 gene. In some embodiments, the subject exhibits fever induced acute encephalopathy. In some embodiments, the method corrects or treats fever induced acute encephalopathy.

In some embodiments, the present invention provides a method of treating IRF4 haploinsufficiency, comprising administering to a subject in need thereof an effective amount of a CXCR4 inhibitor disclosed herein, or a pharmaceutically acceptable salt thereof. In some embodiments, the subject has a mutation in the IRF4 gene. In some embodiments, the subject exhibits Whipple's disease. In some embodiments, the method corrects or treats Whipple's disease.

In some embodiments, the present invention provides a method of treating severe phenotypes of Mendelian susceptibility to mycobacterial disease (MSMD), comprising administering to a subject in need thereof an effective amount of a CXCR4 inhibitor disclosed herein, or a pharmaceutically acceptable salt thereof.

In some embodiments, the present invention provides a method of treating complete IFNGR1 deficiency, and/or IFNGR2 deficiency, comprising administering to a subject in need thereof an effective amount of a CXCR4 inhibitor disclosed herein, or a pharmaceutically acceptable salt thereof. In some embodiments, the subject has a mutation in the IFNGR1 and/or IFNGR2 genes. In some embodiments, the subject exhibits serious disseminated BCG and environmental mycobacterial infections of soft tissue, bone marrow, lungs, skin, bones, and lymph nodes. In some embodiments, the method corrects or treats serious disseminated BCG and environmental mycobacterial infections of soft tissue, bone marrow, lungs, skin, bones, and lymph nodes. In some embodiments, the subject exhibits infections of Salmonella spp., Listeria monocytogenes and/or viruses. In some embodiments, the method corrects or treats infections of Salmonella spp., Listeria monocytogenes and/or viruses.

In some embodiments, the present invention provides a method of treating partial IFNGR1 deficiency, comprising administering to a subject in need thereof an effective amount of a CXCR4 inhibitor disclosed herein, or a pharmaceutically acceptable salt thereof.

In some embodiments, the present invention provides a method of treating moderate phenotypes of Mendelian susceptibility to mycobacterial disease (MSMD), comprising administering to a subject in need thereof an effective amount of a CXCR4 inhibitor disclosed herein, or a pharmaceutically acceptable salt thereof.

In some embodiments, the present invention provides a method of treating IL-12 and IL-23 receptor b1 chain deficiency, comprising administering to a subject in need thereof an effective amount of a CXCR4 inhibitor disclosed herein, or a pharmaceutically acceptable salt thereof. In some embodiments, the subject has a mutation in the IL12RB1 gene.

In some embodiments, the present invention provides a method of treating IL-12 receptor b1 chain deficiency, comprising administering to a subject in need thereof an effective amount of a CXCR4 inhibitor disclosed herein, or a pharmaceutically acceptable salt thereof. In some embodiments, the subject has a mutation in the IL12RB1 gene.

In some embodiments, the present invention provides a method of treating IL-23 receptor b1 chain deficiency, comprising administering to a subject in need thereof an effective amount of a CXCR4 inhibitor disclosed herein, or a pharmaceutically acceptable salt thereof. In some embodiments, the subject has a mutation in the IL12RB1 gene.

In some embodiments, the present invention provides a method of treating IL-12p40 (IL-12 and IL-2) deficiency, comprising administering to a subject in need thereof an effective amount of a CXCR4 inhibitor disclosed herein, or a pharmaceutically acceptable salt thereof. In some embodiments, the subject has a mutation in the IL12B gene.

In some embodiments, the present invention provides a method of treating IL-12Rb2 deficiency, comprising administering to a subject in need thereof an effective amount of a CXCR4 inhibitor disclosed herein, or a pharmaceutically acceptable salt thereof. In some embodiments, the subject has a mutation in the IL12RB2 gene.

In some embodiments, the present invention provides a method of treating IL-23R deficiency, comprising administering to a subject in need thereof an effective amount of a CXCR4 inhibitor disclosed herein, or a pharmaceutically acceptable salt thereof. In some embodiments, the subject has a mutation in the IL23R gene.

In some embodiments, the present invention provides a method of treating STAT1 (LOF), comprising administering to a subject in need thereof an effective amount of a CXCR4 inhibitor disclosed herein, or a pharmaceutically acceptable salt thereof. In some embodiments, the subject has a mutation in the STAT1 gene.

In some embodiments, the present invention provides a method of treating partial IFNγR1 deficiency, comprising administering to a subject in need thereof an effective amount of a CXCR4 inhibitor disclosed herein, or a pharmaceutically acceptable salt thereof. In some embodiments, the subject has a mutation in the IFNGR1 gene.

In some embodiments, the present invention provides a method of treating partial IFNγR2 deficiency, comprising administering to a subject in need thereof an effective amount of a CXCR4 inhibitor disclosed herein, or a pharmaceutically acceptable salt thereof. In some embodiments, the subject has a mutation in the IFNGR2 gene.

In some embodiments, the present invention provides a method of treating AD IFNGR1, comprising administering to a subject in need thereof an effective amount of a CXCR4 inhibitor disclosed herein, or a pharmaceutically acceptable salt thereof. In some embodiments, the subject has a mutation in the IFNGR1 gene. In some embodiments, the subject exhibits mycobacterial osteomyelitis. In some embodiments, the method corrects or treats mycobacterial osteomyelitis.

In some embodiments, the present invention provides a method of treating partial SPPL2a deficiency, comprising administering to a subject in need thereof an effective amount of a CXCR4 inhibitor disclosed herein, or a pharmaceutically acceptable salt thereof. In some embodiments, the subject has a mutation in the SPPL2A gene.

In some embodiments, the present invention provides a method of treating partial Tyk2 deficiency, comprising administering to a subject in need thereof an effective amount of a CXCR4 inhibitor disclosed herein, or a pharmaceutically acceptable salt thereof. In some embodiments, the subject has a mutation in the TYK2 gene. In some embodiments, the subject exhibits susceptibility to viruses, +/−elevated IgE, multiple cytokine signaling defect, P1104A TYK2 homozygosity, MSMD, and/or tuberculosis. In some embodiments, the method corrects or treats susceptibility to viruses, +/−elevated IgE, multiple cytokine signaling defect, P1104A TYK2 homozygosity, MSMD, and/or tuberculosis.

In some embodiments, the present invention provides a method of treating macrophage gp91 phox deficiency, comprising administering to a subject in need thereof an effective amount of a CXCR4 inhibitor disclosed herein, or a pharmaceutically acceptable salt thereof. In some embodiments, the subject has a mutation in the CYBB gene.

In some embodiments, the present invention provides a method of treating IRF8 deficiency, comprising administering to a subject in need thereof an effective amount of a CXCR4 inhibitor disclosed herein, or a pharmaceutically acceptable salt thereof. In some embodiments, the subject has a mutation in the IRF8X gene. In some embodiments, the subject exhibits multiple other infectious agents and/or myeloproliferation. In some embodiments, the method corrects or treats multiple other infectious agents and/or myeloproliferation.

In some embodiments, the present invention provides a method of treating IFG15 deficiency, comprising administering to a subject in need thereof an effective amount of a CXCR4 inhibitor disclosed herein, or a pharmaceutically acceptable salt thereof. In some embodiments, the subject has a mutation in the ISG15 gene. In some embodiments, the subject exhibits brain calcification, and/or IFNg production defect. In some embodiments, the method corrects or treats brain calcification, and/or IFNg production defect.

In some embodiments, the present invention provides a method of treating RORγT deficiency, comprising administering to a subject in need thereof an effective amount of a CXCR4 inhibitor disclosed herein, or a pharmaceutically acceptable salt thereof. In some embodiments, the subject has a mutation in the RORC gene. In some embodiments, the subject exhibits susceptibility to Candida, IFNg production defect, and/or complete absence of IL-17A/F-producing T-cells. In some embodiments, the method corrects or treats susceptibility to Candida, IFNg production defect, and/or complete absence of IL-17A/F-producing T-cells.

In some embodiments, the present invention provides a method of treating JAK1 (LOF), comprising administering to a subject in need thereof an effective amount of a CXCR4 inhibitor disclosed herein, or a pharmaceutically acceptable salt thereof. In some embodiments, the subject has a mutation in the JAK1 gene. In some embodiments, the subject exhibits susceptibility to viruses, urothelial carcinoma, and/or IFNg production. In some embodiments, the method corrects or treats susceptibility to viruses, urothelial carcinoma, and/or IFNg production.

In some embodiments, the present invention provides a method of treating epidermodysplasia verruciformis (HPV), comprising administering to a subject in need thereof an effective amount of a CXCR4 inhibitor disclosed herein, or a pharmaceutically acceptable salt thereof. In some embodiments, the subject exhibits infections and/or cancer of the skin. In some embodiments, the method corrects or treats infections and/or cancer of the skin.

In some embodiments, the present invention provides a method of treating partial EVER1 deficiency, comprising administering to a subject in need thereof an effective amount of a CXCR4 inhibitor disclosed herein, or a pharmaceutically acceptable salt thereof. In some embodiments, the subject has a mutation in the TMC6 gene.

In some embodiments, the present invention provides a method of treating partial EVER2 deficiency, comprising administering to a subject in need thereof an effective amount of a CXCR4 inhibitor disclosed herein, or a pharmaceutically acceptable salt thereof. In some embodiments, the subject has a mutation in the TMC8 gene.

In some embodiments, the present invention provides a method of treating partial CIB1 deficiency, comprising administering to a subject in need thereof an effective amount of a CXCR4 inhibitor disclosed herein, or a pharmaceutically acceptable salt thereof. In some embodiments, the subject has a mutation in the CIB1 gene.

In some embodiments, the present invention provides a method of treating WHIM (warts, hypogammaglobulinemia, infections, myelokathexis), comprising administering to a subject in need thereof an effective amount of a CXCR4 inhibitor disclosed herein, or a pharmaceutically acceptable salt thereof. In some embodiments, the subject has a gain-of-function (GOF) mutation in the CXCR4 gene. In some embodiments, the subject exhibits warts (HPV) infection, neutropenia, low B cell number, and/or hypogammaglobulinemia. In some embodiments, the method corrects or treats warts (HPV) infection, neutropenia, low B cell number, and/or hypogammaglobulinemia.

In some embodiments, the present invention provides a method of treating predisposition to severe viral infection, comprising administering to a subject in need thereof an effective amount of a CXCR4 inhibitor disclosed herein, or a pharmaceutically acceptable salt thereof.

In some embodiments, the present invention provides a method of treating STAT1 deficiency, comprising administering to a subject in need thereof an effective amount of a CXCR4 inhibitor disclosed herein, or a pharmaceutically acceptable salt thereof. In some embodiments, the subject has a mutation in the STAT1 gene. In some embodiments, the subject exhibits mycobacterial infections. In some embodiments, the method corrects or treats mycobacterial infections.

In some embodiments, the present invention provides a method of treating STAT2 deficiency, comprising administering to a subject in need thereof an effective amount of a CXCR4 inhibitor disclosed herein, or a pharmaceutically acceptable salt thereof. In some embodiments, the subject has a mutation in the STAT2 gene. In some embodiments, the subject exhibits disseminated vaccine-strain measles. In some embodiments, the method corrects or treats disseminated vaccine-strain measles.

In some embodiments, the present invention provides a method of treating IRF7 deficiency, comprising administering to a subject in need thereof an effective amount of a CXCR4 inhibitor disclosed herein, or a pharmaceutically acceptable salt thereof. In some embodiments, the subject has a mutation in the IRF7 gene. In some embodiments, the subject exhibits severe influenza disease. In some embodiments, the method corrects or treats severe influenza disease.

In some embodiments, the present invention provides a method of treating IRF9 deficiency, comprising administering to a subject in need thereof an effective amount of a CXCR4 inhibitor disclosed herein, or a pharmaceutically acceptable salt thereof. In some embodiments, the subject has a mutation in the IRF9 gene. In some embodiments, the subject exhibits severe influenza disease. In some embodiments, the method corrects or treats severe influenza disease.

In some embodiments, the present invention provides a method of treating IFNAR1 deficiency, comprising administering to a subject in need thereof an effective amount of a CXCR4 inhibitor disclosed herein, or a pharmaceutically acceptable salt thereof. In some embodiments, the subject has a mutation in the IFNAR1 gene. In some embodiments, the subject exhibits severe disease caused by yellow fever vaccine and/or measles vaccine. In some embodiments, the method corrects or treats severe disease caused by yellow fever vaccine and/or measles vaccine.

In some embodiments, the present invention provides a method of treating IFNAR2 deficiency, comprising administering to a subject in need thereof an effective amount of a CXCR4 inhibitor disclosed herein, or a pharmaceutically acceptable salt thereof. In some embodiments, the subject has a mutation in the IFNAR2 gene. In some embodiments, the subject exhibits disseminated vaccine-strain measles, human herpesvirus 6 (HHV6), and/or no response to IFN-α. In some embodiments, the method corrects or treats disseminated vaccine-strain measles, human herpesvirus 6 (HHV6), and/or no response to IFN-α.

In some embodiments, the present invention provides a method of treating CD16 deficiency, comprising administering to a subject in need thereof an effective amount of a CXCR4 inhibitor disclosed herein, or a pharmaceutically acceptable salt thereof. In some embodiments, the subject has a mutation in the FCGR3A gene. In some embodiments, the subject exhibits severe herpes viral infections, VZV, Epstein Barr virus (EBV), and/or HPV. In some embodiments, the method corrects or treats severe herpes viral infections, VZV, Epstein Barr virus (EBV), and/or HPV.

In some embodiments, the present invention provides a method of treating MDAS deficiency, comprising administering to a subject in need thereof an effective amount of a CXCR4 inhibitor disclosed herein, or a pharmaceutically acceptable salt thereof. In some embodiments, the subject has a mutation in the IFIH1 gene. In some embodiments, the subject exhibits rhinovirus and/or other RNA viruses. In some embodiments, the method corrects or treats rhinovirus and/or other RNA viruses

In some embodiments, the present invention provides a method of treating RNA polymerase III deficiency, comprising administering to a subject in need thereof an effective amount of a CXCR4 inhibitor disclosed herein, or a pharmaceutically acceptable salt thereof. In some embodiments, the subject has mutations in the POLR3A, POLR3C, and/or POLR3F genes. In some embodiments, the subject exhibits severe VZV infection. In some embodiments, the method corrects or treats severe VZV infection.

In some embodiments, the present invention provides a method of treating IL-18BP deficiency, comprising administering to a subject in need thereof an effective amount of a CXCR4 inhibitor disclosed herein, or a pharmaceutically acceptable salt thereof. In some embodiments, the subject has a mutation in the IL18BP gene. In some embodiments, the subject exhibits fulminant viral hepatitis. In some embodiments, the method corrects or treats fulminant viral hepatitis.

In some embodiments, the present invention provides a method of treating herpes simplex encephalitis (HSE), comprising administering to a subject in need thereof an effective amount of a CXCR4 inhibitor disclosed herein, or a pharmaceutically acceptable salt thereof. In some embodiments, the subject has mutations in the UNC93B1, TRAF3, TICAM1, TBK1, and/or IRF3 genes. In some embodiments, the subject exhibits HSE during primary infection with herpes simplex virus type 1 (HSV1). In some embodiments, the method corrects or treats HSE during primary infection with HSV1. In some embodiments, the subject has mutation in the TLR3 gene. In some embodiments, the subject exhibits HSE during primary infection with herpes simplex virus type 1 (HSV1), severe pulmonary influenza, and/or VZV. In some embodiments, the method corrects or treats HSE during primary infection with herpes simplex virus type 1 (HSV1), severe pulmonary influenza, and/or VZV. In some embodiments, the subject has mutation in the DBR1 gene. In some embodiments, the subject exhibits HSE during primary infection with herpes simplex virus type 1 (HSV1), and/or other viral infections of the brainstem. In some embodiments, the method corrects or treats HSE during primary infection with herpes simplex virus type 1 (HSV1), and/or other viral infections of the brainstem.

Primary Immunodeficiencies with Functional Defects of Phagocytes

Various primary immunodeficiency diseases and disorders with functional defects of phagocytes are treatable by the methods provided by the present invention.

In some embodiments, the present invention provides a method of treating GATA2 deficiency, comprising administering to a subject in need thereof an effective amount of a CXCR4 inhibitor disclosed herein, or a pharmaceutically acceptable salt thereof. In some embodiments, the subject has a mutation in the GATA2 gene. In some embodiments, the subject exhibits susceptibility to mycobacteria, papilloma viruses, histoplasmosis, lymphedema, alveolar proteinosis, myelodysplasia/AML/CMML (chronic myelomonocytic leukemia), multi lineage cytopenias and/or low NK. In some embodiments, the method corrects or treats susceptibility to mycobacteria, papilloma viruses, histoplasmosis, lymphedema, alveolar proteinosis, myelodysplasia/AML/CMML (chronic myelomonocytic leukemia), multi lineage cytopenias and/or low NK.

In some embodiments, the subject does not have GATA2 deficiency, and/or mutation in the GATA2 gene, but the subject exhibits susceptibility to mycobacteria, papilloma viruses, histoplasmosis, lymphedema, alveolar proteinosis, myelodysplasia/AML/CMML (chronic myelomonocytic leukemia), multi lineage cytopenias and/or low NK. In some embodiments, the present invention provides a method, comprising administering to a subject in need thereof an effective amount of a CXCR4 inhibitor disclosed herein, or a pharmaceutically acceptable salt thereof, to correct or treat susceptibility to mycobacteria, papilloma viruses, histoplasmosis, lymphedema, alveolar proteinosis, myelodysplasia/AML/CMML (chronic myelomonocytic leukemia), multi lineage cytopenias and/or low NK, wherein the subject does not have GATA2 deficiency, and/or mutation in the GATA2 gene.

In some embodiments, the patient does not exhibit idiopathic CD+ lymphocytopenia (ICL). In some embodiments, the patient does not have Wiskott-Aldrich Syndrome.

In some embodiments, the present invention provides a method of treating Leukocyte Adhesion Deficiency Type 1 (LAD1), comprising administering to a subject in need thereof an effective amount of a CXCR4 inhibitor disclosed herein, or a pharmaceutically acceptable salt thereof. In some embodiments, the subject has a mutation in the ITGB2 gene. In some embodiments, the subject exhibits defective or deficient beta-2 integrin. In some embodiments, the method corrects or treats defective or deficient beta-2 integrin.

In some embodiments, the present invention provides a method of treating Leukocyte Adhesion Deficiency Type 2 (LAD2), comprising administering to a subject in need thereof an effective amount of a CXCR4 inhibitor disclosed herein, or a pharmaceutically acceptable salt thereof. In some embodiments, the subject has a mutation in the SLC35C1 gene. In some embodiments, the subject exhibits abnormal metabolism of fucose or absence of Sialyl Lewis X of E-selectin. In some embodiments, the method corrects or treats absence of Sialyl Lewis X of E-selectin.

In some embodiments, the present invention provides a method of treating Leukocyte Adhesion Deficiency Type 3 (LAD3), comprising administering to a subject in need thereof an effective amount of a CXCR4 inhibitor disclosed herein, or a pharmaceutically acceptable salt thereof. In some embodiments, the subject has a mutation in the FERMT3 gene. In some embodiments, the subject exhibits impaired integrin activation cascade. In some embodiments, the method corrects or treats absence of impaired integrin activation cascade.

In some embodiments, the present invention provides a method of treating pulmonary alveolar proteinosis, comprising administering to a subject in need thereof an effective amount of a CXCR4 inhibitor disclosed herein, or a pharmaceutically acceptable salt thereof. In some embodiments, the subject has a mutation in the CSF2RA and/or CSF2RB genes. In some embodiments, the subject exhibits affected alveolar macrophages and/or affected GM-CSF signaling. In some embodiments, the method corrects or treats affected alveolar macrophages and/or affected GM-CSF signaling.

In some embodiments, the present invention provides a method of treating chronic granulomatous disease (CGD), comprising administering to a subject in need thereof an effective amount of a CXCR4 inhibitor disclosed herein, or a pharmaceutically acceptable salt thereof. In some embodiments, the subject has a mutation in the NCF1, CYBA, NCF4, NCF2 and/or CYBC1 genes. In some embodiments, the subject exhibits early onset of severe and recurrent infections affecting initially the natural barriers of the patient (lungs, lymph nodes, skin), and eventually inner structures (liver, spleen, bones, brain, and hepatic abscess). Infecting pathogens include, but are not limited to, catalase negative bacteria (S. aureus and gram-negative bacilli, Aspergillus, Candida), Burkholderia cencia, Chrormobacterium violaceum, Nocardia, and invasive Serratia marcescens. In some embodiments, the method corrects or treats above-mentioned early onset of severe and recurrent infections. In some embodiments, the subject exhibits autoinflammatory phenotype, IBD (Crohn's like disease), and/or perianal disease. In some embodiments, the method corrects or treats autoinflammatory phenotype, IBD (Crohn's like disease), and/or perianal disease. In some embodiments, the subject exhibits granulomas obstructing respiratory, urinary and/or gastrointestinal tracts. In some embodiments, the method corrects or treats granulomas obstructing respiratory, urinary and/or gastrointestinal tracts.

In some embodiments, the present invention provides a method of treating Rac-2 deficiency, comprising administering to a subject in need thereof an effective amount of a CXCR4 inhibitor disclosed herein, or a pharmaceutically acceptable salt thereof. In some embodiments, the subject has a mutation in the RAC2 gene. In some embodiments, the subject exhibits poor wound healing, and/or LAD phenotype (leukocytosis). In some embodiments, the method corrects or treats poor wound healing, and/or LAD phenotype (leukocytosis).

In some embodiments, the present invention provides a method of treating G6PD deficiency Class 1, comprising administering to a subject in need thereof an effective amount of a CXCR4 inhibitor disclosed herein, or a pharmaceutically acceptable salt thereof. In some embodiments, the subject has a mutation in the G6PD gene. In some embodiments, the subject exhibits infections. In some embodiments, the method corrects or treats infections.

### DiGeorge Syndrome and Other Primary Immunodeficiency Diseases and

In some embodiments, the present invention provides a method of treating thymic defects, which may be accompanied by additional congenital anomalies, such as DiGeorge Syndrome and CHARGE syndrome, comprising administering to a subject in need thereof an effective amount of a CXCR4 inhibitor disclosed herein, or a pharmaceutically acceptable salt thereof.

In some embodiments, the present invention provides a method of treating DiGeorge Syndrome (DGS), comprising administering to a subject in need thereof an effective amount of a CXCR4 inhibitor disclosed herein, or a pharmaceutically acceptable salt thereof. In some embodiments, the subject has a mutation in the TBX1 gene. In some embodiments, the subject exhibits hypoparathyroidism, conotruncal cardiac malformation, velopalatal insufficiency, facial dysmorphism, and/or intellectual disability. In some embodiments, the method corrects or treats hypoparathyroidism, conotruncal cardiac malformation, velopalatal insufficiency, facial dysmorphism, and/or intellectual disability. In some embodiments, the subject exhibits decreased Ig levels, low T-cells and/or low levels of T-cell receptor excision circle (TRECs) at newborn screening (NBS). In some embodiments, the method corrects or treats decreased Ig levels, low T-cells and/or low levels of TRECs at NBS.

In some embodiments, the present invention provides a method of treating CHARGE syndrome, comprising administering to a subject in need thereof an effective amount of a CXCR4 inhibitor disclosed herein, or a pharmaceutically acceptable salt thereof. In some embodiments, the subject has mutations in the CHD7 and/or SEMA3E genes. In some embodiments, the subject exhibits coloboma heart anomaly, choanal atresia, intellectual disability, genital and ear anomalies, and/or CNS malformations. In some embodiments, the method corrects or treats coloboma heart anomaly, choanal atresia, intellectual disability, genital and ear anomalies, and/or CNS malformations. In some embodiments, the subject exhibits SCID-like low TRECs, decreased Ig, decreased T-cells and/or decreased response to phytohemagglutinin (PHA). In some embodiments, the method corrects or treats SCID-like low TRECs, decreased Ig, decreased T-cells and/or decreased response to PHA.

In some embodiments, the present invention provides a method of treating Chromosome 10p13-p14 deletion syndrome, comprising administering to a subject in need thereof an effective amount of a CXCR4 inhibitor disclosed herein, or a pharmaceutically acceptable salt thereof. In some embodiments, the subject has a mutation in the 10p13-p14DS gene. In some embodiments, the subject exhibits hypoparathyroidism, renal disease, deafness, growth retardation, facial dysmorphism, and/or cardiac defects. In some embodiments, the method corrects or treats hypoparathyroidism, renal disease, deafness, growth retardation, facial dysmorphism, and/or cardiac defects.

In some embodiments, the present invention provides a method of treating Jacobsen syndrome, comprising administering to a subject in need thereof an effective amount of a CXCR4 inhibitor disclosed herein, or a pharmaceutically acceptable salt thereof. In some embodiments, the subject has a mutation in the 11q23DEL gene. In some embodiments, the subject exhibits recurrent respiratory infections, multiple warts, facial dysmorphism, growth retardation, lymphopenia, hypogammaglobulinemia, low NK cells, low B-cells, and/or low switched memory B-cells. In some embodiments, the method corrects or treats recurrent respiratory infections, multiple warts, facial dysmorphism, growth retardation, lymphopenia, hypogammaglobulinemia, low NK cells, low B-cells, and/or low switched memory B-cells.

In some embodiments, the present invention provides a method of treating FOXN1 haploinsufficiency, comprising administering to a subject in need thereof an effective amount of a CXCR4 inhibitor disclosed herein, or a pharmaceutically acceptable salt thereof. In some embodiments, the subject has a mutation in the FOXN1 gene. In some embodiments, the subject exhibits recurrent viral and bacterial respiratory tract infections, eczema, dermatitis, nail dystrophy, and/or T-cell lymphopenia. In some embodiments, the method corrects or treats recurrent viral and bacterial respiratory tract infections, eczema, dermatitis, nail dystrophy, and/or T-cell lymphopenia.

In some embodiments, the present invention provides a method of treating immuno-osseous dysplasias, such as cartilage hair hypoplasia, Schimke Syndrome, MOPD1 deficiency, MYSM1 deficiency and EXTL3 deficiency, comprising administering to a subject in need thereof an effective amount of a CXCR4 inhibitor disclosed herein, or a pharmaceutically acceptable salt thereof.

In some embodiments, the present invention provides a method of treating cartilage hair hypoplasia, comprising administering to a subject in need thereof an effective amount of a CXCR4 inhibitor disclosed herein, or a pharmaceutically acceptable salt thereof. In some embodiments, the subject has a mutation in the RMRP gene. In some embodiments, the subject exhibits short-limbed dwarfism with metaphyseal dysostosis, sparse hair, bone marrow failure, autoimmunity, susceptibility to lymphoma and other cancers, impaired spermatogenesis, neuronal dysplasia of the intestine, severe combined immunodeficiency (SCID), impaired lymphocyte proliferation, low Ig levels, and/or low T-cell levels. In some embodiments, the method corrects or treats short-limbed dwarfism with metaphyseal dysostosis, sparse hair, bone marrow failure, autoimmunity, susceptibility to lymphoma and other cancers, impaired spermatogenesis, neuronal dysplasia of the intestine, SCID, impaired lymphocyte proliferation, low Ig levels, and/or low T-cell levels.

In some embodiments, the present invention provides a method of treating Schimke Syndrome, comprising administering to a subject in need thereof an effective amount of a CXCR4 inhibitor disclosed herein, or a pharmaceutically acceptable salt thereof. In some embodiments, the subject has a mutation in the SMARCAL1 gene. In some embodiments, the subject exhibits short stature, spondyloepiphyseal, intrauterine growth restriction (IUGR), nephropathy, bacterial infections, viral infections, fungal infections, SCID, bone marrow failure and/or low levels of T-cells. In some embodiments, the method corrects or treats short stature, spondyloepiphyseal, IUGR, nephropathy, bacterial infections, viral infections, fungal infections, SCID, bone marrow failure and/or low levels of T-cells.

In some embodiments, the present invention provides a method of treating MOPD1 deficiency, comprising administering to a subject in need thereof an effective amount of a CXCR4 inhibitor disclosed herein, or a pharmaceutically acceptable salt thereof. In some embodiments, the subject has a mutation in the RNU4ATAC gene. In some embodiments, the subject exhibits recurrent bacterial infections, lymphadenopathy, spondyloepiphyseal dysplasia, IUGR, retinal dystrophy, facial dysmorphism, microcephaly, short stature, low levels of Ig, and/or variably decreased specific antibodies. In some embodiments, the method corrects or treats recurrent bacterial infections, lymphadenopathy, spondyloepiphyseal dysplasia, IUGR, retinal dystrophy, facial dysmorphism, microcephaly, short stature, low Ig, and/or variably decreased specific antibodies.

In some embodiments, the present invention provides a method of treating MYSM1 deficiency, comprising administering to a subject in need thereof an effective amount of a CXCR4 inhibitor disclosed herein, or a pharmaceutically acceptable salt thereof. In some embodiments, the subject has a mutation in the MYSM1 gene. In some embodiments, the subject exhibits short stature, congenital bone marrow failure, myelodysplasia, skeletal anomalies, cataracts, developmental delay, affected granulocytes, immature B-cells, T-cell lymphopenia, reduced naïve T-cells, and/or hypogammaglobulinemia. In some embodiments, the method corrects or treats short stature, congenital bone marrow failure, myelodysplasia, skeletal anomalies, cataracts, developmental delay, affected granulocytes, immature B-cells, T-cell lymphopenia, reduced naïve T-cells, and/or hypogammaglobulinemia.

In some embodiments, the present invention provides a method of treating Facial dysmorphism, Immunodeficiency, Livedo, and Short stature (FILS) Syndrome, comprising administering to a subject in need thereof an effective amount of a CXCR4 inhibitor disclosed herein, or a pharmaceutically acceptable salt thereof. In some embodiments, the subject has a mutation in the POLE gene. In some embodiments, the subject exhibits low memory B-cells count, low naive T-cell count, and/or decreased T-cell proliferation. In some embodiments, the method corrects or treats low memory B-cells count, low naive T-cell count, and/or decreased T-cell proliferation.

In some embodiments, the present invention provides a method of treating Mannose-Binding Lectin (MBL) deficiency, comprising administering to a subject in need thereof an effective amount of a CXCR4 inhibitor disclosed herein, or a pharmaceutically acceptable salt thereof. In some embodiments, the subject has mutations in the MASP2, and/or FCN3 genes. In some embodiments, the subject exhibits increased risk of infection in toddlers, in cancer patients undergoing chemotherapy, and in organ-transplant patients receiving immunosuppressive drugs (particularly recipients of liver transplants), and/or high susceptibility, and severity of pneumonia in adults. In some embodiments, the method corrects or treats increased risk of infection in toddlers, in cancer patients undergoing chemotherapy, and in organ-transplant patients receiving immunosuppressive drugs (particularly recipients of liver transplants), and/or high susceptibility, and severity of pneumonia in adults.

In some embodiments, the present invention provides a method of treating Activated PI3K Delta Syndrome (ADPS), comprising administering to a subject in need thereof an effective amount of a CXCR4 inhibitor disclosed herein, or a pharmaceutically acceptable salt thereof. In some embodiments, the subject has mutations in the PIK3CD and/or PIK3R1 gene. In some embodiments, the subject exhibits frequent infections in the airways, frequent infections in the lungs, bronchiectasis, recurrent respiratory infections caused by Streptococcus pneumoniae and/or recurrent respiratory infections caused by Haemophilus influenzae. In some embodiments, the method corrects or treats frequent infections in the airways, frequent infections in the lungs, bronchiectasis, recurrent respiratory infections caused by Streptococcus pneumoniae and/or recurrent respiratory infections caused by Haemophilus influenzae.

In some embodiments, the present invention provides a method of treating X-linked Lymphoproliferative disease (XLP), comprising administering to a subject in need thereof an effective amount of a CXCR4 inhibitor disclosed herein, or a pharmaceutically acceptable salt thereof. In some embodiments, the subject has a mutation in the SH2D1A gene. In some embodiments, the subject exhibits recurrent infections, fulminant infectious mononucleosis and liver failure, and/or EBV or non-EBV induced hemophagocytic lymphohistiocytosis and lymphoma. In some embodiments, the method corrects or treats recurrent infections, fulminant infectious mononucleosis and liver failure, and/or EBV or non-EBV induced hemophagocytic lymphohistiocytosis and lymphoma.

In some embodiments, the present invention provides a method of treating X-linked Lymphoproliferative disease-2 (XLP-1), comprising administering to a subject in need thereof an effective amount of a CXCR4 inhibitor disclosed herein, or a pharmaceutically acceptable salt thereof. In some embodiments, the subject has a mutation in the XIAP gene. In some embodiments, the subject exhibits chronic Epstein-Barr virus (EBV) infection, splenomegaly, fever, colitis, inflammatory bowel disease (IBD), recurrent infections, hypogammaglobulinemia, cytopenias, low levels of NKT cells, and/or increased sensitivity of T cells to apoptosis. In some embodiments, the method corrects or treats chronic EBV infection, splenomegaly, fever, colitis, IBD, recurrent infections, hypogammaglobulinemia, cytopenias, low levels of NKT cells, and/or increased sensitivity of T cells to apoptosis.

In some embodiments, the present invention provides a method of treating XMEN disease, comprising administering to a subject in need thereof an effective amount of a CXCR4 inhibitor disclosed herein, or a pharmaceutically acceptable salt thereof. In some embodiments, the subject exhibits chronic EBV infection, EBV-related lymphoproliferative disease and/or low levels of CD4+ cells. In some embodiments, the method corrects or treats chronic EBV infection, EBV-related lymphoproliferative disease and/or low levels of CD4+ cells.

In some embodiments, the present invention provides a method of treating Chediak-Higashi syndrome (CHS), comprising administering to a subject in need thereof an effective amount of a CXCR4 inhibitor disclosed herein, or a pharmaceutically acceptable salt thereof. In some embodiments, the subject has mutations in the CYST and/or NLRC4 genes. In some embodiments, the subject exhibits Hemophagocytic lymphohistiocytosis (HLH), immune deficiency, increased susceptibility to infections, and/or a tendency to bruise and bleed easily. In some embodiments, the method corrects or treats HLH, immune deficiency, increased susceptibility to infections, and/or a tendency to bruise and bleed easily.

In some embodiments, the present invention provides a method of treating Griscelli syndrome (GS) Type 2, comprising administering to a subject in need thereof an effective amount of a CXCR4 inhibitor disclosed herein, or a pharmaceutically acceptable salt thereof. In some embodiments, the subject has a mutation in the RAB27A gene. In some embodiments, the subject exhibits immunologic abnormalities with or without neurologic impairment, leukocytes unable to stimulate normal lymphocytes, defect of helper T-cells, haemophagocytic syndrome, and/or uncontrolled T-lymphocyte and macrophage activation syndrome. In some embodiments, the method corrects or treats immunologic abnormalities with or without neurologic impairment, leukocytes unable to stimulate normal lymphocytes, defect of helper T-cells, haemophagocytic syndrome, and/or uncontrolled T-lymphocyte and macrophage activation syndrome.

In some embodiments, the present invention provides a method of treating Hermansky-Pudlak syndrome-2, comprising administering to a subject in need thereof an effective amount of a CXCR4 inhibitor disclosed herein, or a pharmaceutically acceptable salt thereof. In some embodiments, the subject has mutations in the AP3B1 and/or AP3D1 genes. In some embodiments, the subject exhibits increased susceptibility to infections due to congenital neutropenia, platelet defects and/or oculocutaneous albinism. In some embodiments, the method corrects or treats increased susceptibility to infections due to congenital neutropenia, platelet defects and/or oculocutaneous albinism.

In some embodiments, the present invention provides a method of treating Schwachman-Diamond Syndrome-Like (SDSL), comprising administering to a subject in need thereof an effective amount of a CXCR4 inhibitor disclosed herein, or a pharmaceutically acceptable salt thereof. In some embodiments, the subject has a mutation in the SRP54 gene. In some embodiments, the subject exhibits pancreatic dysfunction, neurologic abnormalities, severe congenital neutropenia-8 (SCN8), and/or recurrent bacterial infections (apparent from early infancy). In some embodiments, the method corrects or treats pancreatic dysfunction, neurologic abnormalities, severe congenital neutropenia-8 (SCN8), and/or recurrent bacterial infections (apparent from early infancy).

In some embodiments, the present invention provides a method of treating Roifman syndrome, comprising administering to a subject in need thereof an effective amount of a CXCR4 inhibitor disclosed herein, or a pharmaceutically acceptable salt thereof. In some embodiments, the subject exhibits intellectual disability, dysmorphic features, hypogonadism, humoral immunodeficiency, and/or recurrent sinopulmonary infections including otitis media and pneumonia. In some embodiments, the method corrects or treats intellectual disability, dysmorphic features, hypogonadism, humoral immunodeficiency, and/or recurrent sinopulmonary infections including otitis media and pneumonia.

In some embodiments, the present invention provides a method of treating Reticular dysgenesis, comprising administering to a subject in need thereof an effective amount of a CXCR4 inhibitor disclosed herein, or a pharmaceutically acceptable salt thereof. In some embodiments, the subject has a mutation in the AK2 gene. In some embodiments, the subject exhibits congenital agranulocytosis, lymphopenia, and/or lymphoid and thymic hypoplasia with absent cellular and humoral immunity functions. In some embodiments, the method corrects or treats congenital agranulocytosis, lymphopenia, and/or lymphoid and thymic hypoplasia with absent cellular and humoral immunity functions.

In some embodiments, the present invention provides a method of treating Immunoglobulin (IgG) subclass deficiency, comprising administering to a subject in need thereof an effective amount of a CXCR4 inhibitor disclosed herein, or a pharmaceutically acceptable salt thereof. In some embodiments, the subject exhibits problems with infections. In some embodiments, the method corrects or treats problems with infections.

In some embodiments, the present invention provides a method of treating selective IgM deficiency, comprising administering to a subject in need thereof an effective amount of a CXCR4 inhibitor disclosed herein, or a pharmaceutically acceptable salt thereof. IgM deficiency is also seen commonly in DOCK8 deficiency, typically in association with normal IgG and elevated IgE. In some embodiments, the subject exhibits recurrent infections and/or severe recurrent infections. In some embodiments, the method corrects or treats recurrent infections and/or severe recurrent infections.

In some embodiments, the present invention provides a method of treating Immunodeficiency, Centromeric region instability, Facial anomalies syndrome (ICF), comprising administering to a subject in need thereof an effective amount of a CXCR4 inhibitor disclosed herein, or a pharmaceutically acceptable salt thereof. In some embodiments, the subject has mutations in the DNMT3B and/or ZBTB24 gene. In some embodiments, the subject exhibits severe infections including pneumonia, sepsis, opportunistic infections caused by pathogens such as Candida albicans, Pneumocystis jiroveci, and JC virus, hypogammaglobulinemia and/or agammaglobulinemia. In some embodiments, the method corrects or treats severe infections including pneumonia, sepsis, opportunistic infections caused by pathogens such as Candida albicans, Pneumocystis jiroveci, and JC virus, hypogammaglobulinemia and/or agammaglobulinemia.

In some embodiments, the present invention provides a method of treating Charcot-Marie-Tooth Neutropenia, comprising administering to a subject in need thereof an effective amount of a CXCR4 inhibitor disclosed herein, or a pharmaceutically acceptable salt thereof. CMT-II is caused by mutation in the DNM2 gene. CMTDIB is caused by mutation in the DNM2 gene. CMTDIC caused by mutation of YARS gene. CMTDID caused by mutation of MPZ gene. CMTDIE caused by mutation of INF2 gene. CMTDIF caused by mutation of GNB4 gene. CMTDIG caused by mutation of NEFL gene. CMT2GG is caused by mutation of GBF1 gene. In some embodiments, the subject has mutations in the DNM2, YARS, MPZ, INF2, GNB4, NEFL, and/or GBF1 genes. In some embodiments, the subject exhibits CMT-II, CMTDIB, CMTDIC, CMTDID, CMTDIE, CMTDIF, CMTDIG, and/or CMT2GG. In some embodiments, the method corrects or treats CMT-II, CMTDIB, CMTDIC, CMTDID, CMTDIE, CMTDIF, CMTDIG, and/or CMT2GG.

In some embodiments, the present invention provides a method of treating X-Linked SCID, comprising administering to a subject in need thereof an effective amount of a CXCR4 inhibitor disclosed herein, or a pharmaceutically acceptable salt thereof. In some embodiments, the subject has mutations in the IL2RG and/or JAK3 genes.

In one aspect, the present invention provides a method of treating a secondary immune disorder selected from Phenytoin Toxicity and Fetal Hydantoin Syndrome, comprising administering to a subject in need thereof an effective amount of a CXCR4 inhibitor disclosed herein, or a pharmaceutically acceptable salt thereof. In some embodiments, the subject exhibits tumors and birth defects. In some embodiments, the method corrects or treats tumors and birth defects.

In one aspect, the present invention provides a method of treating an IL7 deficiency, comprising administering to a subject in need thereof an effective amount of a CXCR4 inhibitor disclosed herein, or a pharmaceutically acceptable salt thereof.

In one aspect, the present invention provides a method of treating a secondary immune disorder selected from At-Hook Transcription Factor (AKNA) and Preli Domain-Containing Protein 1 (PRELID1), comprising administering to a subject in need thereof an effective amount of a CXCR4 inhibitor disclosed herein, or a pharmaceutically acceptable salt thereof.

In another aspect, the present invention provides a method of treating a secondary immunodeficiency associated with malnutrition, metabolic disorders, inherited defects (e.g., Down Syndrome), surgery or trauma, chronic exposure to adverse environmental conditions, and infectious diseases such as measles, cytomegalovirus, or influenza, comprising administering to a subject in need thereof an effective amount of a CXCR4 inhibitor disclosed herein, or a pharmaceutically acceptable salt thereof.

### Common Variable Immune Deficiency (CVID)

Common variable immunodeficiency (CVID) is an antibody deficiency with an equal sex distribution and a high variability in clinical presentation. The main features include respiratory tract infections and their associated complications, enteropathy, autoimmunity, and lymphoproliferative disorders. CVIDs are estimated to occur with a prevalence of 1 per 25,000 people.

Abnormal B and T cell trafficking and altered CXCR4 expressions have been identified in CVID patients. Naive and memory B cell populations showed higher levels of expression of CXCR4 in CVID patients. Partial block in B-cell development at the pre-B-I to pre-B-II stage has been identified in CVID patients, correlating with lower transitional and mature B-cell counts in the periphery.

Several gene mutations have been found to be associated with CVID. Approximately 8% of affected individuals have TNFRSF13B (tumor necrosis factor receptor superfamily member 13B) gene mutations. The second most common gene associated with CVID is autosomal dominant gene, NFKB1 (Nuclear Factor Kappa B Subunit 1). Other dominant genes associated with CVID include NFKB2 (Nuclear Factor Kappa B Subunit 2), CLTA4, PI3KCD, IKZF1 and STAT3. Mutations in a recessive gene, LRBA, are also common in some groups. More rarely, mutations in CD19, CD81, ICOS CD20, CD21, and TNFRSF13C have been identified. Other genetic mutations possibly associated with CVID include, but are not limited to, activated p110δ syndrome (APDS), PIK3CD mutation (GOF; gain of function), PIK3R1 deficiency (LOF; loss of function), CD19 CD20 CD21 deficiencies, NFKB1 NFKB2 deficiencies IKAROS deficiency, CD81 deficiency, TNF-related weak inducer of apoptosis (TWEAK, also known as, Tumor necrosis factor ligand superfamily member 12; INJSF12) deficiency, Mannosyl-oligosaccharide glucosidase (MOGS) deficiency, TTC37 deficiency ATP6AP1 deficiency, BAFF Receptor deficiency, IRF2BP2 deficiency, Rho Guanine Nucleotide Exchange Factor 1 (ARHGEF1) deficiency, Protein transport protein Sec61 subunit alpha isoform 1 (SEC61A1), Ras-related C3 botulinum toxin substrate 2 (RAC2), tRNA-nucleotidyltransferase 1 (TRNT1), and TACI deficiency. See, e.g., Bonilla et al. (2016) J Allergy & ClinImmunol: InPractice 4:38-59, which is hereby incorporated by reference.

CVID leads to a variety of diseases and disorders, with a mortality of 15-30%. In one aspect, the present invention provides a method of treating CVID or diseases and disorders related to CVID, comprising administering to a patient in need thereof an effective amount of a CXCR4 inhibitor, such as mavorixafor, or a pharmaceutically acceptable salt or composition thereof. In one aspect, the present invention provides a method of treating an immune cell imbalance in a CVID patient, comprising administering to a patient in need thereof an effective amount of a CXCR4 inhibitor, such as mavorixafor, or a pharmaceutically acceptable salt or composition thereof.

In another aspect, the present invention provides a method of treating activated p110δ syndrome (APDS), comprising administering to a patient in need thereof an effective amount of a CXCR4 inhibitor, such as mavorixafor, or a pharmaceutically acceptable salt or composition thereof. In some embodiments, the patient shows elevated CXCR4 expression.

In another aspect, the present invention provides a method of treating CVID associated with a PIK3CD mutation, comprising administering to a patient in need thereof an effective amount of a CXCR4 inhibitor, such as mavorixafor, or a pharmaceutically acceptable salt or composition thereof.

In some embodiments, the CVID patient suffers from recurrent infections, polyclonal lymphoproliferation, autoimmune cytopenias, and/or granulomatous disease, with no gene defect specified. In one aspect, the present invention provides a method of treating recurrent infections, polyclonal lymphoproliferation, autoimmune cytopenias, and/or granulomatous disease, related to CVID, comprising administering to a patient in need thereof an effective amount of a CXCR4 inhibitor, such as mavorixafor, or a pharmaceutically acceptable salt or composition thereof.

In some embodiments, the method improves levels of the following to within 1.5 or 1 or 0.5 standard deviations from the mean: levels of IgG; levels of IgA and/or IgM; or all three. In some embodiments, the method provides an improved response to immunizations (protein and/or polysaccharide vaccines).

Most commonly, CVID patients suffer from recurrent infections, which is shown to be associated with, for example, CD19 deficiency, CD20 deficiency, CD21 deficiency, CD81 deficiency, TTC37 deficiency, and IRF2BP2 deficiency. In one aspect, the present invention provides a method of treating recurrent infections related to CVID, comprising administering to a patient in need thereof an effective amount of a CXCR4 inhibitor, such as mavorixafor, or a pharmaceutically acceptable salt or composition thereof, wherein the patient has one or more genetic mutations selected from CD19 deficiency, CD20 deficiency, CD21 deficiency, CD81 deficiency, TTC37 deficiency, and IRF2BP2 deficiency.

CVID patients suffer from severe bacterial infections, which have been shown to be associated with, for example, APDS, PIK3CD mutation (GOF), PIK3R1 deficiency (LOF), SH3KBP1 deficiency, reduced memory B-cells and increased transitional B-cells. In one aspect, the present invention provides a method of treating severe bacterial infections related to CVID, comprising administering to a patient in need thereof an effective amount of a CXCR4 inhibitor, such as mavorixafor, or a pharmaceutically acceptable salt or composition thereof, wherein the patient has one or more genetic mutations selected from APDS, PIK3CD mutation (GOF), PIK3R1 deficiency (LOF), and SH3KBP1 deficiency; or reduced memory B-cells and/or increased transitional B-cells.

In some embodiments, the patient has a deficiency in TACI or BAFF receptor. In some embodiments, the patient has altered TNFRSF13B (TACI) and/or TNFRSF13C (BAFF-R) expression.

In some embodiments, the CVID patient has a LOF mutation in PTEN gene. PTEN deficiency has been shown to cause lymphoproliferation, autoimmunity, and/or developmental delay in the patients carrying the mutation. In one aspect, the present invention provides a method of treating lymphoproliferation, autoimmunity, and/or developmental delay related to CVID, comprising administering to a patient in need thereof an effective amount of a CXCR4 inhibitor, such as mavorixafor, or a pharmaceutically acceptable salt or composition thereof, wherein the patient has PTEN deficiency.

CVID patients suffer from Epstein Barr virus (EBV), which has been shown to be associated with, for example, PIK3CD mutation (GOF) and PIK3R1 deficiency (LOF). In one aspect, the present invention provides a method of treating EBV related to CVID, comprising administering to a patient in need thereof an effective amount of a CXCR4 inhibitor, such as mavorixafor, or a pharmaceutically acceptable salt or composition thereof, wherein the patient has one or more genetic mutations comprising PIK3CD mutation (GOF) and PIK3R1 deficiency (LOF).

In some embodiments, the CVID patient has a LOF mutation in ARHGEF1 gene. ARHGEF1 deficiency has been shown to cause recurrent infections and/or bronchiectasis in the patients carrying the mutation. In one aspect, the present invention provides a method of treating recurrent infections and/or bronchiectasis related to CVID, comprising administering to a patient in need thereof an effective amount of a CXCR4 inhibitor, such as mavorixafor, or a pharmaceutically acceptable salt or composition thereof, wherein the patient has ARHGEF1 deficiency.

In some embodiments, the CVID patient has a LOF mutation in SEC61A1 gene. SEC61A1 deficiency has been shown to cause severe recurrent respiratory tract infections in the patients carrying the mutation. In one aspect, the present invention provides a method of treating severe recurrent respiratory tract infections related to CVID, comprising administering to a patient in need thereof an effective amount of a CXCR4 inhibitor, such as mavorixafor, or a pharmaceutically acceptable salt or composition thereof, wherein the patient has SEC61A1 deficiency.

In some embodiments, the CVID patient has a LOF mutation in RAC2 gene. RAC2 deficiency has been shown to cause recurrent sinopulmonary infections, poststreptococcal glomerulonephritis, urticaria, and/or IgA deficiency in the patients carrying the mutation. In one aspect, the present invention provides a method of treating recurrent sinopulmonary infections, poststreptococcal glomerulonephritis, urticaria, and/or IgA deficiency related to CVID, comprising administering to a patient in need thereof an effective amount of a CXCR4 inhibitor, such as mavorixafor, or a pharmaceutically acceptable salt or composition thereof, wherein the patient has RAC2 deficiency.

In some embodiments, the CVID patient has a LOF mutation in CD20 gene. CD20 deficiency has been shown to cause recurrent infections, low IgG and/or elevated IgM and IgA, in the patients carrying the mutation. In one aspect, the present invention provides a method of treating recurrent infections, related to CVID, comprising administering to a patient in need thereof an effective amount of a CXCR4 inhibitor, such as mavorixafor, or a pharmaceutically acceptable salt or composition thereof, wherein the patient has CD20 deficiency.

In some embodiments, the CVID patient has a LOF mutation in TWEAK, (also known as, TNFSF12) gene. TWEAK deficiency has been shown to cause pneumonia, bacterial infections, warts, thrombocytopenia, neutropenia, low IgM and IgA, and/or lack of anti-pneumococcal antibody, in the patients carrying the mutation. In one aspect, the present invention provides a method of treating pneumonia, bacterial infections, warts, thrombocytopenia, neutropenia, low IgM and IgA, and/or lack of anti-pneumococcal antibody, related to CVID, comprising administering to a patient in need thereof an effective amount of a CXCR4 inhibitor, such as mavorixafor, or a pharmaceutically acceptable salt or composition thereof, wherein the patient has TWEAK deficiency.

In some embodiments, the CVID patient has a LOF mutation in IRF2BP2 gene. IRF2BP2 deficiency has been shown to cause recurrent infections, autoimmunity and inflammatory disease, hypogammaglobinemia, and/or absent IgA, in the patients carrying the mutation. In one aspect, the present invention provides a method of treating recurrent infections, autoimmunity and inflammatory disease, hypogammaglobinemia, and/or absent IgA related to CVID, comprising administering to a patient in need thereof an effective amount of a CXCR4 inhibitor, such as mavorixafor, or a pharmaceutically acceptable salt or composition thereof, wherein the patient has IRF2BP2 deficiency.

In some embodiments, the CVID patient has a LOF mutation in CD19 gene. CD19 deficiency has been shown to cause recurrent infections, and/or glomerulonephritis in the patients carrying the mutation. In one aspect, the present invention provides a method of treating recurrent infections, and/or glomerulonephritis related to CVID, comprising administering to a patient in need thereof an effective amount of a CXCR4 inhibitor, such as mavorixafor, or a pharmaceutically acceptable salt or composition thereof, wherein the patient has CD19 deficiency

In some embodiments, the CVID patient has a LOF mutation in CD81 gene. CD81 deficiency has been shown to cause recurrent infections, and/or glomerulonephritis in the patients carrying the mutation. In one aspect, the present invention provides a method of treating recurrent infections, and/or glomerulonephritis related to CVID, comprising administering to a patient in need thereof an effective amount of a CXCR4 inhibitor, such as mavorixafor, or a pharmaceutically acceptable salt or composition thereof, wherein the patient has CD81 deficiency.

In some embodiments, the CVID patient has a LOF mutation in CD21 gene. CD21 deficiency has been shown to cause recurrent infections, low IgG, and/or impaired anti-pneumococcal response in the patients carrying the mutation. In one aspect, the present invention provides a method of treating recurrent infections, low IgG, and/or impaired anti-pneumococcal response, related to CVID, comprising administering to a patient in need thereof an effective amount of a CXCR4 inhibitor, such as mavorixafor, or a pharmaceutically acceptable salt or composition thereof, wherein the patient has CD21 deficiency.

In some embodiments, the CVID patient has a LOF mutation in TRNT1 gene. TRNT1 deficiency has been shown to cause congenital sideroblastic anemia, deafness, and/or developmental delay in the patients carrying the mutation. In one aspect, the present invention provides a method of treating congenital sideroblastic anemia, deafness, and/or developmental delay related to CVID, comprising administering to a patient in need thereof an effective amount of a CXCR4 inhibitor, such as mavorixafor, or a pharmaceutically acceptable salt or composition thereof, wherein the patient has TRNT1 deficiency.

In some embodiments, the CVID patient has a LOF mutation in NFKB1 gene. NFKB1 deficiency has been shown to cause recurrent sinopulmonary infections, COPD, and/or EBV proliferation in the patients carrying the mutation. In one aspect, the present invention provides a method of treating recurrent sinopulmonary infections, COPD, and/or EBV proliferation, related to CVID, comprising administering to a patient in need thereof an effective amount of a CXCR4 inhibitor, such as mavorixafor, or a pharmaceutically acceptable salt or composition thereof, wherein the patient has NFKB1 deficiency.

In some embodiments, the CVID patient has a LOF mutation in NFKB2 gene. NFKB2 deficiency has been shown to cause recurrent sinopulmonary infections, alopecia and/or endocrinopathies in the patients carrying the mutation. In one aspect, the present invention provides a method of treating recurrent sinopulmonary infections, alopecia and/or endocrinopathies related to CVID, comprising administering to a patient in need thereof an effective amount of a CXCR4 inhibitor, such as mavorixafor, or a pharmaceutically acceptable salt or composition thereof, wherein the patient has NFKB2 deficiency.

In some embodiments, the CVID patient has a LOF mutation in IKAROS gene. IKAROS deficiency has been shown to cause recurrent sinopulmonary infections in the patients carrying the mutation. In one aspect, the present invention provides a method of treating recurrent sinopulmonary infections related to CVID, comprising administering to a patient in need thereof an effective amount of a CXCR4 inhibitor, such as mavorixafor, or a pharmaceutically acceptable salt or composition thereof, wherein the patient has IKAROS deficiency.

In some embodiments, the CVID patient has a LOF mutation in ATP6AP1 gene. ATP6AP1 deficiency has been shown to hepatopathy, leukopenia, low copper leukopenia and/or hypogammagl in the patients carrying the mutation. In one aspect, the present invention provides a method of treating hepatopathy, leukopenia, low copper leukopenia and/or hypogammagl related to CVID, comprising administering to a patient in need thereof an effective amount of a CXCR4 inhibitor, such as mavorixafor, or a pharmaceutically acceptable salt or composition thereof, wherein the patient has ATP6AP1 deficiency.

In some embodiments, the CVID patient has a LOF mutation in MOGS gene. MOGS deficiency has been shown to cause severe neurologic disease in the patients carrying the mutation. In one aspect, the present invention provides a method of treating severe neurologic disease related to CVID, comprising administering to a patient in need thereof an effective amount of a CXCR4 inhibitor, such as mavorixafor, or a pharmaceutically acceptable salt or composition thereof, wherein the patient has MOGS deficiency.

In some embodiments, the CVID patient has a LOF mutation in TTC37 gene. TTC37 deficiency has been shown to cause trichorrhexis nodosa, and/or poor antibody response to pneumococcal vaccine in the patients carrying the mutation. In one aspect, the present invention provides a method of treating trichorrhexis nodosa, and/or poor antibody response to pneumococcal vaccine related to CVID, comprising administering to a patient in need thereof an effective amount of a CXCR4 inhibitor, such as mavorixafor, or a pharmaceutically acceptable salt or composition thereof, wherein the patient has TTC37 deficiency.

In one aspect, the present invention provides a method of treating diseases and disorders related to CVID, comprising administering to a patient in need thereof an effective amount of a CXCR4 inhibitor, such as mavorixafor, or a pharmaceutically acceptable salt or composition thereof.

### Congenital Defects Relating to Phagocytes

Various congenital defects of phagocyte number, function, or both are treatable by the methods provided by the present invention.

In some embodiments, the present invention provides a method of treating Shwachman-Diamond Sydrome, comprising administering to a subject in need thereof an effective amount of a CXCR4 inhibitor disclosed herein, or a pharmaceutically acceptable salt thereof. In some embodiments, the subject has a mutation in the DNAJC21 gene. In some embodiments, the subject has a mutation in the EFL1 gene. In some embodiments, the subject has a mutation in the SBDS gene. In some embodiments, the subject exhibits pancytopenia, and/or exocrine pancreatic insufficiency. In some embodiments, the method corrects or treats pancytopenia, and/or exocrine pancreatic insufficiency. In some embodiments, the subject exhibits chondrodysplasia. In some embodiments, the method corrects or treats chondrodysplasia in such a patient.

In some embodiments, the present invention provides a method of treating SRP54 deficiency, comprising administering to a subject in need thereof an effective amount of a CXCR4 inhibitor disclosed herein, or a pharmaceutically acceptable salt thereof. In some embodiments, the subject has a mutation in the SRP54 gene. In some embodiments, the subject exhibits neutropenia, and/or exocrine pancreatic insufficiency. In some embodiments, the method corrects or treats neutropenia, and/or exocrine pancreatic insufficiency.

In some embodiments, the present invention provides a method of treating G6PC3 deficiency (SCN4), comprising administering to a subject in need thereof an effective amount of a CXCR4 inhibitor disclosed herein, or a pharmaceutically acceptable salt thereof. In some embodiments, the subject has a mutation in the G6PC3 gene. In some embodiments, the subject exhibits structural heart defects, urogenital abnormalities, inner ear deafness, and/or venous angioectasias of trunks and limbs. In some embodiments, the method corrects or treats structural heart defects, urogenital abnormalities, inner ear deafness, and/or venous angioectasias of trunks and limbs. In some embodiments, in the subject certain functions, such as myeloid differentiation, chemotaxis, and/or O₂ production, are affected. In some embodiments, the method corrects or treats certain affected functions, such as myeloid differentiation, chemotaxis, and/or O₂ production.

In some embodiments, the subject does not have G6PC3 deficiency, and/or mutation in the G6PC3 gene, but the subject exhibits structural heart defects, urogenital abnormalities, inner ear deafness, and/or venous angioectasias of trunks and limbs. In some embodiments, the present invention provides a method, comprising administering to a subject in need thereof an effective amount of a CXCR4 inhibitor disclosed herein, or a pharmaceutically acceptable salt thereof, to correct or treat structural heart defects, urogenital abnormalities, inner ear deafness, and/or venous angioectasias of trunks and limbs, wherein the subject does not have G6PC3 deficiency, and/or mutation in the G6PC3 gene.

In some embodiments, the subject does not have G6PC3 deficiency, and/or mutation in the G6PC3 gene, but in the subject certain functions, such as myeloid differentiation, chemotaxis, and/or O₂ production, are affected. In some embodiments, the present invention provides a method, comprising administering to a subject in need thereof an effective amount of a CXCR4 inhibitor disclosed herein, or a pharmaceutically acceptable salt thereof, to correct or treat certain affected functions, such as, myeloid differentiation, chemotaxis, and/or O₂ production, wherein the subject does not have G6PC3 deficiency, and/or mutation in the G6PC3 gene.

In some embodiments, the patient does not exhibit idiopathic CD+ lymphocytopenia (ICL). In some embodiments, the patient does not have Wiskott-Aldrich Syndrome. In some embodiments, the patient does not have GATA2 deficiency.

In some embodiments, the present invention provides a method of treating glycogen storage disease type 1i, comprising administering to a subject in need thereof an effective amount of a CXCR4 inhibitor disclosed herein, or a pharmaceutically acceptable salt thereof. In some embodiments, the subject has a mutation in the G6PTI gene. In some embodiments, the subject exhibits fasting hypoglycemia, lactic acidosis, hyperlipidemia, and/or hepatomegaly. In some embodiments, the method corrects or treats fasting hypoglycemia, lactic acidosis, hyperlipidemia, and/or hepatomegaly.

In some embodiments, the present invention provides a method of treating Cohen syndrome, comprising administering to a subject in need thereof an effective amount of a CXCR4 inhibitor disclosed herein, or a pharmaceutically acceptable salt thereof. In some embodiments, the subject has a mutation in the COHJ gene. In some embodiments, the subject exhibits dysmorphism, mental retardation, obesity, and/or deafness. In some embodiments, the method corrects or treats dysmorphism, mental retardation, obesity, and/or deafness.

In some embodiments, the present invention provides a method of treating 3-Methylglutaconic aciduria, comprising administering to a subject in need thereof an effective amount of a CXCR4 inhibitor disclosed herein, or a pharmaceutically acceptable salt thereof. In some embodiments, the subject has a mutation in the CLPB gene. In some embodiments, the subject exhibits neurocognitive developmental aberrations, microcephaly, hypoglycemia, hypotonia, ataxia, seizures, cataracts, and/or intrauterine growth restriction (IUGR). In some embodiments, the method corrects or treats neurocognitive developmental aberrations, microcephaly, hypoglycemia, hypotonia, ataxia, seizures, cataracts, and/or IUGR.

In some embodiments, the present invention provides a method of treating Barth Syndrome (3-Methylglutaconic aciduria type II), comprising administering to a subject in need thereof an effective amount of a CXCR4 inhibitor disclosed herein, or a pharmaceutically acceptable salt thereof. In some embodiments, the subject has a mutation in the TAZ gene. In some embodiments, the subject exhibits cardiomyopathy, myopathy, and/or growth retardation. In some embodiments, the method corrects or treats cardiomyopathy, myopathy, and/or growth retardation.

In some embodiments, the present invention provides a method of treating Clericuzio syndrome (poikiloderma with neutropenia), comprising administering to a subject in need thereof an effective amount of a CXCR4 inhibitor disclosed herein, or a pharmaceutically acceptable salt thereof. In some embodiments, the subject has a mutation in the C160RF57 gene. In some embodiments, the subject exhibits retinopathy, developmental delay, facial dysmorphism, and/or poikiloderma. In some embodiments, the method corrects or treats retinopathy, developmental delay, facial dysmorphism, and/or poikiloderma.

In some embodiments, the present invention provides a method of treating VPS45 deficiency (SCN5), comprising administering to a subject in need thereof an effective amount of a CXCR4 inhibitor disclosed herein, or a pharmaceutically acceptable salt thereof. In some embodiments, the subject has a mutation in the VPS45 gene. In some embodiments, the subject exhibits extramedullary hematopoiesis, bone marrow fibrosis, and/or nephromegaly. In some embodiments, the method corrects or treats extramedullary hematopoiesis, bone marrow fibrosis, and/or nephromegaly.

In some embodiments, the present invention provides a method of treating JAGN1 (Jagunal Homolog 1) deficiency, comprising administering to a subject in need thereof an effective amount of a CXCR4 inhibitor disclosed herein, or a pharmaceutically acceptable salt thereof. In some embodiments, the subject has a mutation in the JAGN1 gene. In some embodiments, the subject exhibits osteopenia and/or myeloid maturation arrest. In some embodiments, the method corrects or treats osteopenia and/or myeloid maturation arrest.

In some embodiments, the present invention provides a method of treating WDR1 deficiency, comprising administering to a subject in need thereof an effective amount of a CXCR4 inhibitor disclosed herein, or a pharmaceutically acceptable salt thereof. In some embodiments, the subject has a mutation in the WDR1 gene. In some embodiments, the subject exhibits poor wound healing, severe stomatitis, neutrophil nuclei herniate, and/or mild neutropenia. In some embodiments, the method corrects or treats poor wound healing, severe stomatitis, neutrophil nuclei herniate, and/or mild neutropenia.

In some embodiments, the present invention provides a method of treating SMARCD2 deficiency, comprising administering to a subject in need thereof an effective amount of a CXCR4 inhibitor disclosed herein, or a pharmaceutically acceptable salt thereof. In some embodiments, the subject has a mutation in the SMARCD2 gene. In some embodiments, the subject exhibits developmental aberrations, bone defects, and/or myelodysplasia. In some embodiments, the method corrects or treats developmental aberrations, bone defects, and/or myelodysplasia.

In some embodiments, the present invention provides a method of treating specific granule deficiency, comprising administering to a subject in need thereof an effective amount of a CXCR4 inhibitor disclosed herein, or a pharmaceutically acceptable salt thereof. In some embodiments, the subject has a mutation in the CEBPE gene. In some embodiments, the subject exhibits neutrophils with bilobed nuclei and/or chronic neutropenia. In some embodiments, the method corrects or treats neutrophils with bilobed nuclei and/or chronic neutropenia.

In some embodiments, the present invention provides a method of treating HYOU1 deficiency, comprising administering to a subject in need thereof an effective amount of a CXCR4 inhibitor disclosed herein, or a pharmaceutically acceptable salt thereof. In some embodiments, the subject has a mutation in the HYOU1 gene. In some embodiments, the subject exhibits hypoglycemia, and/or inflammatory complications. In some embodiments, the method corrects or treats hypoglycemia, and/or inflammatory complications.

In some embodiments, the present invention provides a method of treating P14/LAMTOR2 deficiency, comprising administering to a subject in need thereof an effective amount of a CXCR4 inhibitor disclosed herein, or a pharmaceutically acceptable salt thereof. In some embodiments, the subject has a mutation in the LAMTOR2 gene. In some embodiments, the subject exhibits partial albinism, growth failure, hypogammaglobulinemia, and/or reduced CD8 cytotoxicity. In some embodiments, the method corrects or treats partial albinism, growth failure, hypogammaglobulinemia, and/or reduced CD8 cytotoxicity.

In some embodiments, the present invention provides a method of treating Elastase deficiency (SCN1), comprising administering to a subject in need thereof an effective amount of a CXCR4 inhibitor disclosed herein, or a pharmaceutically acceptable salt thereof. In some embodiments, the subject has a mutation in the ELANE gene. In some embodiments, the subject exhibits susceptibility to MDS/leukemia, severe congenital neutropenia, and/or cyclic neutropenia. In some embodiments, the method corrects or treats susceptibility to MDS/leukemia, severe congenital neutropenia, and/or cyclic neutropenia.

In some embodiments, the present invention provides a method of treating HAX1 deficiency (Kostmann Disease) (SCN3), comprising administering to a subject in need thereof an effective amount of a CXCR4 inhibitor disclosed herein, or a pharmaceutically acceptable salt thereof. In some embodiments, the subject has a mutation in the HAX1 gene. In some embodiments, the subject exhibits cognitive and neurological defects, and/or susceptibility to MDS/leukemia. In some embodiments, the method corrects or treats cognitive and neurological defects, and/or susceptibility to MDS/leukemia.

In some embodiments, the present invention provides a method of treating GFI 1 deficiency (SCN2), comprising administering to a subject in need thereof an effective amount of a CXCR4 inhibitor disclosed herein, or a pharmaceutically acceptable salt thereof. In some embodiments, the subject has a mutation in the GFI1 gene. In some embodiments, the subject exhibits B/T lymphopenia. In some embodiments, the method corrects or treats B/T lymphopenia.

In some embodiments, the present invention provides a method of treating X-linked neutropenia/myelodysplasia, comprising administering to a subject in need thereof an effective amount of a CXCR4 inhibitor disclosed herein, or a pharmaceutically acceptable salt thereof. In some embodiments, the subject has a GOF mutation in the WAS gene. In some embodiments, the subject exhibits myeloid maturation arrest, monocytopenia, and/or variable lymphoid anomalies. In some embodiments, the method corrects or treats myeloid maturation arrest, monocytopenia, and/or variable lymphoid anomalies.

In some embodiments, the subject does not have GOF mutation in the WAS gene, but the subject exhibits X-linked neutropenia/myelodysplasia. In some embodiments, the present invention provides a method, comprising administering to a subject in need thereof an effective amount of a CXCR4 inhibitor disclosed herein, or a pharmaceutically acceptable salt thereof, to correct or treat X-linked neutropenia/myelodysplasia, wherein the subject does not have GOF mutation in the WAS gene.

In some embodiments, the present invention provides a method of treating G-CSF receptor deficiency, comprising administering to a subject in need thereof an effective amount of a CXCR4 inhibitor disclosed herein, or a pharmaceutically acceptable salt thereof. In some embodiments, the subject has a mutation in the CSF3R gene. In some embodiments, the subject exhibits disturbed stress granulopoiesis. In some embodiments, the method corrects or treats disturbed stress granulopoiesis.

In some embodiments, the present invention provides a method of treating neutropenia with combined immune deficiency, comprising administering to a subject in need thereof an effective amount of a CXCR4 inhibitor disclosed herein, or a pharmaceutically acceptable salt thereof. In some embodiments, the subject has a mutation in the MKL1 gene. In some embodiments, the subject exhibits mild thrombocytopenia and/or lymphopenia. In some embodiments, the method corrects or treats mild thrombocytopenia and/or lymphopenia.

In some embodiments, treatment of particular sub-populations of patients with a CXCR4 inhibitor, or a pharmaceutically acceptable salt thereof, is particularly effective.

In some embodiments, the patient is male. In some embodiments, the patient is female.

In some embodiments, the patient is less than 50 years old. In some embodiments, the patient is at least 50 years old.

In some embodiments, the patient has previously been treated with one or more other immunomodulatory therapies.

In some embodiments, the CXCR4 inhibitor, and the other immunomodulatory therapies such as those described herein, act synergistically. Synergism includes, for example, more effective treatment of the disease than with either agent alone; or a lower dose of one or both agents providing effective treatment for the disease than would be the case if either agent were used alone.

In some embodiments, the patient has not previously been treated with any other immunomodulatory therapies prior to commencing treatment with CXCR4 inhibitor, or a pharmaceutically acceptable salt thereof.

In some embodiments, the patient is currently being treated with the immunomodulatory therapy. In some embodiments, the dose and/or frequency of administration of the other immunomodulatory therapy (while maintaining effectiveness of the treatment regimen) is/are reduced after treatment with CXCR4 inhibitor, or a pharmaceutically acceptable salt thereof, is commenced. In some embodiments, treatment with the other immunomodulatory therapy is completely discontinued (while maintaining effective treatment of the patient's immunodeficiency) after commencing treatment with CXCR4 inhibitor, or a pharmaceutically acceptable salt thereof.

In some embodiments, a provided method further comprises the step of obtaining a biological sample from the patient and measuring the amount of a disease-related biomarker. In some embodiments, the biological sample is a blood sample. In certain embodiments, the disease-related biomarker is selected from the group consisting of CXCR4, SDF-1α/CXCL12; and GRK3 (G protein coupled receptor kinase 3).

### Dosage and Formulations

The dose level and regimen may be set by the treating clinician, and typically depends on factors such as the age, weight, sex, and general health of the patient. In some embodiments, mavorixafor, or a pharmaceutically acceptable salt thereof, is administered in an oral dose, such as PO QD, of from about 25 mg/day to about 1200 mg/day. In some embodiments, the daily dose is from about 50 mg/day to about 800 mg/day; from about 100 mg/day to about 800 mg/day; from about 150 mg/day to about 800 mg/day; from about 200 mg/day to about 800 mg/day; from about 250 mg/day to about 800 mg/day; from about 300 mg/day to about 800 mg/day; from about 350 mg/day to about 800 mg/day; or from about 400 mg/day to about 800 mg/day.

In some embodiments, the daily dose is from about 100 mg/day to about 600 mg/day; from about 200 mg/day to about 600 mg/day; from about 300 mg/day to about 500 mg/day; or from about 350 mg/day to about 450 mg/day. In a particular embodiment, mavorixafor or a pharmaceutically acceptable salt thereof is administered in a daily dose of about 400 mg/day PO QD. Although the daily dose is preferably administered once daily, the clinician may also choose to divide the dose into two or more parts taken at intervals during the day. For example, a daily dose may be divided into two parts, with one half of the daily dose administered in the morning, and the second half of the daily dose administered in the afternoon or evening. The interval between halves of the daily dose may be from 4 hours to about 16 hours; preferably from about 5 hours to about 15 hours; or more preferably from about 6 hours to about 14 hours; from about 7 hours to about 13 hours; or from about 8 hours to about 12 hours.

In some embodiments, cells taken from the patient exhibit increased expression of CXCR4.

In some embodiments, the method further comprises the step of obtaining a biological sample from the patient and measuring the amount of a disease-related biomarker.

In some embodiments, the biological sample is a blood sample.

In some embodiments, the disease-related biomarker is ANC, ALC, total White Blood Cell counts (WBC), or circulating CXCR4.

In some embodiments, the mavorixafor or a pharmaceutically acceptable salt or composition thereof is administered orally (PO) once per day (QD).

In some embodiments, the mavorixafor or a pharmaceutically acceptable salt or composition thereof is administered orally (PO) twice per day (BID).

In some embodiments, a disclosed method comprises administering a mavorixafor unit dosage form comprising a composition comprising:
(a) mavorixafor, or a pharmaceutically acceptable salt thereof, as about 10-20% by weight of the composition;
(b) microcrystalline cellulose as about 70-85% by weight of the composition;
(c) croscarmellose sodium as about 5-10% by weight of the composition;
(d) sodium stearyl fumarate as about 0.5-2% by weight of the composition; and (e) colloidal silicon dioxide as about 0.1-1.0% by weight of the composition.

In some embodiments, the unit dosage form is in capsule form.

In some embodiments, the dosage form comprises about 25 mg mavorixafor, or a pharmaceutically acceptable salt thereof. In other embodiments, the dosage form comprises about 50 mg; 100 mg; 200 mg; 300 mg; 400 mg; 500 mg; 600 mg; or 800 mg mavorixafor, or a pharmaceutically acceptable salt thereof.

In some embodiments, the present invention provides a method for treating neutropenia, such as SCN or CIN, in a patient in need thereof, comprising the step of administering to the patient a disclosed unit dosage form.

In some embodiments, the present invention provides a method for treating neutropenia, such as SCN or CIN, in a patient in need thereof, comprising administering to said patient mavorixafor, or a pharmaceutically acceptable salt or composition thereof, in an amount effective to increase absolute neutrophil count (ANC) and/or to increase absolute lymphocyte count (ALC) in the patient, for example in the patient's blood. In some embodiments, the ANC and/or ALC is increased in the patient by at least 10%, 15%, 20%, 25%, 30%, 35%, 40%, 45% or at least 50% of that of the pre-treatment baseline counts.

In some embodiments, the present invention provides a method for treating neutropenia, such as SCN or CIN, in a patient in need thereof, comprising administering to said patient mavorixafor or a pharmaceutically acceptable salt or composition thereof, in an amount effective to increase absolute neutrophil count (ANC) to a level greater than or equal to 500/μL and/or to increase absolute lymphocyte count (ALC) to a level greater than or equal to 1000/μL.

In some embodiments, said patient originally exhibits ANC less than 600/μL and/or ALC less than 1000/μL before treatment with mavorixafor, or a pharmaceutically acceptable salt or composition thereof.

In some embodiments, said patient originally exhibits ANC less than 500/μL and/or ALC less than 650/μL before treatment with mavorixafor or a pharmaceutically acceptable salt or composition thereof.

In some embodiments, a disclosed method results in increases in ANC levels to at least about 500/μL, at least about 600/μL, at least about 700/μL, at least about 800/μL, at least about 900/μL, at least about 1000/μL, at least about 1,100/μL, or at least about 1,200/μL, or to about that of a human with a normally-functioning immune system, on at least 85% of assessments.

In some embodiments, a disclosed method results in increases in ALC to at least about 1000/μL, about 1,200/μL, or about 1,500/μL, or to about that of a human with a normally-functioning immune system, on at least 85% of assessments.

In some embodiments, a disclosed method results in a lowered frequency of infections in the patient, such as at least 10%; at least 25%; or at least 50% less infections. In some embodiments, the method reduces the frequency of a respiratory tract infection.

In some embodiments, a disclosed method results in increased levels of total circulating WBC, neutrophils, and/or lymphocytes. In some embodiments, cell counts of WBC, neutrophils, and/or lymphocytes increase to approximately 1.4×baseline. In some embodiments, cell counts of WBC, neutrophils, and/or lymphocytes increase to approximately 1.6×baseline, 1.8×baseline, or 2.0×baseline. In some embodiments, cell counts of WBC, neutrophils, and/or lymphocytes increase to approximately 2.9×baseline. In some embodiments, cell counts of lymphocytes increase to approximately 2.9×baseline. In some embodiments, cell counts of neutrophils increase to approximately 2.7×baseline and lymphocytes to approximately 1.9×baseline.

In some embodiments, the present invention provides a method of treating neutropenia, such as SCN or CIN, in a patient in need thereof, wherein said method comprises administering to said patient an effective amount of mavorixafor or a pharmaceutically acceptable salt or composition thereof in conjunction with another treatment for neutropenia, such as SCN or CIN.

In some embodiments, the present invention provides a method of treating neutropenia, such as SCN or CIN, in a patient in need thereof, wherein said patient has been either receiving no treatment or receiving regular or preventative treatment with G-CSF, or a variant thereof. The method comprises administering to said patient an effective amount of mavorixafor. The timing of administration of mavorixafor may be prior to, together with, or subsequent to administration of G-CSF, or a variant thereof.

In certain embodiments, after commencement of administration of mavorixafor, the dosage of G-CSF administered to said patient may be reduced, while maintaining absolute neutrophil counts (ANC) equal to or higher than 500 cells/L.

In certain embodiments, the dosage of G-CSF that is administered to the patient is reduced by at least about 25% relative to the patient's previous dose before beginning treatment with mavorixafor or a pharmaceutically acceptable salt or composition thereof. In certain embodiments, the dosage of G-CSF that is administered to the patient is reduced by at least about 50%, 75%, or 95% relative to the patient's previous dose before beginning treatment with mavorixafor or a pharmaceutically acceptable salt or composition thereof. In certain embodiments, the dosage of G-CSF or GM-CSF, or variant thereof, that is administered to the patient is reduced by about 10%, 15%, 20%, 25%, 30%, 35%, 40%, 50%, 55%, 60, 65, 70%, 75%, 80%, 85%, 90%, or 95%.

In certain embodiments, the frequency of dosage of G-CSF or GM-CSF or variant thereof is reduced, for example, reduced in frequency by at least 25%, 50%, 75%, or 90%.

In certain embodiments, administration of G-CSF or GM-CSF, or variant thereof, may be eliminated, or administered only in the event of a crisis, for example, if ANC levels drop below 500 cells/μL. Decreased dosage of G-CSF or GM-CSF, or variant thereof, can be effected by lowering the doses administered at any one time and/or by increasing the interval between dosage administration, e.g., once every three days, rather than once every two days.

In some embodiments, the patient begins with a well-tolerated dose of oral, daily mavorixafor, for example, 400 mg per day, wherein the patient is presently receiving a full dose (1X) of G-CSF or peg-G-CSF. The patient is typically monitored for ANC. In some embodiments, if the patient's ANC is at or above 1000 cells/μL, patient's dose of G-CSF or peg-G-CSF is reduced by a factor of approximately 25%, (i.e., to 0.75× dose). In some embodiments, if ANC remains at or above 1000 cells/μL, then (a) the patient's dose of G-CSF or peg-G-CSF is further reduced; (b) the daily dosage of mavorixafor being administered is increased or decreased; or both (a) and (b). Typically, at such time, ANC will continue to be monitored, with a goal of ANC of at least 500 cell/μL being maintained. As long as the patient's ANC remains above 500 cells/μL, the patient's dose of G-CSF or peg-G-CSF is optionally further reduced. In some embodiments, the method reduces bone pain or other adverse effects of G-CSF or peg-G-CSF.

If the patient's measured ANC is found to be between 500 and 1000 cells/μL, (a) the patient's dose of G-CSF or peg-G-CSF is further reduced; (b) the daily mavorixafor dosage is increased; or both (a) and (b). In some embodiments, the method provides maintenance of an ANC of at least 500 cell/μL. In some embodiments, as long as the patient's ANC remains above 500 cells/μL, the patient's dose of G-CSF or peg-G-CSF is optionally further reduced. In some embodiments, the method reduces bone pain or other adverse effects of G-CSF or peg-G-CSF.

### Mavorixafor

CXCR4 inhibitors such as the compound mavorixafor (previously known as X4P-001, AMD070, or AMD11070) or a pharmaceutically acceptable salt thereof or pharmaceutical composition thereof, as described in greater detail below, are useful both as a monotherapy and as a combination therapy with one or more other therapeutic agents described herein. Accordingly, in one aspect, the present invention provides a method of treating neutropenia, such as those described herein, by administering to a patient in need thereof an effective amount of a CXCR4 inhibitor such as mavorixafor, or a pharmaceutically acceptable salt thereof or pharmaceutical composition thereof. In some embodiments, the method further includes co-administering simultaneously or sequentially an effective amount of one or more additional therapeutic agents, such as those described herein.

Mavorixafor (formerly known as X4P-001, AMD 070, or AMD 11070) is a small molecule antagonist of CXCR4 having the potential to block the enhanced signaling activity of wild type and mutant CXCR4, resulting in an increase in the number of circulating white blood cells (Leukocytosis) of 2.9-fold (400-mg single-dose subject) above baseline with a peak between 2 and 4 h following dosing (Stone, 2007) by inhibiting CXCR4-dependent interactions between bone marrow stromal cells and mature leukocytes of many lineages thus allowing release of these cells into the circulation (Liles Blood 2003).

Mavorixafor is a second-generation, small-molecule, non-competitive, allosteric antagonist of chemokine receptor type 4 (CXCR4) that acts by binding to extracellular domains of the receptor, resulting in specific and reversible inhibition of receptor signaling in response to its ligand C-X-C motif chemokine ligand 12 (CXCL12). Mavorixafor is currently in clinical development in patients with cancer (renal cell carcinoma), Waldenström Macroglobulinemia, and with warts, hypogammaglobulinemia, infections, and myelokathexis (WHIM) syndrome. The chemical formula is: C₂₁H₂₇N₅; and molecular weight is 349.48 amu. The chemical structure of mavorixafor is as follows according to Formula I:

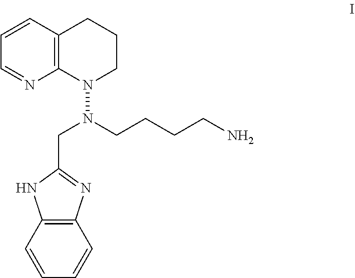

As of May 2019, approximately 193 healthy volunteers and patients had been treated with mavorixafor in clinical studies (n=70 healthy volunteers, n=16 HIV, n=99 oncology, n=8 WHIM syndrome). Overall, mavorixafor has been generally well tolerated, with no mavorixafor-related serious AEs (SAEs) causing a fatal outcome in any of the patients.

In certain embodiments, the mavorixafor, pharmaceutically acceptable salt thereof, or composition comprising mavorixafor or a pharmaceutically acceptable salt thereof is administered orally (PO) once daily (QD) or twice daily (BID), in an amount from about 25 mg to about 800 mg daily. In certain embodiments, the dosage composition may be provided twice a day in divided dosage, approximately 12 hours apart. In other embodiments, the dosage composition may be provided once daily. The terminal half-life of mavorixafor has been generally determined to be between about 12 to about 24 hours, or approximately 14.5 hrs. In certain embodiments, the dosage of mavorixafor useful in the invention is from about 25 mg to about 1200 mg daily. In other embodiments, the dosage of mavorixafor useful in the invention may range from about 25 mg to about 1000 mg daily, from about 50 mg to about 800 mg daily, from about 50 mg to about 600 mg daily, from about 50 mg to about 500 mg daily, from about 50 mg to about 400 mg daily, from about 100 mg to about 800 mg daily, from about 100 mg to about 600 mg daily, from about 100 mg to about 500 mg daily, from about 100 mg to about 400 mg daily; from about 200 mg to about 800 mg daily, from about 200 mg to about 600 mg daily, from about 300 mg to about 600 mg daily, from about 200 mg to about 500 mg daily from about 200 mg to about 400 mg daily.

In other embodiments, the dosage of mavorixafor or a pharmaceutically acceptable salt thereof is administered in a dosage range from about 100 mg to about 800 mg daily, from about 200 mg to about 600 mg daily, from about 300 mg to about 500 mg daily, or from about 350 mg to about 450 mg daily; or in a daily dosage of about 100 mg/day; 125 mg/day; 150 mg/day; 175 mg/day; 200 mg/day; 225 mg/day; 250 mg/day; 275 mg/day; 300 mg/day; 325 mg/day; 350 mg/day; 400 mg/day; 425 mg/day; 450 mg/day; 475 mg/day; 500 mg/day; 525 mg/day; 550 mg/day; 575 mg/day; 600 mg/day; 625 mg/day; 650 mg/day; 675 mg/day; 700 mg/day; 725 mg/day; 750 mg/day; 775 mg/day or 800 mg/day. In unusual cases, the dosage of mavorixafor or a pharmaceutically acceptable salt thereof may be administered in an amount in excess of 800 mg/day, while taking care to minimize or avoid any adverse effects of such administration.

In some embodiments, a provided method comprises administering to the patient a pharmaceutically acceptable composition comprising mavorixafor wherein the composition is formulated for oral administration. In certain embodiments, the composition is formulated for oral administration in the form of a tablet, a caplet or a capsule. In some embodiments, the composition comprising mavorixafor is formulated for oral administration in the form of a capsule.

In certain embodiments, a provided method comprises administering to the patient one or more dosage forms comprising 25 mg to 1200 mg mavorixafor active ingredient; and one or more pharmaceutically acceptable excipients. In certain embodiments, the capsule is comprised of hard gelatin. In some embodiments the dosage form comprises 25 mg to 800 mg mavorixafor active ingredient, 50 mg to 600 mg mavorixafor active ingredient, 100 mg to 500 mg mavorixafor active ingredient, 100 mg to 400 mg mavorixafor active ingredient, 100 mg to 300 mg mavorixafor active ingredient, or 100 mg to 200 mg mavorixafor active ingredient.

In certain embodiments, a disclosed method comprises administering a composition comprising mavorixafor, or a pharmaceutically acceptable salt thereof, one or more diluents, a disintegrant, a lubricant, a flow aid, and a wetting agent. In some embodiments, a disclosed method comprises administering a composition comprising 25 mg to 1200 mg mavorixafor, or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In some embodiments, a disclosed method comprises administering a unit dosage form wherein said unit dosage form comprises a composition comprising 25 mg to 200 mg mavorixafor, or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In certain embodiments, a disclosed method comprises administering a unit dosage form comprising a composition comprising mavorixafor, or a pharmaceutically acceptable salt thereof, present in an amount of about 25 mg, about 40 mg, about 50 mg, about 80 mg, about 100 mg, about 150 mg, about 200 mg, about 250 mg, about 300 mg, about 350, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg. In some embodiments, a provided composition (or unit dosage form) is administered to the patient once per day, twice per day, three times per day, or four times per day. In some embodiments, a provided composition (or unit dosage form) is administered to the patient once per day or twice per day.

In some embodiments, a disclosed method comprises administering a unit dosage form comprising a composition comprising:
(a) mavorixafor, or a pharmaceutically acceptable salt thereof, as about 10-30% by weight of the composition;
(b) microcrystalline cellulose as about 60-80% by weight of the composition;
(c) croscarmellose sodium as about 5-10% by weight of the composition;
(d) sodium stearyl fumarate as about 0.5-2% by weight of the composition; and
(e) colloidal silicon dioxide as about 0.1-1.0% by weight of the composition.

In some embodiments, a disclosed method comprises administering a unit dosage form comprising a composition comprising:
(a) mavorixafor, or a pharmaceutically acceptable salt thereof, as about 15% by weight of the composition;
(b) microcrystalline cellulose as about 78% by weight of the composition;
(c) croscarmellose sodium as about 6% by weight of the composition;
(d) sodium stearyl fumarate as about 1% by weight of the composition; and
(e) colloidal silicon dioxide as about 0.2% by weight of the composition.

In some embodiments, a disclosed method comprises administering a unit dosage form comprising a composition comprising:
(a) mavorixafor, or a pharmaceutically acceptable salt thereof, as about 10-20% by weight of the composition;
(b) microcrystalline cellulose as about 25-40% by weight of the composition;
(c) dibasic calcium phosphate dihydrate as about 35-55% by weight of the composition;
(d) croscarmellose sodium as about 4-15% by weight of the composition;
(e) sodium stearyl fumarate as about 0.3-2% by weight of the composition;
(f) colloidal silicon dioxide as about 0.1-1.5% by weight of the composition; and
(g) sodium lauryl sulfate as about 0.1-1.5% by weight of the composition.

In some embodiments, a disclosed method comprises administering a unit dosage form comprising a composition comprising:
(a) mavorixafor, or a pharmaceutically acceptable salt thereof, as about 13% by weight of the composition;
(b) microcrystalline cellulose as about 32% by weight of the composition;
(c) dibasic calcium phosphate dihydrate as about 44% by weight of the composition;
(d) croscarmellose sodium as about 8% by weight of the composition;
(e) sodium stearyl fumarate as about 1.4% by weight of the composition;
(f) colloidal silicon dioxide as about 0.4% by weight of the composition; and
(g) sodium lauryl sulfate as about 0.7% by weight of the composition.

In some embodiments, a disclosed method comprises administering a unit dosage form comprising a composition comprising:
(a) mavorixafor, or a pharmaceutically acceptable salt thereof, as about 35-75% by weight of the composition;
(b) microcrystalline cellulose as about 5-28% by weight of the composition;
(c) dibasic calcium phosphate dihydrate as about 7-30% by weight of the composition;
(d) croscarmellose sodium as about 2-10% by weight of the composition;
(e) sodium stearyl fumarate as about 0.3-2.5% by weight of the composition;
(f) colloidal silicon dioxide as about 0.05-1.2% by weight of the composition; and
(g) sodium lauryl sulfate as about 0.2-1.2% by weight of the composition.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions. Thus, the kit of the invention includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

The contents of each document cited in the specification are herein incorporated by reference in their entireties.

### Exemplification

### Example 1. CXCR4 Receptor Expression

Rationale: This assay measures the levels of CXCR4 receptor on the cell surface of cells of interest. Upregulation of the receptor can make the cells hyperresponsive to CXCL12.

Procedure: Cells (cell lines, primary patient cells) are seeded in 96-well plates. Cells are then resuspended in incubation buffer (Hanks' Balanced Salt Solution [HBSS] with Ca²⁺ and Mg²⁺+0.5% BSA+20 mM HEPES buffer pH 7.4) then stained with anti-CXCR4 12G5-APC monoclonal antibody (BD Biosciences; 1:20 dilution in incubation buffer) for 20 min. After washing and resuspending in flow buffer (HBSS with Ca²⁺ and Mg²⁺+0.1% BSA+20 mM HEPES buffer pH 7.4), the samples are measured via flow cytometry (Cytoflex) and analyzed using flow cytometry software (FCS Express). Cells are gated based on the forward and side scatter (FSC and SSC, respectively) and the mean fluorescent intensity (MFI) of the population is determined and compared to isotype-stained cells.

### Example 2. CXCR4 Receptor Internalization

### Rationale:

This assay measures decrease of surface CXCR4 as a response to CXCL12 stimulation. Impairment of this process leads to hyperactive receptor.

### Procedure:

Cell expressing CXCR4 (cell lines, primary patient cells) are seeded in 96-well plates. Cells are then resuspended in warm incubation buffer (Hanks' Balanced Salt Solution [HBSS] with Ca²⁺ and Mg²⁺+0.5% BSA+20 mM HEPES buffer pH 7.4) and stimulated with CXCL12 for 45 min or 4 h at 37° C., 5% CO₂. After incubation, the cells are washed twice with cold incubation buffer and then stained with anti-CXCR4 12G5-APC monoclonal antibody (BD Biosciences; 1:20 dilution in incubation buffer) for 20 min at 4° C. After washing and resuspending in flow buffer (HBSS with Ca²⁺ and Mg²⁺+0.1% BSA+20 mM HEPES buffer pH 7.4), the samples are measured via flow cytometry (Cytoflex) and analyzed using flow cytometry software (FCS Express). Cells are gated based on the forward and side scatter (FSC and SSC, respectively) and isotype control, and the mean fluorescent intensity (MFI) of the CXCR4+ population is used in subsequent analysis. % surface CXCR4 expression is calculated according to formula: MFI/MFI_{NC}*100, where MFI stands for mean fluorescent intensity of sample treated by ligand and MFI_{NC} is mean fluorescent intensity of sample treated by vehicle.

### Example 3. Chemotaxis Assay

Rationale: This assay measures migration capacity of cells toward CXCL12 ligand.

Cells with CXCR4 Gain-of-Function Display Enhanced Chemotaxis.

Procedure: Prior to chemotaxis, cells (cell lines, primary patient cells) are stained with 500 nM Calcein AM Viability Dye (Thermo Fisher Scientific, Waltham, Massachusetts, USA) in medium for 15 min at room temperature in the dark. Aliquots of cells in medium are added to the plate inserts in transwell plates (Corning Incorporated, Corning, New York, USA) and 600 μl buffer with increasing CXCL12 concentrations is added to the bottom wells. Cells are allowed to migrate in response to CXCL12 at 37° C. and 5% CO₂ for 2-4 hours. After removal of the plate inserts, the migrated cells are centrifuged and resuspended in Dulbecco's phosphate buffered saline containing flow cytometry counting beads (Precision Count Beads™ BioLegend, San Diego, California, USA). Both migrated cells and counting beads are counted by flow cytometry (Cytoflex). Data are analyzed using flow cytometry software (FCS Express). The total number of migrated cells is calculated according to the counted and total number of beads present in the sample.

### Example 4. Adhesion Assay

Rationale: This assay measures the ability of cells to adhere to bone marrow stroma cells (BMSCs). Cells with CXCR4 gain-of-function may display enhanced adhesion.

Procedure: HS27a BMSCs (ATCC) are seeded at a density of 5×10⁴ cells/well in complete medium (RPMI medium supplemented with 1% Penicillin-Streptomycin (both from Gibco) and 10% FBS (Sigma) in a 24-well plate and allowed to form a monolayer over 48 to 72 hours. On the day of adhesion assay, cells (cell lines or primary patient cells) are washed twice with DPBS (Gibco) and stained with 1 μM Calcein AM (Thermo Fisher Scientific) for 15 minutes at 37° C. Afterwards, cells are washed twice with DPBS and pre-incubated with tested compounds (e.g. CXCR4 antagonists) for 30 minutes at 37° C. The labelled and treated cells are then plated onto the pre-established monolayer of HS27a cells and allowed to adhere for 3 hours at 37° C. Following 3 hours co-incubation, the non-adherent cells are washed from the wells, and adherent cell fraction (including BMSCs and cells of interest) is harvested with trypsin/EDTA (Gibco) and measured by flow cytometry (CytoFLEX Flow Cytometer) and analyzed in FCS Express software. Calcein AM-unlabeled BMSCs are gated out.

### Example 5. Neutrophil Stimulation

Neutrophils (1.25×10⁶ cells/mL) are pre-treated 15 minutes with BTKi at 37° C. and then stimulated with zymosan (50 g/ml), heat-inactivated C. albicans (1:15) or A. fumigatus (1:150) for 1 h. Cells are then incubated with anti-CD11b and CD62L and neutrophil activation is evaluated by flow cytometry.

To assess the production of reactive oxygen species (ROS) neutrophils are stained for 15 minutes at 37° C. with dihydrorhodamine 123 (DHR), treated for 15 minutes with BTKi (1.25×10⁶ cells/mL) and then incubated for 30 minutes with zymosan (500 g/ml) or heat-inactivated C. albicans (1:65) or for 1 h with A. fumigatus (1:150). Rhodamine fluorescence is then evaluated by flow cytometry.

For neutrophil phagocytosis, cells (1.25×10⁶ cells/mL) were incubated with BTKi for 15 minutes, cultured with zymosan-, C. albicans-, A. fumigatus—FITC for 1.5 h and then phagocytosis evaluated by flow cytometry and confirmed by confocal microscopy.

### Example 6. Macrophages Cultures

Macrophages were obtained from HD- or CLL patient-monocytes by culturing them (0.75×10⁶ cells/mL) for five days in RPMI 1640 10% FCS in the presence of M-CSF (50 ng/ml). For the phagocytosis assay, macrophages were pre-treated with BTKi for 30 minutes and then cultured for 2 h with zymosan-, heat inactivated C. albicans yeast- or heat inactivated A. fumigatus conidia—FITC and evaluated by flow cytometry. To evaluate the effect of BTKi on cytokine secretion in response to fungal stimulation, macrophages are pre-treated with BTKi, stimulated with zymosan (10 g/ml), heat-inactivated C. albicans (1:20) or heat-inactivated A. fumigatus conidia (1:1) and after 24 h TNF-α secretion is measured in culture supernatants by ELISA.

To obtain M1 macrophage, monocytes (0.75×10⁶ cells/mL) are cultured in RPMI 1640 medium with 10% FCS for five days with GM-CSF (50 ng/ml) and two additional days with GM-CSF+IFN-7 (10 ng/ml) [Colado et al. (2018) Blood Cancer Journal. 8:100 “Effect of the BTK inhibitor ibrutinib on macrophage- and gamma-delta T cell-mediated response against Mycobacterium tuberculosis” ]. To evaluate the expression of the markers by flow cytometry, at day 7, cells are detached with cold PBS 2% FCS 2 mM EDTA and stained with anti-CD14, anti-CD16, anti-CD163, anti-CD206, anti-CD86 and anti-HLA-DR. To assesses the spontaneous secretion of cytokines in culture supernatants, after polarization, culture medium is replaced by fresh medium and after 24 h TNF-α and IL-10 are measured by ELISA.

### Example 7. Neutrophils Mediated Inhibition of Germination of Aspergillus fumigatus Conidia

Following a previous published protocol [See Gazendam et al. (2016) Journal of immunology. 196:1272-83 “Human Neutrophils Use Different Mechanisms To Kill Aspergillus fumigatus Conidia and Hyphae: Evidence from Phagocyte Defects” ], A. fumigatus conidia (6×10⁶ conidia/mL) are incubated in a 96 well plate for 3 h in RPMI 1640 10% FCS. Neutrophils (1.25×10⁶ cells/mL) are treated with 1 μM of BTKi for 15 minutes and then added to A. fumigatus conidia cultures for 6 h. After that, half of the culture medium is replaced by SDS 0.5% for 15 min at room temperature, to lyse the neutrophils. Then, the medium is replaced by fresh medium containing MTT (0.5 mg/ml). Culture plates are incubated at 37° C. and after addition of DMSO, the OD is measured on a plate reader at 570 nm. The percentage of A. fumigatus conidia germination is calculated as the OD vs. control OD (culture without neutrophils). In order to obtain microscopy images, after the 6 h of culture, medium is replaced by PFA 4% for 30 minutes at room temperature.

### Example 8. ADCP, ADCC and Degranulation

For the antibody-dependent phagocytosis (ADCP) assay, CFSE (1 μM) labelled CLL cells are coated or not for 30 minutes [with rituximab (Rx) or obinutuzumab (Obz)](50 g/ml). Macrophages (0.75×10⁶ cells/mL) are pre-treated for 30 minutes with BTKi and then cultured with the opsonized CLL cells (1:4). After 1 hour macrophages are detached with trypsin and phagocytosis is evaluated by flow cytometry as the percentage of macrophages FITC⁺ [Da Roit et al. (2015) Haematologica. 100:77-86; “Ibrutinib interferes with the cell-mediated anti-tumor activities of therapeutic CD20 antibodies: implications for combination therapy”; Borge et al. (2015) Haematologica; 100:e140-2 “Ibrutinib impairs the phagocytosis of rituximab-coated leukemic cells from chronic lymphocytic leukemia patients by human macrophages” ]. For antibody-dependent cytotoxicity (ADCC) assay DAUDI cell line (ATCC CCL-213) is used as target, because this cell line express higher levels of CD20 than CLL cells and therefore are more efficiently killed by NK cells. Daudi cells are labelled with CFSE, coated or not with Rx or Obz for 30 minutes and then cultured with PBMC (15:1) that are previously pre-treated 30 minutes with BTKi. After 4 hours, cells are stained with 7ADD and evaluated by flow cytometry to determine the percentage of cells CFSE⁺7AAD⁺. For NK degranulation assay, CFSE⁺ CLL cells coated or not with Rx or Obz are cultured with PBMC, that are previously pre-treated 30 minutes with BTKi (1:1), in the presence of PE-conjugated anti-CD107a mAb and monensin (2 M). After 4 h, cells are stained with anti-CD56 and evaluated by flow cytometry. See: Colado et al. (2020) Am J Hematol 95:E174-E178 (Supplementary Materials).

### Example 9. Measuring Effectiveness of CXCR4 Inhibitors; Activation of the AKT/ERK Pathway

Rationale: This assay measures the activity of compounds to inhibit ERK and AKT pathways activated downstream of CXCR4 receptor.

Procedure: Cells (cell lines or primary patient cells) are seeded in starvation medium in 96-well. Cells were pretreated by serial dilutions of compounds for 15 min. Subsequently the cells are treated with CXCL12 (10 nM, Peprotech) for desired time (5 min, 30 min). After treatment the cells are fixed with fixation solution (eBioscience, Thermo Fisher Scientific, Waltham, Massachusetts, USA) for 10 min at room temperature, then permeabilized for 5 min with ice-cold methanol on ice followed by a washing step with wash/permeabilization solution (eBioscience, Thermo Fisher Scientific, Waltham, Massachusetts, USA). Cells are then stained with Alexa Fluor 647 Mouse Anti-ERK1/2 (pT202/pY204, 1/10 dilution, BD Biosciences) and Alexa Fluor 488 Mouse anti-Akt (pS473, 1/10 dilution, BD Biosciences) for 1 h at room temperature in darkness. Cells are then washed 2 times in wash/permeabilization solution and resuspended in flow buffer (HBSS with Ca2+ and Mg2++ 0.1% BSA+20 mM HEPES pH 7.4). Samples are measured via flow cytometry (CytoFLEX Flow Cytometer) and analyzed in FCS Express software. After gating of single cells based on the FCS/SSC, mean fluorescence intensity (MFI) of the respective staining is exported for further analysis. Typically fold change of activation is calculated according to formula: MFI/MFI_{NC}, where MFI stands for mean fluorescent intensity of sample treated by ligand and MFI_{NC} is mean fluorescent intensity of sample without stimulation by the ligand.

### Example 10. Ligand Binding Inhibition Assay

Rationale: This assay measures the efficacy and potency of compounds to inhibit binding of fluorescently labelled CXCL12 on cells expressing WT CXCR4 receptor or mutant CXCR4 receptor.

Procedure: Jurkat cells expressing CXCR4 are washed once with assay buffer (HBSS+20 mM HEPES buffer+0.2% BSA, pH 7.4) and then incubated for 15 minutes at room temperature with test compounds diluted in assay buffer at dose-dependent concentrations. Subsequently, CXCL12-AF-647 (26 ng/ml) is added to the compound-preincubated cells. The cells are incubated for 30 min at room temperature. Thereafter, the cells are washed twice in assay buffer, fixed with 1% paraformaldehyde in PBS, and analyzed by flow cytometry (Cytoflex). Mean fluorescence intensity of CXCL12-AF647 is determined (FCS express software). The percentage of inhibition is calculated according to the formula: [1−((MFI−MFI_{NC})/(MFI_{PC}−MFI_{NC}))]*100 where MFI is the mean fluorescence intensity of cells in the presence of an inhibitor, MFI_{NC} is mean fluorescence intensity of cells in the absence of the ligand and MFI_{PC} is mean fluorescence intensity of cells in the presence of the ligand alone. Reference: pubmed.ncbi.nlm.nih.gov/29578516/.

### Example 11. Differentiation Assays

Rationale: Granulocytic differentiation defect is often observed in severe congenital neutropenia patients carrying ELANE mutations. This assay measures the efficacy of tested compounds (e.g. CXCR4 antagonists) on the granulocytic differentiation.

Procedure: Human CD34⁺ hematopoietic stem and progenitor cells (HSPCs) are isolated from bone marrow mononuclear cells using Ficoll-Paque Plus (GE Healthcare) and a human CD34 microbead kit (Miltenyi Biotec). CD34⁺ HSPCs are then expanded in StemSpan serum-free expansion medium (STEMCELL Technologies) supplied with human cytokines SCF, TPO, FLT3L, IL-6 (100 ng/ml each, all from PeproTech), UM171 (35 nM), and SR1 (0.75 mM, both from STEMCELL Technologies) for 48 h. Afterwards, CD34⁺ HSPCs are incubated with tested compounds (e.g. CXCR4 antagonists) in complete medium (RPMI 1640 GlutaMAX supplemented with 10% FBS, 1% penicillin/streptomycin, 5 ng/ml SCF, 5 ng/ml IL-3, 5 ng/ml GM-CSF and 10 ng/ml G-CSF). Medium is exchanged every second day. On day 14, the frequencies of human promyelocytes (CD45⁺CD33^{hi}CD15⁺) and mature neutrophil (CD45⁺CD33^{lo} CD15⁺CD66b⁺) are analyzed by flow cytometry (CytoFLEX Flow Cytometer) after staining of cells with respective antibodies against CD45, CD15, CD33 and CD66b (all from Biolegend). Reference: pubmed.ncbi.nlm.nih.gov/32822592/ and pubmed.ncbi.nlm.nih.gov/31248972/.

### Example 12. Phagocytosis Assay

Rationale: Phagocytosis by polymorphonuclear neutrophils and monocytes play an essential role in defending against bacterial or fungal infections. This assay measures the efficacy of tested compounds (e.g. CXCR4 antagonists) on phagocytotic uptake of polymorphonuclear neutrophils.

Procedure: The assessment of phagocytosis is analyzed using the Phagotest Kit (BDBioscience) containing fluorescein-labeled opsonized Escherichia coli (E. coli—FITC). Blood samples (90 μL) are incubated with tested compounds (e.g. CXCR4 antagonists) for 30 minutes at 37° C. Afterwards, cells are mixed with 20 μl FITC-labeled E. Coli and incubated in a chamber thermostat at 37° C. for 15 min. Simultaneously, the control samples are put into an ice in order to stop phagocytosis. Afterwards, 100 μL of brilliant blue (Quenching solution, Reagent C) is added to suppress the fluorescence of bacteria connected to the leukocyte surface. After two washing steps (with 2 mL of washing solution, solution A), erythrocytes are lysed using lysis buffer (Reagent D) for 20 min at room temperature. At the end, 200 μL DNA staining solution (reagent E) is added to stain leukocytes and bacterial DNA. The mean FITC fluorescence intensity per neutrophil (phagocytotic activity) is measured using flow cytometry (CytoFLEX Flow Cytometer) and analyzed by using flow cytometry software (FCS Express).

### Example 13. Antimicrobial Activity Assay

Rationale: This Assay Measures the Efficacy of Tested Compounds (e.g. CXCR4 Antagonists) on Antimicrobial Activity of Polymorphonuclear Neutrophils.

Procedure: Polymorphonuclear neutrophils are pre-incubated with tested compounds for 20 minutes at 37° C. Afterwards, cells are co-incubated with bacteria (e.g. S. aureus (ATCC 25923) and E. coli (ATCC 11129)) at a multiplicity of infection (MOI) of 2 in a final volume of 500 μl in 48-well non-treated cell culture plates at 37° C. for 30 minutes. Cells are lysed by addition of 50 μl of 0.25% Triton X-100 (Sigma) in DPBS (Gibco) and serial dilutions are plated on Todd-Hewitt agar plates for viable count. Total colonies are counted after incubation for 24 h at 37° C. Results are expressed as surviving bacteria compared to bacterial growth under the same conditions in the absence of cells. References: www.ncbi.nlm.nih.gov/pmc/articles/PMC5316427/ and ncbi.nlm.nih.gov/pmc/articles/PMC130096/.

### Example 14. ROS (Reactive Oxygen Species) Formation Assay

Rationale: ROS production by the phagocytotic cells is associated with pathogen killing. This assay measures the efficacy of tested compounds (e.g. CXCR4 antagonists) on ROS formation of polymorphonuclear neutrophils.

Procedure: The assessment of ROS formation is analyzed using the ROS-Glo™ H₂O₂ kit (Promega). Polymorphonuclear neutrophils are seed in 96 well plate in RPMI (Gibco) medium supplemented with 1% FBS (Sigma) at density of 5×10⁴ cells/well. Cells are incubated with tested compounds for 30 minutes at 37° C. Afterwards, cells are stimulated with 1 μM PMA (Sigma) to stimulate ROS production. After 15 minutes incubation at 37° C., 20 μl of H₂O₂ substrate is added and cells are further incubated for 15 minutes at 37° C. At the end, cells are incubated with 100 μl of ROS-Glo Detection solution for 20 minutes at room temperature. The luminescence signal, which is relative to a ROS formation, is measured by Synergy HT plate reader (BioTek).

### Example 15. ADCP, ADCC and Degranulation Assay

(ADCC): “Neutrophil-mediated antibody-dependent killing of herpes-simplex-virus-infected cells,” Siebens et. al. (1979) Blood. 54:88-94.

(ADCC): “Comparative Efficiency of HIV-1-Infected T Cell Killing by NK Cells, Monocytes and Neutrophils,” Smalls-Mantey et al. (2013) PLoS One 8:e74858.

(ADCP): “Antibody-Dependent Cellular Phagocytosis of HIV-1-Infected Cells Is Efficiently Triggered by IgA Targeting HIV-1 Envelope Subunit gp41,” Duchemin et al. (2020) Frontiers In Immunology 11:1141.

“A versatile high-throughput assay to characterize antibody-mediated Neutrophil phagocytosis,” Karsten et al. (2019) J. Immunolog Methods 471:46-56.

### Example 16. A Luminescence-Based β-Arrestin Recruitment Assay for Unmodified Receptors

See Pedersen et al. (2021) J.Biol.Chem. 296:100503. G protein-coupled receptors (GPCRs) signal through activation of G proteins and subsequent modulation of downstream effectors. More recently, signaling mediated by β-arrestin has also been implicated in important physiological functions. This has led to great interest in the identification of biased ligands that favor either G protein or β-arrestin-signaling pathways. However, nearly all screening techniques for measuring β-arrestin recruitment have required C-terminal receptor modifications that can in principle alter protein interactions and thus signaling. As described in the reference, a luminescence-based assay to measure β-arrestin recruitment to the membrane or early endosomes by unmodified receptors may be used. This strategy uses NanoLuc, an engineered luciferase from Oplophorus gracilirostris (deep-sea shrimp) that is smaller and brighter than other well-established luciferases. Recently, several publications have explored functional NanoLuc split sites for use in complementation assays. The reference has identified a unique split site within NanoLuc and fused the corresponding N-terminal fragment to either a plasma membrane or early endosome tether and the C-terminal fragment to β-arrestins, which form the basis for the MeNArC and EeNArC assays, respectively. Upon receptor activation, β-arrestin is recruited to the membrane and subsequently internalized in an agonist concentration-dependent manner. This recruitment promotes complementation of the two NanoLuc fragments, thereby reconstituting functional NanoLuc, allowing for quantification of β-arrestin recruitment with a single luminescence signal. This assay avoids potential artifacts related to C-terminal receptor modification and has promise as a new generic assay for measuring β-arrestin recruitment to diverse GPCR types in heterologous or native cells.

### Example 17. Antagonist Activity at CXCR4 (Unknown Origin) Assessed as Inhibition of SDF-1-Induced Beta-Arrestin Recruitment Incubated for 30 Mins Prior to SDF-1 Challenge Measured after 90 Mins by Chemiluminescence Assay

See Discovery of tetrahydroisoquinoline-based CXCR4 antagonists Truax et al. (2013) ACS Med Chem Lett. 4:1025-30. Compounds with activity<=50 uM or explicitly reported as active by ChEMBL are flagged as active in this PubChem assay presentation.

### Example 18. Sepsis Models

LPS-induced sepsis (endotoxemia) is a clean, endotoxemia model but not all hallmarks of bacteremia/sepsis (e.g. bacterial replication, invasion, etc.). G-CSF demonstrated efficacy in such a model (e.g. Gorgen et al., J Immunol, 1992-250 ug/kg G-CSF vs. 5mpk LPS via TNF suppression). CAVE: mice are not very sensitive to LPS-GalN-induced sensitization recommended

Monomicrobial sepsis is a direct induction of sepsis by i.v. or i.p. infection with e.g. E. coli (sensitivity can also be improved w/GalN) or S.aureus—considerably high inoculum doses (10⁹ CFU) needed as mice are not naturally susceptible. Secondary bacteremia/sepsis after i.n. infection (pneumonia->sepsis; approx. 10-100× lower inoculum needed)

Polymicrobial sepsis: CASP (=colon ascendens stent peritonitis)—less often used, technically demanding, low throughput. CLP (=cecal ligation and puncture)—standard model, technically demanding and some variability depending on source of mice and how CLP is performed; medium throughput. CSI (=cecal slurry injection)—standard model; easier to standardize than CLP; slurry can be harvested at bigger batch, frozen and used for repeat studies; however, less severe and more “artificial” than real peritoneal infection vial CLP; high throughput.

### Example 19: Analysis of Mavorixafor Effects on WBCs Across Multiple Disease States

Mavorixafor is an orally available investigational, small-molecule, selective antagonist of the CXCR4 receptor with potential to restore physiological trafficking and maturation of white blood cells (WBCs). Mavorixafor was previously shown to increase totals and subsets of WBCs in healthy volunteers and in a phase 2 clinical trial in adults with WHIM (Warts, Hypogammaglobulinemia, Infections, Myelokathexis) syndrome (Stone N, et al. Antimicrob Agents Chemother. 2007; 51(7):2351-2358; Dale D, et al. Blood. 2020; 136(26):2994-3003). We have now found that the daily oral administration of mavorixafor has notable effects on peripheral WBC counts and subsets in patients with clear cell renal cell carcinoma (ccRCC), WHIM syndrome, and Waldenstrom's macroglobulinemia (WM). Percentage changes in total peripheral WBC count, absolute neutrophil count (ANC), absolute lymphocyte count (ALC), and absolute monocyte count (AMC) from pretreatment levels were evaluated in the following settings: a phase 1/2 trial evaluating mavorixafor (200 mg twice daily or 400 mg once daily [QD]) in combination with axitinib (5 mg twice daily) in patients with advanced ccRCC who received≥1 prior therapy; a phase 1b trial evaluating mavorixafor (400 mg QD) in combination with nivolumab (240 mg QD) in patients with metastatic ccRCC unresponsive to prior nivolumab monotherapy; a long-term extension of the aforementioned phase 2 trial evaluating mavorixafor 300 or 400 mg QD in patients with WHIM syndrome with pathogenic CXCR4 gain-of-function mutation and ANC≤400/μL and/or ALC≤650/μL; and an ongoing phase 1b trial evaluating mavorixafor (200 mg QD for 4 weeks, increased to 400 mg and 600 mg QD thereafter) in combination with ibrutinib (420 mg QD) in patients with WM with MYD88 and CXCR4 mutations.

In the study evaluating combination mavorixafor (400 mg QD) and axitinib in ccRCC, total WBC count, ANC, ALC, and AMC increased to 153%, 158%, 143%, and 182% of baseline after 4 weeks (n=49), and with increases sustained at 159%, 171%, 139% and 166% of baseline after 6 months' treatment (n=20). In the study evaluating mavorixafor in combination with nivolumab in ccRCC, total WBC count, ANC, ALC, and AMC increased to 146%, 143%, 141%, and 179% of baseline after 4 weeks (n=9), and with increases sustained at 147%, 136%, 152%, and 191% of baseline after 6 months (n=2). In an interim analysis of the phase 1b trial in WM, compared to screening values, total WBC count, ANC, ALC, and AMC increased to 192%, 170%, 219%, and 186% of baseline after 4 weeks (n=8), and with increases sustained at 163%, 192%, 106%, 172% of baseline after 6 months' (n=5) treatment. In the WHIM syndrome phase 2 extension, total WBC count, ANC, ALC, and AMC increased to 339%, 652%, 239%, and 486% of baseline after 6 months' (n=5) treatment, with annualized infection rate decreasing from 5.6 (SD±3.13) events at baseline to 2.2 (SD±0.93) events after 40 months. Mavorixafor was generally well tolerated, with manageable safety profile across all indications either alone or in combination with other drugs.

Mavorixafor alone or in combination with other therapies is the first oral treatment to either acutely or chronically increase total peripheral WBCs 1.5- to 3-fold and WBC subsets across all disease populations examined, in both the presence (WHIM syndrome and WM) and absence (ccRCC and healthy volunteers) of CXCR4 gain-of-function mutation. Increases in WBC subsets occurred rapidly and were sustained during chronic treatment, with a larger treatment effect in patients with pre-existing cytopenia (WHIM syndrome) compared to patients without cytopenia at baseline (ccRCC and WM). Co-occurring reduction in infection burden was observed in the phase 2 trial in WHIM syndrome. Assessment of the beneficial effects of mavorixafor on total and WBC subsets is ongoing in a phase 3 trial of WHIM syndrome and a phase 1 trial of severe chronic neutropenia (SCN) that will assess the potential to correct cytopenias by elevating total WBC counts.

### Example 20: Chemotaxis Assay: PBMCs from a Patient with CVID Due to NFKB1 Mutation

Human peripheral blood mononuclear cells (PBMCs) are immune cells with a single, round nucleus that originate in bone marrow and are secreted into peripheral circulation. PBMCs consist of lymphocytes (T cells, B cells, NK cells) and monocytes). These cells are critical components of the immune system and are involved in both humoral and cell-mediated immunity.

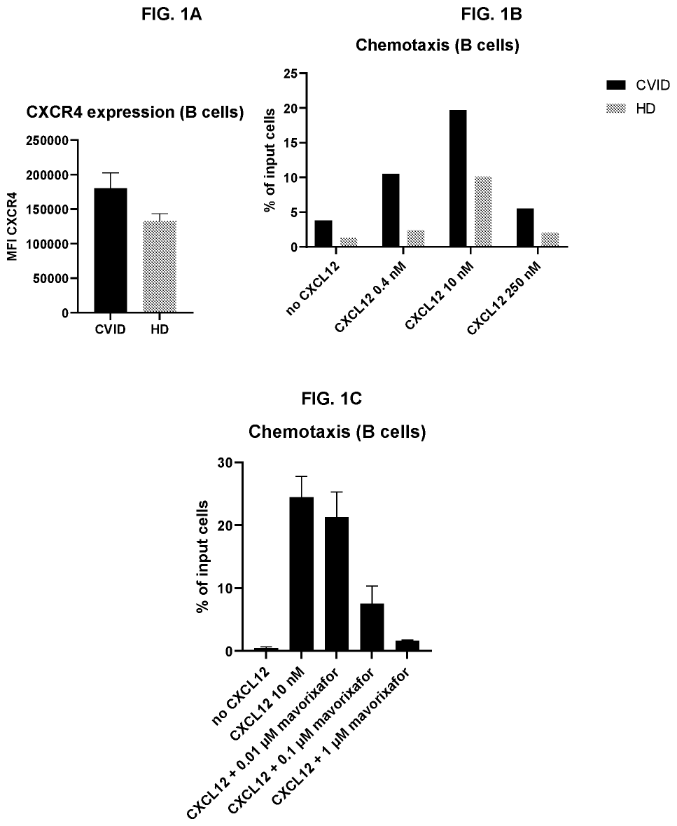

In B cells isolated from a CVID patient having an NFKB1 mutation (c.980dup p.Ala328Serfs*12) NM_003998.4(NFKB1) [“CVID B cells” ], CXCR4 expression was found to be elevated compared to B cells isolated from a healthy donor [“healthy B cells” ](180,000 MFI compared to 140,000 MFI) (FIG. 1A). CVID B cells exhibited significantly greater chemotaxis toward CXCL12 than healthy B cells, with peak chemotaxis observed at a concentration of 10 nM CXCL12 (FIG. 1B). Administration of mavorixafor to CVID B cells cultured with 10 nm CXCL12 exhibited a dose-dependent normalization effect on chemotaxis, effectively neutralizing the chemotaxis at 0.1 uM concentration mavorixafor (FIG. 1C).

### Methods and Materials: Chemotaxis in CVID B Cells and Effects of Mavorixafor on Chemotaxis.

PBMCs from patients or healthy donors were resuspended in chemotaxis buffer (RPMI 1640 media containing 20 mM HEPES, L-glutamine, and 0.5% BSA) at 2.0×10⁶ cells/mL. Cells were pretreated with the indicated concentrations of mavorixafor for 30 minutes at 37° C. with 5% CO₂. Chemotaxis was assayed immediately after treatment with drug by placing 1.0×10⁵ cells in 100 μL in the upper chamber of a Transwell 24-well plate separated by a 6.5 mm insert with 3 μm pores (Corning Life Sciences, Corning, NY, USA) from the lower chamber containing 600 μL of buffer with the indicated concentration of CXCL12. After incubation for 2.5 hours at 37° C. in a 5% CO₂ incubator, the cells that migrated into the lower chamber were collected by centrifugation and resuspended in the assay buffer. Cells were blocked using Human TruStain FcX (BioLegend, Inc) for 10 minutes at RT, and subsequently B lymphocyte subtyping was performed (Alexa Fluor® 488 Mouse anti-human CD19 IgG1, κ, HIB19, BioLegend, Cat #302219). A fixed number of flow cytometry counting beads (Precision Count Beads; BioLegend) were added to each sample.

Both migrated cells and counting beads were counted by flow cytometry (CytoFLEX). Data were analyzed using FCS Express software, and the total number of migrated cells was calculated, according to the counted and total number of beads present in the sample. Results are presented as percentage of migrated B cells out of the total number of B cells placed in the chemotaxis chamber.

### Example 21: Highly Efficient Genome Editing of Human Hematopoietic Stem/Progenitor Cells (HSPCs) to Generate Models for Assay of Effects of CXCR4 Inhibitors

CRISPR is a well-established method for gene-editing mammalian cells in order to introduce specific mutations and/or phenotypes into the cells. Methods described in Gundry et al. (2016) Cell Reports 17:1453-1461 can be adapted for mechanism-based analyses of the effects of CXCR4 inhibition on cells carrying mutations leading to primary immune deficiencies. Transfecting HSPCs isolated from Cas9-expressing mice with sgRNA is an efficient method to edit the genome of HSPCs, since only the small guide RNA molecules would need to be introduced. The resulting cells can be useful for altering HSPC function and subsequently testing the effects of both positive and negative regulators of HSPC function. Using CRISPR-based models, HSPCs can be produced with genotypic mutations that cause the primary immune deficiencies studied in the present invention (“PID cells”). These mutated PID cells can be used in assays to assess the effects of CXCR4 inhibitors on such PID disease models. For example, Rao et al. (2021) Cell Stem Cell 28:833-845 used a CRISPR-based model to knock out the function of the ELANE gene and generate cells carrying mutations leading to severe congenital neutropenia (SCN). These cells can be used for mechanism-based analysis of the effects of CXCR4 inhibitors on the SCN model by administration of mavorixafor or another CXCR4 inhibitor. Using analogous methods, CRISPR-based methods can be used to create PID cells for use in models of other primary immune deficiencies described in the present invention and can be used to assess the effects of CXCR4 inhibitors in such PID disease models, such as those described in the present invention.

### Example 22: Correction of B and T Cell Lymphopenia in WHIM Syndrome Caused by Dysregulated Stem Cell Niches and Altered Lymphocyte Recirculation

Gain-of-function (GOF) mutations in CXCR4 cause WHIM (warts, hypogammaglobulinemia, infections, and myelokathexis) syndrome, characterized by infections, leukocyte retention in bone marrow (BM), and blood leukopenias. B lymphopenia is evident at early progenitor stages, yet the CXCR4 GOF mutations that cause B (and T) lymphopenia remain obscure. Using a CXCR4 R334X GOF mouse model of WHIM syndrome, Zehentmeier and coworkers have shown that lymphopoiesis is reduced because of a dysregulated mesenchymal stem cell (MSC) transcriptome characterized by a switch from an adipogenic to an osteolineage-prone program with limited lymphopoietic activity. Zehentmeier et al., Sci. Immunol. 7, eabo3170 (2022), 23-9-2022. They identified lymphotoxin beta receptor (LTβR) as a critical pathway promoting interleukin-7 (IL-7) down-regulation in MSCs. Blocking LTβR or CXCR4 signaling restored IL-7 production and B cell development in WHIM mice. LTβR blocking also increased production of IL-7 and B cell activating factor (BAFF) in secondary lymphoid organs (SLOs), increasing B and T cell numbers in the periphery. These studies revealed that LTβR signaling in BM MSCs and SLO stromal cells limits the lymphocyte compartment size. In these studies, the following CXCR4 inhibitor was used:

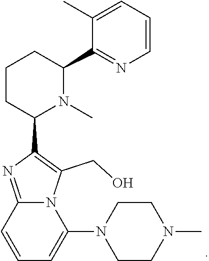 [SMILES: Cc1cccnc1[C@@H]1CCC[C@H](c2nc3cccc(N4CCN(C)CC4)n3c2CO)N1C]

Study design. The purpose of this study was to understand mechanistically how GOF mutations in CXCR4 cause mild lymphopenia. To do this, the authors generated a mouse model of the CXCR4 R334X mutation causing WHIM syndrome (WHIM mice) and confirmed systemic reduction in B and T cell numbers in WHIM mice. They analyzed stromal cell subsets in the BM and SLOs by bulk and scRNA-seq and identified 117 down-regulation in bone marrow mesenchymal stem cells (BM MSCs) as a mechanism driving lymphopenia. They further identified LTβR as the mechanism causing 117 down-regulation in vivo. These conclusions were reached by using LTβR- and CXCR4-specific antagonists in vivo using groups of three to five mice per experimental condition and further validated using genetic tools that allow the deletion of Ltbr in BM MSCs. Mice of both sexes were included in this study but examined in independent experiments. All experiments described in this study were replicated independently at least twice with the exception of RNA-seq experiments that were performed once.

Patient samples. Plasma samples from 6 patients with WHIM, 21 patients with partial RAG deficiency, and 20 healthy controls were obtained from J. Walter's biobank at the University of South Florida. All subjects were recruited according to protocols approved by the local Institutional Review Boards (IRBs) as follows: USF-Pro00035468 [principal investigator (PI): J.E.W.], USF-Pro00025693 (PI: J.E.W.), JHMI-IRB00175372 (PI: J.E.W.), JHMI-IRB00097062 (PI: J.E.W.), and JHMI-IRB00097938 (PI: J.E.W.).

Mice. Cxcr4WHIM/+ mice were generated at the Yale Genome Editing Center using CRISPR-Cas technology. RNA guides were designed to introduce c.1021C>T and c.1023G>A mutations in exon 2 of murine Cxcr4, leading to the exchange of arginine-341 with a STOP codon (R341X; see FIG. S1A). Mice used for experiments were backcrossed to C57BL/6J mice 5 to 10 generations (Jackson Laboratory, strain code 000664). Adult C57BL/6NCr (strain code 556, CD45.2+) and B6-Ly5.1/Cr (strain code 564, CD45.1+) mice for experiments were purchased from Charles River Laboratories. LeprCre/+ mice were purchased from the Jackson Laboratory. Ltbrfl/fl and Ltb−/− mice (65), Il7GFP/+ mice (66), and Cxcll2DsRed/+ mice were from an internal colony. Male and female adult mice (6 to 21 weeks old) were used at 50% ratios. All mice were maintained under specific pathogen-free conditions at the Yale Animal Resources Center and were used according to the protocol approved by the Yale University Institutional Animal Care and Use Committee.

LTβR signaling inhibition. LTβR-Ig or HEL-Ig (100 to 150 μg) was injected intravenously once and analyzed 1 week later or once a week for 3 weeks.

CXCR4 signaling inhibition. Mice were gavaged daily with the CXCR4 antagonist of the structure shown above in 50 mM citrate buffer (pH 4.0) for 1 or 3 weeks at 100 mg/kg or with vehicle (for control groups). The compound is orally bioavailable.

Lymphocyte homing assay. B cells or T cells were isolated from Cxcr4WHIM/+ and Cxcr4+/+ spleens and peripheral lymph nodes by magnetic separation using the EasySep Mouse B cell Isolation Kit or EasySep Mouse T cell Isolation Kit (STEMCELL Technologies). T and B cells were fluorescently labeled with CFSE or CMTMR, and a total of 1×107 mixed B cells or 1.5×107 mixed T cells at a ratio of 1:1 were adoptively transferred intravenously into recipient mice. Mice were analyzed 16 to 20 hours after adoptive transfers.

Flow cytometry. Single-cell suspensions of the spleens, peripheral lymph nodes (axillary, brachial, and inguinal), and thymi were prepared as previously described. BM cells were flushed from long bones using the same medium. BM stromal cells were isolated as previously described. Stromal cells from lymph nodes were isolated by collagenase IV and deoxyribonuclease (DNase) I digestion. Lymph nodes were cleaned from surrounding fat tissue and cut into ˜1-mm-thick slices using a razor blade; placed into Hanks' balanced salt solution (HBSS) supplemented with 2% heat-inactivated fetal bovine serum (FBS), 1% penicillin/streptomycin, 1% L-glutamine, and 1% Hepes; and incubated for 20 min at 37° C. Then, collagenase IV (1800 U/ml; Worthington Biochemical Corporation) and Dnase I (80 g/ml; Sigma-Aldrich) in the same media were added at a ratio of 1:1 to reach final concentrations of 900 U/ml for collagenase IV and 40 g/ml for DNase I, and lymph node slices were digested for 20 min at 37° C. Next, the tissue was gently disrupted by pipetting and incubated for another 10 min at 37° C. Cells were carefully re-suspended, and suspensions were centrifuged at 1200 rpm for 10 min and resuspended in HBSS supplemented as above. Cell concentrations were determined with a Coulter counter (Beckman Coulter). Cells were stained with antibody cocktails diluted in FACS buffer on ice at a concentration of 25 μl/1×10⁶ cells. The following antibodies were used (BioLegend unless indicated otherwise): anti-mouse CD3e (1452C11), CD4 (GK1.5), CD8 (535.8), CD11b (M1/70), CD11c (N418), CD19 (6D5), CD23 (B3B4), CD25 (PC61), CD44 (IM7), CD45.1 (A20), CD45.2 (104), B220 (RA3-6B2), CD71 (RI7217), CD93 (AA4.1), CD115 (AFS98), CD117 (c-Kit, 2B8), IgM (II/41, Thermo Fisher Scientific), IgD (11-26c.2a), Gr1 (RB6-8C5), and Ter119 (Ter119). Hematopoietic cell populations were identified as follows: pro-B, CD19+ CD93+ IgM− c-Kit+ or CD19+ CD93+ IgM− FSChigh; pre-B, CD19+ CD93+ IgM− c-Kit− or CD19+ CD93+ IgM− FSClow; immature B, CD19+ CD93+ IgM+; mature B, CD19+ (or B220+) CD93− IgM+ IgD+ or CD19+ (or B220+) CD93− CD23+; T1 B cells, CD19+ (or B220+) CD93+ IgM+ CD23− (or IgD−); T2 B cells, CD19+ (or B220+) CD93+ CD23+ (or IgD+); CD4+ T cells, CD3e+ CD4+; CD8+ T cells, CD3e+ CD8+; thymocytes: double negative (DN) 1, CD4− CD8− CD44+ CD25−; DN2, CD4− CD8− CD44+ CD25+; DN3, CD4− CD8− CD44− CD25+; and DN4, CD4− CD8− CD44− CD25−; double positive (DP), CD4+ CD8+; CD4 single positive (SP4), CD4+; CD8 single positive (SP8), CD8+; neutrophils, CD11b+ CD115− Grlhigh; and monocytes, CD115+ Grlint. The following antibodies were used to identify nonhematopoietic cell populations: anti-mouse GP38 (8.1.1); CD31 (390); CD144 (BV13); LEPR (goat polyclonal, R&D Systems); LTβR (5G11); MSC, CD45− Ter119− CD31− CD144− LEPR+; and lymph node GP38+ SC, CD45− Ter119− CD31− GP38+. For live/dead cell discrimination, cells were stained with 4′,6-diamidino-2-phenylindole (DAPI).

For detection of apoptotic cells, cells were first stained for surface proteins; then, cells were washed twice with FACS buffer and stained for 15 min at room temperature (RT) with fluorescently labeled annexin V in annexin V binding buffer (BioLegend). Samples were recorded with LSR II cytometers (BD) and FACSDiva software (BD) and analyzed with FlowJo software (BD).

Flow cytometric detection of BAFF. Cells were stained with anti-BAFF [clone 121808, R&D Systems, fluorescein isothiocyanate (FITC)-coupled]. For intracellular staining, cells were fixed with Cytofix/Cytoperm solution (BD) for 20 min on ice and washed twice with Perm/Wash buffer (BD). Then, cells were stained for 30 min at RT with anti-BAFF and then for 20 min at RT with a goat anti-rat IgG-BV421 antibody (BioLegend).

Immunofluorescence microscopy. Tissue fixation and cryosectioning were performed as described. Sections were rehydrated in phosphate-buffered saline (PBS) for 20 min, blocked with 5% FBS in PBS/0.1% Tween 20 for 20 min, and stained with primary antibodies for 1 hour. Sections were washed five times with PBS/0.1% Tween 20, and secondary antibodies were applied for 1 hour. Slides were mounted with Fluorescence Mounting Medium (Dako). Images were acquired on a Zeiss Z1 observer fluorescent microscope equipped with Colibri light-emitting diode light sources or with Leica SP8 confocal microscope. The following primary antibodies were used: anti-MAdCAM1 (MECA-367, BioLegend), rabbit anti-red fluorescent protein (polyclonal, Rockland), CD3-biotin (145-2C11), and CD35-biotin (8C12, BD). The following secondary reagents were used: donkey anti-rat-IgG (H+L) Alexa Fluor 488 (Jackson ImmunoResearch), donkey anti-rabbit-IgG (H+L) Alexa Fluor 555 (Thermo Fisher Scientific), streptavidin-Alexa Fluor 488, and streptavidin-Alexa Fluor 555 (Thermo Fisher Scientific). The following conjugated antibodies were used: IgD-Alexa Fluor 647 (11-26c.2a, BioLegend) and CD21/35-FITC (7E9). Nuclei were labeled with DAPI. Images were analyzed with ImageJ (68) and Zen (Zeiss).

Preparation of MSCs for RNA-seq. Long bones were flushed with HBSS supplemented with 2% heat-inactivated FBS, 1% penicillin/streptomycin, 1% L-glutamine, 1% Hepes, and collagenase IV (200 U/ml). The flush out was digested for 30 min at 37° C.; cell clumps were dissociated by gentle pipetting, and cells were filtered through a 100-μm nylon mesh and washed with HBSS supplemented with 2% FBS, 1% penicillin/streptomycin, 1% L-glutamine, and 1% Hepes. Hematopoietic cells were stained with biotin-conjugated CD45 and Ter119 antibodies and magnetically depleted using Dynabeads Biotin Binder (Thermo Fisher Scientific). The remaining cells were stained with antibodies against CD31, CD144, and LEPR; fluorochrome-conjugated streptavidin; and DAPI. Sorting was performed using BD FACSAria II, and MSCs were identified as CD45− Ter119− CD31− CD144− LEPR+ cells. Cells were sorted into Dulbecco's modified Eagle's medium (DMEM)/10% FBS and resorted with the same strategy directly into 350 μl of RLT plus buffer (QIAGEN).

RNA-seq of BM stromal cells. RNA-seq was performed using the Illumina HiSeq 2500 system, with paired-end 2×76-base pair (bp) read length. Using the TopHat v2.1.0 software, the sequencing reads were aligned onto the Mus musculus GRCm38/mm10 reference genome. Using HTSeq v0.8.0 software, the mapped reads were converted into the count matrix with default parameters, followed by the variance stabilizing transformation offered by DESeq2. DEGs were identified using the same software on the basis of a negative binomial generalized linear models and visualized in hierarchically clustered heatmaps using the pheatmap package in R.

scRNA-seq of BM stromal cells. scRNA-seq was performed by the Yale Center for Genome Analysis. The libraries were prepared using the Chromium Single Cell 3′ Reagent Kits v3.0 according to the protocol and run on an Illumina NovaSeq system with 100-bp paired-end reads to a coverage of ˜40,000 to 44,000 mean reads per cell and ˜80% saturation. The sequencing reads were aligned onto the M. musculus GRCm38 (mm10) reference genome.

Preparation of SLO stromal cells for scRNA-seq. Peripheral lymph node stromal cell suspensions were prepared from two mice per genotype and pooled. Cells were stained with biotinylated antibodies against CD45 and Ter119, and hematopoietic cells were depleted using Dynabeads Biotin Binder (Thermo Fisher Scientific). The leftover cells were stained with streptavidin-BV711 (BioLegend); DAPI; and antibodies against CD31, CD71, and GP38. Lymph node stromal cells were sorted as CD45− Ter119− CD71− CD31− cells into 350 μl of DMEM with 20% FBS. GP38 was used to control for the presence of stromal cells in the suspension. Splenic stromal cell suspensions were prepared from three mice per genotype and pooled. The leftover cells were stained for CD45, Ter119, LIN (CD3e, B220, and CD19), CD31, GP38, and platelet-derived growth factor receptor α (PDGFRα). Splenic stromal cells were sorted as CD45− Ter119− LIN− CD71− CD31− into 350 μl of DMEM with 20% FBS. GP38 and PDGFRα were used to control for the presence of stromal cells in the suspension. scRNA-seq was performed as described for BM stromal cells to a coverage of ˜65,000 mean reads per cell and >90% saturation for lymph node stromal cells and to a coverage of ˜30,000 to 57,000 mean reads per cell and >80% saturation for splenic stromal cells. The sequencing reads were aligned onto the M. musculus GRCm38 (mm10) reference genome.

BM Stromal Cell scRNA-Seq

Preprocessing of scRNA-seq data. The Gene-Barcode matrix generated from 10× Genomic Cell Ranger was preprocessed using the Seurat (version 3.2.3) R package. Briefly, cells that had fewer than 200 or more than 6000 detected features and more than 25% of mitochondrial gene content were excluded to remove low-quality cells. Subsequently, contaminating hematopoietic cells were filtered out from the down-stream analysis. After quality control, SCTransform was used to normalize the count data and stabilize variance.

Dimension reduction and clustering. Highly variable genes were identified and used for dimensionality reduction of the entire 12,598 cell dataset with principal components analysis (PCA). IKAP [identifying K major cell population groups in scRNA-seq analysis] was used to estimate the number of optimal principal components (PCs) and clusters. Cell clustering was performed using the FindClusters function of Seurat and visualized in a two-dimensional space using the UMAP visualization method. MSC clusters were further subjected to Markov affinity-based graph imputation of cells to restore the structure of the data by imputing plausible gene expression in each cell.

Cell type annotation. Pairwise differential expression analysis was performed for each cluster using the model-based analysis of single-cell transcriptomics (MAST) method to identify marker genes. Cluster-specific marker genes were matched to the cell types on the basis of the previously identified expression signature and the known marker expression in the publicly available databases PanglaoDB and the Human Protein Atlas. The top 10 significant positive marker genes were selected to generate feature plots and heatmap plots.

Differential abundance test. To conduct differential abundance analysis of neighborhoods, R implementation of Milo (github.com/MarioniLab/miloR) were applied. Cell neighborhoods were defined under parameters (k=12, d=20), and the proportion of randomly sampled graph vertices was set to prop=0.1. Cell neighborhoods with statistically significant differential abundances between two groups were determined, and plotDAbeeswarm was used to display abundance differences.

SLO stromal cell scRNA-seq data preprocessing and analysis. scRNA-seq data from lymph node and splenic stromal cells were analyzed using SCANPY. For this, scatter plots were first manually inspected for total gene expression count (500 to 20,000), the number of expressed genes (>100), and mitochondrial gene expression fraction (<0.2 for the lymph node data and <0.1 for the spleen data) to filter and preprocess the data. The lymph node data resulted in 12,521 cells (Cxcr4WHIM/+, 7332; Cxcr4+/+, 5189) and 17,944 genes. The spleen data resulted in 19,741 cells (Cxcr4WHIM/+: 13,660; Cxcr4+/+: 6081) and 18,362 genes. Log normalization and PCA with 50 PCs were performed, and 10 neighbors were used for UMAP visualization. Next, all contaminating cells with nonzero expression of any of the 44 hematopoietic marker genes were removed. Gene expression distributions were manually inspected in histograms, ordered dot plots, and UMAP plots. The filtering resulted in 3113 cells for the lymph node data (Cxcr4WHIM/⁺, 2131; Cxcr4^{+/+}, 982) and 3484 cells for the spleen data (Cxcr4WHIM/⁺, 1283; Cxcr4^{+/+}, 2201). For unsupervised clustering, Leiden clustering with resolutions of 0.2 and 0.1 for the lymph node and spleen data, respectively, was performed (after manual inspection of several resolution choices in UMAP plots). Eight and 12 clusters were identified for the lymph node and spleen data, respectively.

For DEG analysis, Wilcoxon rank-sum tests were performed in two ways: (i) for each cluster against all the rest and (ii) for the Cxcr4WHIM/+ group against the Cxcr4+/+ group in each cluster. Marker genes used for excluding hematopoietic contaminants were as follows: general hematopoietic markers (Ptprc, Ptpn22, Itga4, and Gpr183), B lineage (Igkc, Igha, Ighm, Ighd, Jchain, Iglv1, Iglv2, Iglv3, Iglc1, Iglc2, Iglc3, Igll1, Rag1, Rag2, Il7r, Vpreb1, Vpreb2, Vpreb3, Cd19, Cd79a, Cd79b, and Dntt), T and natural killer lineage (Ccr7, Cd3e, Cd2, Gmza, and Gmzb), myeloid lineage (Il1b, Ccr1, Cxcr2, Siglech, Ccr9, Lair1, Csf3r, Trem1, and Cd300c), and erythroid lineage (Ciita, Slc4a1, Rhd, and Alas2).

Lymph node stromal cell preparation for quantitative realtime polymerase chain reaction. Lymph node stromal cell suspension were prepared as described and sorted as CD45− Ter119− CD31− GP38+ cells directly into 350 μl of RLT plus buffer with 3.5 μl of 0-mercaptoethanol (QIAGEN) using BD FACSAria II. RNA was extracted from the lysates using the RNeasy Plus Micro Kit (QIAGEN).

RNA isolation and quantitative real-time polymerase chain Reaction. The total RNA was isolated from sorted stromal cells using the RNeasy Plus Micro Kit (QIAGEN). cDNA was synthetized from the isolated RNA using SuperScript III Reverse Transcriptase, oligo(dT)12-18 primer, deoxynucleoside triphosphates, and RNaseOUT ribonuclease inhibitors (all Thermo Fisher Scientific). Quantitative polymerase chain reaction (PCR) was performed with the SensiFAST SYBR Lo-ROX Kit (Bioline) and the CFX Touch Real-Time PCR Detection System (Bio-Rad). Hprt mRNA levels were used as control. PCR primer sequences were used as described in the reference.

Human BAFF serum enzyme-linked immunosorbent assay. BAFF plasma concentrations were measured using commercial enzyme-linked immunosorbent assay (R&D Systems, Minneapolis, MN).

Statistical analysis. Statistical analyses were performed using Prism 8 (GraphPad). Unpaired two-tailed Student's t tests were used to determine statistical significance.

We claim:

1. A method for treating a primary immunodeficiency (PID) in a subject in need thereof, comprising administering to the subject an effective amount of a CXCR4 inhibitor or a pharmaceutically acceptable salt or composition thereof.

2. The method of claim 1, wherein the CXCR4 inhibitor is selected from mavorixafor, 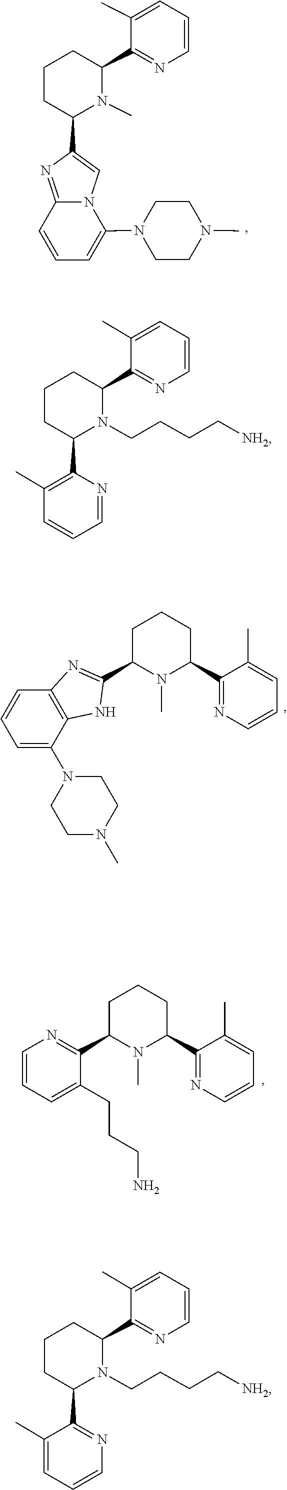 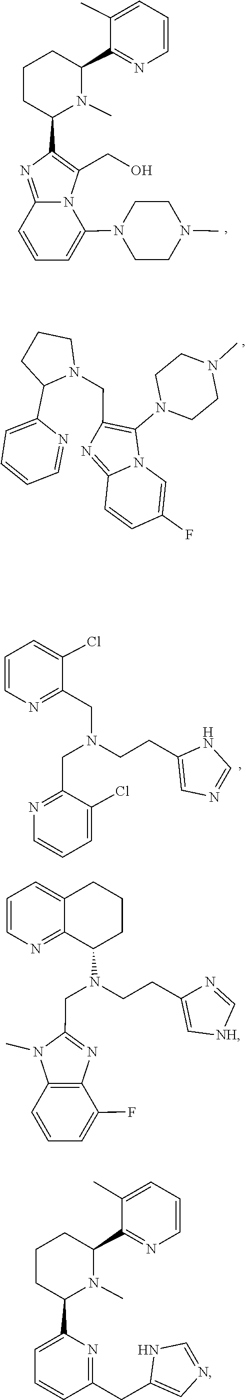 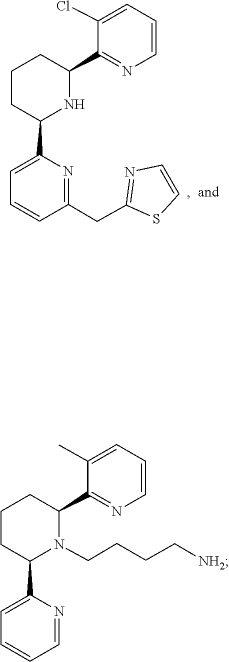 [SMILES: Clc1cccnc1[C@@H]1CCC[C@H](c2cccc(Cc3nccs3)n2)N1 ; Cc1cccnc1[C@@H]1CCC[C@H](c2ccccn2)N1CCCCN]
or a pharmaceutically acceptable salt or composition thereof.

3. The method of claim 1, wherein the CXCR4 inhibitor is mavorixafor or a pharmaceutically acceptable salt or composition thereof.

4. The method of claim 1, wherein the CXCR4 inhibitor is 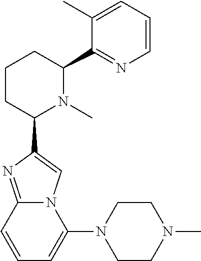 [SMILES: Cc1cccnc1[C@@H]1CCC[C@H](c2cn3c(N4CCN(C)CC4)cccc3n2)N1C]
or a pharmaceutically acceptable salt thereof.

5. The method of any of claims 1-4, wherein the PID is a common variable immune deficiency (CVID) or a disease or disorder associated with CVID.

6. The method of claim 5, wherein the CVID or disease or disorder associated with CVID is selected from recurrent infections, polyclonal lymphoproliferation, autoimmune cytopenias, granulomatous disease, severe bacterial infections, PTEN deficiency, activated p110δ syndrome, ARHGEF1 deficiency, SEC61A1 deficiency, RAC2 deficiency, SH3KBP1 deficiency, CD20 deficiency, TACI deficiency, BAFF receptor deficiency, TWEAK deficiency, IRF2BP2 deficiency, CD19 deficiency, CD81 deficiency, CD21 deficiency, TRNT1 deficiency, NFKB1 deficiency, NKFB2 deficiency, IKAROS deficiency, ATP6AP1 gene, ATP6AP1 deficiency, and MOGS deficiency.

7. The method of claim 5 or 6, wherein the patient has mutations in one or more genes selected from PI3KCD (GOF), PIK3R1, PTEN, ARHGEF1, SH3KBP1, SEC61A1, RAC2, CD20, TACI, TNFRSF13C, TWEAK, IRF2BP2, CD19, CD81, CD21, TRNT1, NFKB1, NFKB2, IKZF1, ATP6AP1, and MOGS.

8. The method of any one of claims 1-4, wherein the disease or disorder associated with primary immunodeficiency is selected from DiGeorge Syndrome, CHARGE syndrome, Chromosome 10p13-p14 deletion syndrome, Jacobsen syndrome, FOXN1 haploinsufficiency, cartilage hair hypoplasia, Schimke Syndrome, MOPD1 deficiency, MYSM1 deficiency, Facial dysmorphism, Immunodeficiency, Livedo, and Short stature (FILS) Syndrome and Mannose-Binding Lectin (MBL) deficiency.

9. The method of claim 8, wherein the patient has mutations in one or more genes selected from, TBX1, CHD7, SEMA3E, 10p13-p14DS, 11q23DEL, FOXN1, RMRP, SMARCAL1, RNU4ATAC, MYSM1, POLE, MASP2 and FCN3.

10. The method of claim 8 or 9, wherein the disease or disorder associated with primary immunodeficiency is DiGeorge Syndrome.

11. The method of any of claims 1-3, wherein the PID is a disease or disorder associated with innate immunity defects.

12. The method of claim 11, wherein the disease or disorder associated with innate immunity defects is selected from predisposition to invasive bacterial infections comprising meningitis, sepsis, osteomyelitis, and/or abscesses; IRAK4 deficiency comprising skin infections and upper respiratory tract infections; IRAK-1 deficiency comprising X-linked MECP2 deficiency-related syndrome; TIRAP, comprising staphylococcal disease during childhood; and isolated congenital asplenia, comprising bacteremia, no-spleen, hemolysis, nephritis, and inflammation.

13. The method of claim 12, wherein the disease or disorder associated with innate immunity defects is IRAK4 deficiency and wherein the patient has a mutation in IRAK4 gene; wherein the disease or disorder associated with innate immunity defects is IRAK-1 deficiency and wherein the patient has a mutation in IRAK1 gene; wherein the disease or disorder associated with innate immunity defects is TIRAP deficiency and wherein the patient has a mutation in TIRAP gene; wherein the disease or disorder associated with innate immunity defects is isolated congenital asplenia and wherein the patient has a mutation in RPSA gene; or wherein the disease or disorder associated with innate immunity defects is isolated congenital asplenia and wherein the patient has a mutation in HMOX gene.

14. The method of claim 11, wherein the disease or disorder associated with innate immunity defects is selected from predisposition to parasitic and fungal infections; mucocutaneous candidiasis, comprising chronic mucocutaneous candidiasis without ectodermal dysplasia; treating STAT1 (GOF), comprising fungal infections, bacterial infections, viral infections, HSV, autoimmunity, thyroiditis, diabetes, cytopenias, and enteropathy; IL-17F deficiency, comprising folliculitis; IL-17RA deficiency, comprising folliculitis, susceptibility to S. aureus and susceptibility to skin infections; IL-17RC deficiency; ACT1 deficiency, comprising blepharitis, folliculitis and macroglossia; CARD9 deficiency, comprising predisposition to invasive fungal diseases, predisposition to invasive candidiasis infection, and deep dermatophytosis; and trypanosomiasis.

15. The method of claim 14, wherein the disease or disorder associated with innate immunity defects is STAT1 (GOF) and wherein the patient has a mutation in STAT1 gene; the disease or disorder associated with innate immunity defects is IL-17F deficiency and wherein the patient has a mutation in IL17F gene; the disease or disorder associated with innate immunity defects is IL-17RA deficiency and wherein the patient has a mutation in IL17RA gene; the disease or disorder associated with innate immunity defects is IL-17RC deficiency and wherein the patient has a mutation in IL17RC gene; the disease or disorder associated with innate immunity defects is ACT1 deficiency and wherein the patient has a mutation in ACT1 gene; the disease or disorder associated with innate immunity defects is CARD9 deficiency and wherein the patient has a mutation in CARD9 gene; or wherein the disease or disorder associated with innate immunity defects is trypanosomiasis and wherein the patient has a mutation in APOL1 gene.

16. The method of claim 11, wherein the disease or disorder associated with innate immunity defects is selected from osteopetrosis, comprising hypocalcemia, neurologic features and severe growth retardation; hidradenitis suppurativa, comprising acne and hyperpigmentation; acute liver failure due to NBAS deficiency, comprising fever induced liver failure; acute necrotizing encephalopathy, comprising fever induced acute encephalopathy; and TRF4 haploinsufficiency, comprising Whipple's disease.

17. The method of claim 16, wherein the disease or disorder associated with innate immunity defects is osteopetrosis and wherein the patient has mutations in one or more genes selected from TNFRSF11A, PLEKHM1, and TCIRG1; wherein the disease or disorder associated with innate immunity defects is hidradenitis suppurativa and wherein the patient has mutations in one or both of PSENEN and NCSTN genes; or wherein the disease or disorder associated with innate immunity defects is acute liver failure due to NBAS deficiency and wherein the patient has a mutation in NBAS gene; wherein the disease or disorder associated with innate immunity defects is acute necrotizing encephalopathy and wherein the patient has a mutation in RANBP2 gene; or wherein the disease or disorder associated with innate immunity defects is IRF4 haploinsufficiency and wherein the patient has a mutation in IRF4 gene.

18. The method of claim 11, wherein the disease or disorder associated with innate immunity defects is selected from severe phenotypes of Mendelian susceptibility to mycobacterial disease, complete IFNGR1 deficiency, moderate phenotypes of Mendelian susceptibility to mycobacterial disease, IL-12 and IL-23 receptor b1 chain deficiency, IL-12Rb2 deficiency, IL-23R deficiency, STAT1 (LOF), partial IFNTR1, partial IFNγR2, AD IFNGR1, partial SPPL2a deficiency, partial Tyk2 deficiency, macrophage gp91 phox deficiency, IRF8 deficiency, IFG15 deficiency, RORγT deficiency, JAK1 (LOF), epidermodysplasia verruciformis (HPV), partial EVER1 deficiency, partial EVER2 deficiency, partial CIB1 deficiency, WHIM, predisposition to severe viral infection, STAT1 deficiency, STAT2 deficiency, IRF7 deficiency, IRF9 deficiency, IFNAR1 deficiency, IFNAR2 deficiency, CD16 deficiency, MDAS deficiency, RNA polymerase III deficiency, IL-18BP deficiency and herpes simplex encephalitis.

19. The method of claim 18, wherein the patient has mutations in one or more genes selected from IFNGR1, IFNGR2, IL12RB1, IL12RB2, IL23R, STAT1, IFNGR1, IFNGR2, SPPL2A, TYK2, CYBB, IRF8X, ISG15, RORC, JAK1, TMC6, TMC8, CIB1, CXCR4, STAT2, IRF7, IRF9, IFNAR1, IFNAR2, FCGR3A, IFIH1, POLR3A, POLR3C, POLR3F, IL18BP, UNC93B1, TRAF3, TICAM1, TBK1 and IRF3.

20. The method of any of claims 1-3, wherein the PID is a disease or disorder associated with functional defects of phagocytes.

21. The method of claim 20, wherein the disease or disorder associated with functional defects of phagocytes is selected from Leukocyte Adhesion Deficiency Type 1, Leukocyte Adhesion Deficiency Type 2, Leukocyte Adhesion Deficiency Type 3, pulmonary alveolar proteinosis, chronic granulomatous disease, Rac-2 deficiency and G6PD deficiency Class 1.

22. The method of claim 11, wherein the patient has mutations in one or more genes selected from, ITGB2, SLC35C1, FERMT3, CSF2RA, CSF2RB, NCF1, CYBA, NCF4, NCF2, CYBC1, RAC2 and G6PD.

23. The method of any of claims 1-3, wherein the PID is a disease or disorder associated with congenital defects of phagocyte number, function, or both in a patient.

24. The method of claim 23, wherein the disease or disorder associated with congenital defects of phagocyte number, function, or both is selected from Shwachman-Diamond Sydrome, SRP54 deficiency, glycogen storage disease type 1B, Cohen syndrome, 3-Methylglutaconic aciduria, Barth Syndrome, Clericuzio syndrome, VPS45 deficiency, JAGN1 deficiency, WDR1 deficiency, SMARCD2 deficiency, specific granule deficiency, HYOU1 deficiency, P14/LAMTOR2 deficiency, Elastase deficiency, HAX1 deficiency, GFI 1 deficiency, G-CSF receptor deficiency and neutropenia with combined immune deficiency.

25. The method of claim 24, wherein the patient has mutations in one or more genes selected from DNAJC21, EFL1, SBDS, SRP54, G6PTI, COH1, CLPB, TAZ, C160RF57, VPS45, JAGN1, WDR1, SMARCD2, CEBPE, HYOU1, LAMTOR2, ELANE, HAX1, GFI1, CSF3R, and MKL1.

26. The method of any one of claims 1-25, wherein the CXCR4 inhibitor is mavorixafor or a pharmaceutically acceptable salt thereof and the mavorixafor or pharmaceutically acceptable salt thereof is administered at a daily dose of from about 100 mg/day to about 600 mg/day; from about 200 mg/day to about 600 mg/day; from about 300 mg/day to about 500 mg/day; from about 350 mg/day to about 450 mg/day; or about 400 mg/day.

27. The method of claim 1, wherein the PID is selected from a common variable immune deficiency (CVID) or a disease or disorder associated with CVID.

28. The method of claim 27, wherein the CXCR4 inhibitor is mavorixafor or a pharmaceutically acceptable salt thereof and the mavorixafor or pharmaceutically acceptable salt thereof is administered at a daily dose of from about 100 mg/day to about 600 mg/day; from about 200 mg/day to about 600 mg/day; from about 300 mg/day to about 500 mg/day; from about 350 mg/day to about 450 mg/day; or about 400 mg/day.
